(19) United States
(12) Patent Application Publication
(10) Pub. No.: US 2007/0275193 A1
(43) Pub. Date: Nov. 29, 2007

(54) Functional Materials and Novel Methods for the Fabrication of Microfluidic Devices

(76) Inventors: Joseph M. DeSimone, Chapel Hill, NC (US); Jason P. Rolland, Durham, NC (US); Ginger Denison Rothrock, Durham, NC (US)

Correspondence Address: JENKINS, WILSON, TAYLOR & HUNT, P. A., 3100 TOWER BLVD., Suite 1200, DURHAM, NC, 27707, US

(21) Appl. No.: 10/589,222

(22) PCT Filed: Feb. 14, 2005
(86) PCT No.: PCT/US05/04421

§ 371 Date: May 11, 2007

Related U.S. Application Data
(60) Provisional application No. 60/544,905, filed on Feb. 13, 2004.

(51) Int. Cl. B32B 7/02; B05D 1/40; B05D 3/02; C08F 16/24; C12M 1/40; H05H 1/00; G01F 13/00; C08G 73/24; B67D 5/00; B05D 3/06

(52) U.S. Cl. 428/34.1; 137/3; 216/94; 216/95; 222/71; 427/240; 427/331; 427/532; 427/536; 427/559; 428/98; 526/247; 526/249; 526/250; 526/253; 526/254; 526/255; 528/25; 528/271; 528/367; 528/369; 528/401

(57) ABSTRACT

The presently disclosed subject matter provides functional perfluoropolyether (PFPE) materials for use in fabricating and utilizing microscale devices, such as a microfluidic device. The functional PFPE materials can be used to adhere layers of PFPE materials to one another or to other substrates to form a microscale device. Further, the presently disclosed subject matter provides a method for functionalizing the interior surface of a microfluidic channel and/or a microtiter well. Also the presently disclosed subject matter provides a method for fabricating a microscale structure through the use of a sacrificial layer of a degradable material.

### CROSS REFERENCE TO RELATED APPLICATIONS

This application is based on and claims priority to U.S. Provisional Patent Application Ser. No. 60/544,905, filed Feb. 13, 2004, which is incorporated herein by reference in its entirety.

### GOVERNMENT INTEREST

This invention was made with U.S. Government support from Office of Naval Research No. N000140210185 and STC program of the National Science Foundation under Agreement No. CHE-9876674. The U.S. Government has certain rights in the invention.

### TECHNICAL FIELD

The presently disclosed subject matter relates to functional materials and their use for fabricating and utilizing micro- and nano-scale devices.

### ABBREVIATIONS

AC=alternating current

Ar=Argon

° C.=degrees Celsius

cm=centimeter

8-CNVE=perfluoro(8-cyano-5-methyl-3,6-dioxa-1-octene)

CSM=cure site monomer

CTFE=chlorotrifluoroethylene

g=grams

h=hours

1-HPFP=1,2,3,3,3-pentafluoropropene

2-HPFP=1,1,3,3,3-pentafluoropropene

HFP=hexafluoropropylene

HMDS=hexamethyldisilazane

IL=imprint lithography

MCP=microcontact printing

Me=methyl

MEA=membrane electrode assembly

MEMS=micro-electro-mechanical system

MeOH=methanol

MIMIC=micro-molding in capillaries

mL=milliliters

mm=millimeters

mmol=millimoles

Mₙ=number-average molar mass

m.p.=melting point

mW=milliwatts

NCM=nano-contact molding

NIL=nanoimprint lithography

nm=nanometers

Pd=palladium

PAVE perfluoro(alkyl vinyl)ether

PDMS=poly(dimethylsiloxane)

PEM=proton exchange membrane

PFPE=perfluoropolyether

PMVE perfluoro(methyl vinyl)ether

PPVE perfluoro(propyl vinyl)ether

PSEPVE=perfluoro-2-(2-fluorosulfonylethoxy)propyl vinyl ether

PTFE=polytetrafluoroethylene

SAMIM=solvent-assisted micro-molding

SEM=scanning electron microscopy

Si=silicon

TFE=tetrafluoroethylene

μm=micrometers

UV=ultraviolet

W=watts

ZDOL=poly(tetrafluoroethylene oxide-co-difluoromethylene oxide)α, ω diol

### BACKGROUND

Microfluidic devices developed in the early 1990s were fabricated from hard materials, such as silicon and glass, using photolithography and etching techniques. See Ouellette, J., The Industrial Physicist 2003, August/September, 14-17; Scherer, A., et al., Science 2000, 290, 1536-1539. Photolithography and etching techniques, however, are costly and labor intensive, require clean-room conditions, and pose several disadvantages from a materials standpoint. For these reasons, soft materials have emerged as alternative materials for microfluidic device fabrication. The use of soft materials has made possible the manufacture and actuation of devices containing valves, pumps, and mixers. See, e.g., Ouellette, J., The Industrial Physicist 2003, August/September, 14-17; Scherer, A., et al., Science 2000, 290, 1536-1539; Unger, M. A., et al., Science 2000, 288, 113-116; McDonald, J. C., et al., Acc. Chem. Res. 2002, 35, 491-499; and Thorsen, T., et al., Science 2002, 298, 580-584. For example, one such microfluidic device allows for control over flow direction without the use of mechanical valves. See Zhao, B., et al., Science 2001, 291, 1023-1026.

The increasing complexity of microfluidic devices has created a demand to use such devices in a rapidly growing number of applications. To this end, the use of soft materials has allowed microfluidics to develop into a useful technology that has found application in genome mapping, rapid separations, sensors, nanoscale reactions, ink-jet printing, drug delivery, Lab-on-a-Chip, in vitro diagnostics, injection nozzles, biological studies, and drug screening. See, e.g., Ouellette, J., The Industrial Physicist 2003, August/September, 14-17; Scherer, A., et al., Science 2000, 290, 1536-1539; Unger, M. A., et al., Science 2000, 288, 113-116; McDonald, J. C., et al., Acc. Chem. Res. 2002, 35, 491-499; Thorsen, T., et al., Science 2002, 298, 580-584; and Liu, J., et al., Anal. Chem. 2003, 75, 4718-4723.

Poly(dimethylsiloxane) (PDMS) is the soft material of choice for many microfluidic device applications. See Scherer, A., et al., Science 2000, 290, 1536-1539; Unger, M. A., et al., Science 2000, 288, 113-116; McDonald, J. C., et al., Acc. Chem. Res., 2002, 35, 491-499; Thorsen, T., et al., Science 2002, 298, 580-584; and Liu, J., et al., Anal. Chem. 2003, 75, 4718-4723. A PDMS material offers numerous attractive properties in microfluidic applications. Upon cross-linking, PDMS becomes an elastomeric material with a low Young's modulus, e.g., approximately 750 kPa. See Unger, M. A., et al., Science 2000, 288, 113-116. This property allows PDMS to conform to surfaces and to form reversible seals. Further, PDMS has a low surface energy, e.g., approximately 20 erg/cm², which can facilitate its release from molds after patterning. See Scherer, A., et al., Science 2000, 290, 1536-1539; McDonald, J. C., et al., Acc. Chem. Res. 2002, 35, 491-499.

Another important feature of PDMS is its outstanding gas permeability. This property allows gas bubbles within the channels of a microfluidic device to permeate out of the device. This property also is useful in sustaining cells and microorganisms inside the features of the microfluidic device. The nontoxic nature of silicones, such as PDMS, also is beneficial in this respect and allows for opportunities in the realm of medical implants. McDonald, J. C. et al., Acc. Chem. Res. 2002, 35, 491-499.

Many current PDMS microfluidic devices are based on SYLGARD® 184 (Dow Corning, Midland, Mich., United States of America). SYLGARD® 184 is cured thermally through a platinum-catalyzed hydrosilation reaction. Complete curing of SYLGARD® 184 can take as long as five hours. The synthesis of a photocurable PDMS material, however, with mechanical properties similar to that of SYLGARD® 184 for use in soft lithography recently has been reported. See Choi, K. M., et al., J. Am. Chem. Soc. 2003, 125, 4060-4061.

Despite the aforementioned advantages, PDMS suffers from a drawback in microfluidic applications in that it swells in most organic solvents. Thus, PDMS-based microfluidic devices have a limited compatibility with various organic solvents. See Lee, J. N., et al., Anal. Chem. 2003, 75, 6544-6554. Among those organic solvents that swell PDMS are hexanes, ethyl ether, toluene, dichloromethane, acetone, and acetonitrile. See Lee, J. N., et al., Anal. Chem. 2003, 75, 6544-6554. The swelling of a PDMS microfluidic device by organic solvents can disrupt its micron-scale features, e.g., a channel or plurality of channels, and can restrict or completely shut off the flow of organic solvents through the channels. Thus, microfluidic applications with a PDMS-based device are limited to the use of fluids, such as water, that do not swell PDMS. As a result, those applications that require the use of organic solvents likely will need to use microfluidic systems fabricated from hard materials, such as glass and silicon. See Lee, J. N., et al., Anal. Chem. 2003, 75, 6544-6554. This approach, however, is limited by the disadvantages of fabricating microfluidic devices from hard materials.

Moreover, PDMS-based devices and materials are notorious for not being adequately inert enough to allow them to be used even in aqueous-based chemistries. For example, PDMS is susceptible to reaction with weak and strong acids and bases. PDMS-based devices also are notorious for containing extractables, in particular extractable oligomers and cyclic siloxanes, especially after exposure to acids and bases. Because PDMS is easily swollen by organics, hydrophobic materials, even those hydrophobic materials that are slightly soluble in water, can partition into PDMS-based materials used to construct PDMS-based microfluidic devices.

Thus, an elastomeric material that exhibits the attractive mechanical properties of PDMS combined with a resistance to swelling in common organic solvents would extend the use of microfluidic devices to a variety of new chemical applications that are inaccessible by current PDMS-based devices. Accordingly, the approach demonstrated by the presently disclosed subject matter uses an elastomeric material, more particularly a functional perfluoropolyether (PFPE) material, which is resistant to swelling in common organic solvents to fabricate a microfluidic device.

Functional PFPE materials are liquids at room temperature, exhibit low surface energy, low modulus, high gas permeability, and low toxicity with the added feature of being extremely chemically resistant. See Scheirs, J., Modern Fluoropolymers; John Wiley & Sons, Ltd.: New York, 1997; pp 435-485. Further, PFPE materials exhibit hydrophobic and lyophobic properties. For this reason, PFPE materials are often used as lubricants on high-performance machinery operating in harsh conditions. The synthesis and solubility of PFPE materials in supercritical carbon dioxide has been reported. See Bunyard, W., et al., Macromolecules 1999, 32, 8224-8226. Beyond PFPEs, fluoroelastomers also can comprise fluoroolefin-based materials, including, but not limited to, copolymers of tetrafluoroethylene, hexafluoropropylene, vinylidene fluoride and alkyl vinyl ethers, often with additional cure site monomers added for crosslinking.

A PFPE microfluidic device has been previously reported by Rolland, J. et al. JACS 2004, 126, 2322-2323. The device was fabricated from a functionalized PFPE material (e.g., a PFPE dimethacrylate (MW=4,000 g/mol)) having a viscosity of the functionalized material of approximately 800 cSt. This material was end-functionalized with a free radically polymerizable methacrylate group and UV photocured free radically with a photoinitiator. In Rolland, J. et al., supra, multilayer PFPE devices were generated using a specific partial UV curing technique and the adhesion was weak and generally not strong enough for a wide range of applications. Further, the adhesion technique described by Rolland, J. et al. did not provide for adhesion to other substrates such as glass.

The presently disclosed subject matter describes the use of fluoroelastomers, especially a functional perfluoropolyether as a material for fabricating a solvent-resistant micro- and nano-scale structures, such as a microfluidic device. The use of fluoroelastomers and functional perfluoropolyethers in particular as materials for fabricating a microfluidic device addresses the problems associated with swelling in organic solvents exhibited by microfluidic devices made from other polymeric materials, such as PDMS. Accordingly, PFPE-based microfluidic devices can be used to control the flow of a small volume of a fluid, such as an organic solvent, and to perform micro- and nano-scale chemical reactions that are not amenable to other polymeric microfluidic devices.

### SUMMARY

The presently disclosed subject matter provides functional perfluoropolyether (PFPE) materials for use in fabricating microfluidic devices. In some embodiments, the presently disclosed subject matter provides a method for adhering two-dimensional and three-dimensional micro- and/or nano-scale structures, e.g., a microfluidic network, to a substrate. Further, in some embodiments, the presently disclosed subject matter provides a method for forming a hybrid microfluidic device, for example, a microfluidic device comprising a perfluoropolyether layer adhered to a second polymeric layer, wherein the second polymeric layer comprises, for example, a poly(dimethylsiloxane) layer.

The presently disclosed subject matter also provides methods for fabricating a micro- and/or nano-scale structure, e.g., a microfluidic device, by using sacrificial layers of a degradable material. More particularly, the presently disclosed subject matter provides a method for fabricating micro- and/or nano-scale structures using degradable or selectively soluble polymers as scaffolds for producing complex, two-dimensional (2-D) and three-dimensional (3-D) microfluidic networks.

Further, the presently disclosed subject matter provides functional materials for use in attaching biological and other “switchable” molecules to the interior surface of a microfluidic channel. For example, attaching a biomolecule, such as a biopolymer, to the interior surface of a microfluidic channel, provides for combinatorial peptide synthesis and/or rapid screening of enzyme-protein interactions. Further, lining a microfluidic channel with a catalyst, allows for rapid catalyst screening. Also, introduction of a switchable organic molecule into a microfluidic channel allows for the fabrication of microfluidic devices comprising hydrophilic channels and hydrophobic channels.

In some embodiments, the presently disclosed subject matter provides a method for using a functionalized perfluoropolyether network as a gas separation membrane.

Accordingly, it is an object of the presently disclosed subject matter to provide functional perfluoropolyether materials for use in fabricating and utilizing micro- and nano-scale devices, including microfluidic devices. This and other objects are achieved in whole or in part by the presently disclosed subject matter.

An object of the presently disclosed subject matter having been stated hereinabove, other aspects and objects will become evident as the description proceeds when taken in connection with the accompanying Drawings and Examples as best described herein below.

### BRIEF DESCRIPTION OF THE DRAWINGS

FIGS. 1A-1C are a series of schematic end views depicting the formation of a patterned layer of polymeric material in accordance with the presently disclosed subject matter.

FIGS. 2A-2D are a series of schematic end views depicting the formation of a microfluidic device comprising two patterned layers of a polymeric material in accordance with the presently disclosed subject matter.

FIGS. 3A-3C are schematic representations of an embodiment of the presently disclosed method for adhering a functional microfluidic device to a treated substrate.

FIGS. 4A-4C are schematic representations of an embodiment of the presently disclosed method for fabricating a multilayer microfluidic device.

FIGS. 5A and 5B are schematic representations of an embodiment of the presently disclosed method for functionalizing the interior surface of a microfluidic channel and the surface of a microtiter well.

FIG. 5A is a schematic representation of an embodiment of the presently disclosed method for functionalizing the interior surface of a microfluidic channel.

FIG. 5B is a schematic representation of an embodiment of the presently disclosed method for functionalizing the surface of a microtiter well.

FIGS. 6A-6D are schematic representations of an embodiment of the presently disclosed method for fabricating a microstructure using a degradable and/or selectively soluble material.

FIGS. 7A-7C are schematic representations of an embodiment of the presently disclosed method for fabricating complex structures in a micro- and/or nano-scale device using degradable and/or selectively soluble materials.

FIG. 8 is a schematic plan view of a microfluidic device in accordance with the presently disclosed subject matter.

FIG. 9 is a schematic of an integrated microfluidic system for biopolymer synthesis.

FIG. 10 is schematic view of a system for flowing a solution or conducting a chemical reaction in a microfluidic device in accordance with the presently disclosed subject matter. The microfluidic device 800 is depicted as a schematic plan view as shown in FIG. 8.

### DETAILED DESCRIPTION

The presently disclosed subject matter provides materials and methods for use in forming a microfluidic device and for imparting chemical functionality to a microfluidic device. In some embodiments, the presently disclosed methods comprise introducing chemical functionalities that promote and/or increase the adhesion between the layers of the microfluidic device to one another. In some embodiments, the chemical functionalities promote and/or increase the adhesion between a layer of the microfluidic device and another surface. Accordingly, in some embodiments, the presently disclosed subject matter provides a method for adhering two-dimensional and three-dimensional microfluidic networks to a substrate. In some embodiments, the presently disclosed method allows for bonding a perfluoropolyether (PFPE) material to other materials, such as a poly(dimethyl siloxane) (PDMS) material, a polyurethane material, a silicone-containing polyurethane material, and a PFPE-PDMS block copolymer material. Thus, in some embodiments, the presently disclosed subject matter provides a method for forming a hybrid microfluidic device, for example, a microfluidic device comprising a perfluoropolyether layer adhered to a polydimethylsiloxane layer, a polyurethane layer, a silicone-containing polyurethane layer, and a PFPE-PDMS block copolymer layer.

In some embodiments, the method comprises introducing a chemical functionality to the interior surface of a microfluidic channel and/or a microtiter well. In some embodiments, the introduction of a chemical functionality to the interior surface of the microfluidic channel and/or microtiter well provides for the attachment of a biopolymer and other small organic “switchable” molecules that can affect the hydrophobicity or the reactivity of the microfluidic channel and/or microtiter well.

In some embodiments, the presently disclosed subject matter provides a method for forming a micro- and/or nano-scale structure in which scaffolds of degradable or selectively soluble polymers are used to form channels, for example, inside a microfluidic device. Accordingly, the molding method disclosed herein allows for complex three-dimensional networks of microfluidic channels to be formed in a one step process.

In some embodiments, the presently disclosed subject matter provides a method for using a functionalized perfluoropolyether network as a gas separation membrane.

The presently disclosed subject matter will now be described more fully hereinafter with reference to the accompanying Drawings and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

I. Definitions

As used herein, the term “microfluidic device” generally refers to a device through which materials, particularly fluid borne materials, such as liquids, can be transported, in some embodiments on a micro-scale, and in some embodiments on a nano-scale. Thus, the microfluidic devices described by the presently disclosed subject matter can comprise microscale features, nanoscale features, and combinations thereof.

Accordingly, a microfluidic device typically comprises structural or functional features dimensioned on the order of a millimeter-scale or less, which are capable of manipulating a fluid at a flow rate on the order of a microliter/min or less. Typically, such features include, but are not limited to channels, fluid reservoirs, reaction chambers, mixing chambers, and separation regions. In some examples, the channels include at least one cross-sectional dimension that is in a range of from about 0.1 μm to about 500 μm. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps for introducing fluids, e.g., samples, reagents, buffers and the like, into the system and/or through the system; detection equipment or systems; reagent, product or data storage systems; and control systems for controlling fluid transport and/or direction within the device, monitoring and controlling environmental conditions to which fluids in the device are subjected, e.g., temperature, current, and the like.

As used herein, the term “device” includes, but is not limited to, a microfluidic device, a microtiter plate, tubing, a hose, and the like.

As used herein, the terms “channel,” “microscale channel,” and “microfluidic channel” are used interchangeably and can mean a recess or cavity formed in a material by imparting a pattern from a patterned substrate into a material or by any suitable material removing technique, or can mean a recess or cavity in combination with any suitable fluid-conducting structure mounted in the recess or cavity, such as a tube, capillary, or the like.

As used herein, the terms “flow channel” and “control channel” are used interchangeably and can mean a channel in a microfluidic device in which a material, such as a fluid, e.g., a gas or a liquid, can flow through. More particularly, the term “flow channel” refers to a channel in which a material of interest, e.g., a solvent or a chemical reagent, can flow through. Further, the term “control channel” refers to a flow channel in which a material, such as a fluid, e.g., a gas or a liquid, can flow through in such a way to actuate a valve or pump.

As used herein, the term “valve” unless otherwise indicated refers to a configuration in which two channels are separated by an elastomeric segment, e.g., a PFPE segment that can be deflected into or retracted from one of the channels, e.g., a flow channel, in response to an actuation force applied to the other channel, e.g., a control channel. The term “valve” also includes one-way valves, which comprise channels separated by a bead.

As used herein, the term “pattern” can mean a channel or a microfluidic channel or an integrated network of microfluidic channels, which, in some embodiments, can intersect at predetermined points. A pattern also can comprise one or more of a micro- or nano-scale fluid reservoir, a micro- or nano-scale reaction chamber, a micro- or nano-scale mixing chamber, and a micro- or nano-scale separation region.

As used herein, the term “intersect” can mean to meet at a point, to meet at a point and cut through or across, or to meet at a point and overlap. More particularly, as used herein, the term “intersect” describes an embodiment wherein two channels meet at a point, meet at a point and cut through or across one another, or meet at a point and overlap one another. Accordingly, in some embodiments, two channels can intersect, i.e., meet at a point or meet at a point and cut through one another, and be in fluid communication with one another. In some embodiments, two channels can intersect, i.e., meet at a point and overlap one another, and not be in fluid communication with one another, as is the case when a flow channel and a control channel intersect.

As used herein, the term “communicate” (e.g., a first component “communicates with” or “is in communication with” a second component) and grammatical variations thereof are used to indicate a structural, functional, mechanical, electrical, optical, or fluidic relationship, or any combination thereof, between two or more components or elements. As such, the fact that one component is said to communicate with a second component is not intended to exclude the possibility that additional components can be present between, and/or operatively associated or engaged with, the first and second components.

In referring to the use of a microfluidic device for handling the containment or movement of fluid, the terms “in”, “on”, “into”, “onto”, “through”, and “across” the device generally have equivalent meanings.

As used herein, the term “monolithic” refers to a structure comprising or acting as a single, uniform structure.

As used herein, the term “non-biological organic materials” refers to organic materials, i.e., those compounds having covalent carbon-carbon bonds, other than biological materials. As used herein, the term “biological materials” includes nucleic acid polymers (e.g., DNA, RNA) amino acid polymers (e.g., enzymes, proteins, and the like) and small organic compounds (e.g., steroids, hormones) wherein the small organic compounds have biological activity, especially biological activity for humans or commercially significant animals, such as pets and livestock, and where the small organic compounds are used primarily for therapeutic or diagnostic purposes. While biological materials are of interest with respect to pharmaceutical and biotechnological applications, a large number of applications involve chemical processes that are enhanced by other than biological materials, i.e., non-biological organic materials.

As used herein, the term “partial cure” refers to a process wherein less than about %100 of the polymerizable groups are reacted. Thus, the term “partially-cured material” refers to a material which has undergone a partial cure process.

As used herein, the term “full cure” refers to a process wherein about 100% of the polymerizable groups are reacted. Thus, the term “fully-cured material” refers to a material which has undergone a full cure process.

Following long-standing patent law convention, the terms “a”, “an”, and “the” refer to “one or more” when used in this application, including the claims. Thus, for example, reference to “a microfluidic channel” includes a plurality of such microfluidic channels, and so forth.

II. Materials

The presently disclosed subject matter broadly describes and employs solvent resistant, low surface energy polymeric materials, especially derived from casting liquid PFPE precursor materials onto a patterned substrate and then curing the liquid PFPE precursor materials to generate a patterned layer of functional PFPE material, which can be used to form a microfluidic device.

Representative solvent resistant elastomer-based materials include but are not limited to fluorinated elastomer-based materials. As used herein, the term “solvent resistant” refers to a material, such as an elastomeric material that neither swells nor dissolves in common hydrocarbon-based organic solvents or acidic or basic aqueous solutions. Representative fluorinated elastomer-based materials include but are not limited to perfluoropolyether (PFPE)-based materials.

Functional liquid PFPE materials exhibit desirable properties for use in a microfluidic device. For example, functional PFPE materials typically have a low surface energy (for example, about 12 mN/m); are non-toxic, UV and visible light transparent, and highly gas permeable; and cure into a tough, durable, highly fluorinated elastomeric or glassy materials with excellent release properties and resistance to swelling. The properties of these materials can be tuned over a wide range through the judicious choice of additives, fillers, reactive co-monomers, and functionalization agents. Such properties that are desirable to modify, include, but are not limited to, modulus, tear strength, surface energy, permeability, functionality, mode of cure, solubility and swelling characteristics, and the like. The non-swelling nature and easy release properties of the presently disclosed PFPE materials allow for the fabrication of microfluidic devices.

II.A. Perfluoropolyether Materials Prepared from a Liquid PFPE Precursor Material Having a Viscosity Less than about 100 Centistokes.

As would be recognized by one of ordinary skill in the art, perfluoropolyethers (PFPEs) have been in use for over 25 years for many applications. Commercial PFPE materials are made by polymerization of perfluorinated monomers. The first member of this class was made by the cesium fluoride catalyzed polymerization of hexafluoropropene oxide (HFPO) yielding a series of branched polymers designated as KRYTOX® (DuPont, Wilmington, Del., United States of America). A similar polymer is produced by the UV catalyzed photo-oxidation of hexafluoropropene (FOMBLIN® Y) (Solvay Solexis, Brussels, Belgium). Further, a linear polymer (FOMBLIN® Z) (Solvay) is prepared by a similar process, but utilizing tetrafluoroethylene. Finally, a fourth polymer (DEMNUM®) (Daikin Industries, Ltd., Osaka, Japan) is produced by polymerization of tetrafluorooxetane followed by direct fluorination. Structures for these fluids are presented in Table I. Table II contains property data for some members of the PFPE class of lubricants. Likewise, the physical properties of functional PFPEs are provided in Table III. In addition to these commercially available PFPE fluids, a new series of structures are being prepared by direct fluorination technology. Representative structures of these new PFPE materials appear in Table IV. Of the abovementioned PFPE fluids, only KRYTOX® and FOMBLIN® Z have been extensively used in applications. See Jones, W. R., Jr., The Properties of Perfluoropolyethers Used for Space Applications, NASA Technical Memorandum 106275 (July 1993), which is incorporated herein by reference in its entirety. Accordingly, the use of such PFPE materials is provided in the presently disclosed subject matter.

TABLE I
Names and Chemical Structures of Commercial PFPE Fluids

| Name | Structure |
|---|---|
| DEMNUM ® | C₃F₇O(CF₂CF₂CF₂O)ₓC₂F₅ |
| KRYTOX ® | C₃F₇O[CF(CF₃)CF₂O]ₓC₂F₅ |
| FOMBLIN ® Y | C₃F₇O[CF(CF₃)CF₂O]ₓ(CF₂O)_{y}C₂F₅ |
| FOMBLIN ® Z | CF₃O(CF₂CF₂O)ₓ(CF₂O)_{y}CF₃ |

TABLE II
PFPE Physical Properties

| | Average | Viscosity | | Pour | Vapor Pressure, | |
|---|---|---|---|---|---|---|
| | Molecular | at 20° C., | Viscosity | Point, | Torr | |
| Lubricant | Weight | (cSt) | Index | ° C. | 20° C. | 100° C. |
| FOMBLIN ® Z-25 | 9500 | 255 | 355 | −66 | 2.0 × 10⁻¹² | 1 × 10⁻⁸ |
| KRYTOX ® 143AB | 3700 | 230 | 113 | −40 | 1.5 × 10⁻⁶ | 3 × 10⁻⁴ |
| KRYTOX ® 143AC | 6250 | 800 | 134 | −35 | 2 × 10⁻⁸ | 8 × 10⁻⁶ |
| DEMNUM ® S-200 | 8400 | 500 | 210 | −53 | 1 × 10⁻¹⁰ | 1 × 10⁻⁷ |

TABLE III
PFPE Physical Properties of Functional PFPEs

| | Average | Viscosity | | |
|---|---|---|---|---|
| | Molecular | at 20° C., | Vapor Pressure, Torr | |
| Lubricant | Weight | (cSt) | 20° C. | 100° C. |
| FOMBLIN ® | 2000 | 85 | 2.0 × 10⁻⁵ | 2.0 × 10⁻⁵ |
| Z-DOL 2000 | | | | |
| FOMBLIN ® | 2500 | 76 | 1.0 × 10⁻⁷ | 1.0 × 10⁻⁴ |
| Z-DOL 2500 | | | | |
| FOMBLIN ® | 4000 | 100 | 1.0 × 10⁻⁸ | 1.0 × 10⁻⁴ |
| Z-DOL 4000 | | | | |
| FOMBLIN ® | 500 | 2000 | 5.0 × 10⁻⁷ | 2.0 × 10⁻⁴ |
| Z-TETROL | | | | |

TABLE IV
Names and Chemical Structures of Representative PFPE Fluids

| Name | Structure^{a} |
|---|---|
| Perfluoropoly(methylene oxide) (PMO) | CF₃O(CF₂O)ₓCF₃ |
| Perfluoropoly(ethylene oxide) (PEO) | CF₃O(CF₂CF₂O)ₓCF₃ |
| Perfluoropoly(dioxolane) (DIOX) | CF₃O(CF₂CF₂OCF₂O)ₓCF₃ |
| Perfluoropoly(trioxocane) (TRIOX) | CF₃O[(CF₂CF₂O)₂CF₂O]ₓCF₃ |

^{a}wherein x is any integer.

In some embodiments, the perfluoropolyether precursor comprises poly(tetrafluoroethylene oxide-co-difluoromethylene oxide)α,ω diol, which in some embodiments can be photocured to form one of a perfluoropolyether dimethacrylate and a perfluoropolyether distyrenic compound. A representative scheme for the synthesis and photocuring of a functionalized perfluoropolyether is provided in Scheme 1.

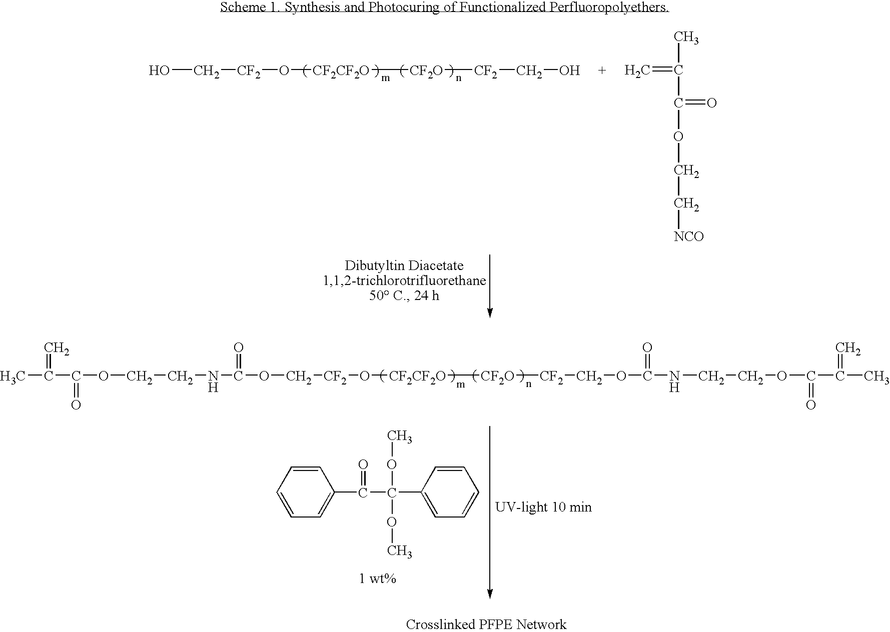

II.B. Perfluoropolyether Materials Prepared from a Liquid PFPE Precursor Material Having a Viscosity Greater than about 100 Centistokes.

The methods provided herein below for promoting and/or increasing adhesion between a layer of a PFPE material and another material and/or a substrate and for adding a chemical functionality to a surface comprise a PFPE material having a characteristic selected from the group consisting of a viscosity greater than about 100 centistokes (cSt) and a viscosity less than about 100 cSt, provided that the liquid PFPE precursor material having a viscosity less than 100 cSt is not a free-radically photocurable PFPE material. As provided herein, the viscosity of a liquid PFPE precursor material refers to the viscosity of that material prior to functionalization, e.g., functionalization with a methacrylate or a styrenic group.

Thus, in some embodiments, PFPE material is prepared from a liquid PFPE precursor material having a viscosity greater than about 100 centistokes (cSt). In some embodiments, the liquid PFPE precursor is end-capped with a polymerizable group. In some embodiments, the polymerizable group is selected from the group consisting of an acrylate, a methacrylate, an epoxy, an amino, a carboxylic, an anhydride, a maleimide, an isocyanato, an olefinic, and a styrenic group.

In some embodiments, the perfluoropolyether material comprises a backbone structure selected from the group consisting of: 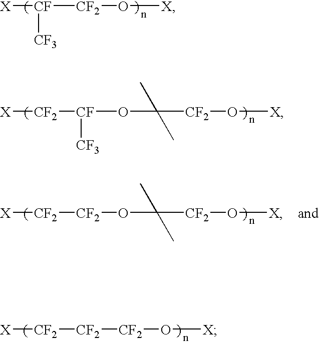
wherein X is present or absent, and when present comprises an endcapping group, and n is an integer from 1 to 100.

In some embodiments, the PFPE liquid precursor is synthesized from hexafluoropropylene oxide as shown in Scheme 2.

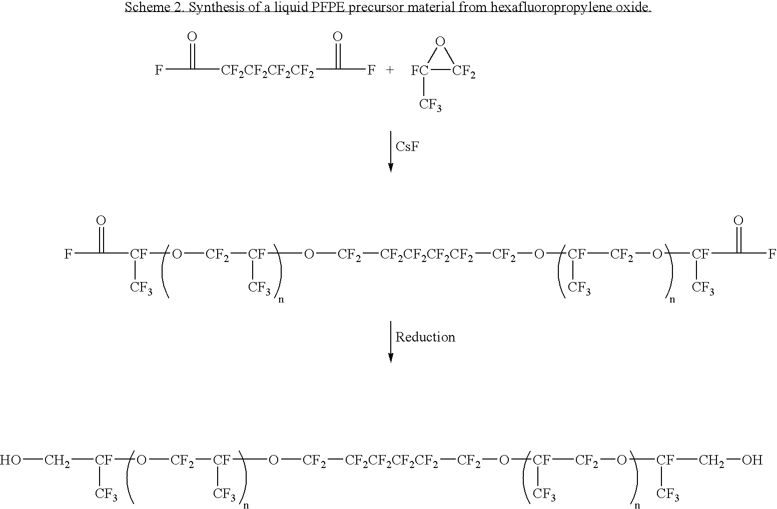

Is some embodiments, the liquid PFPE precursor is synthesized from hexafluoropropylene oxide as shown in Scheme 3.

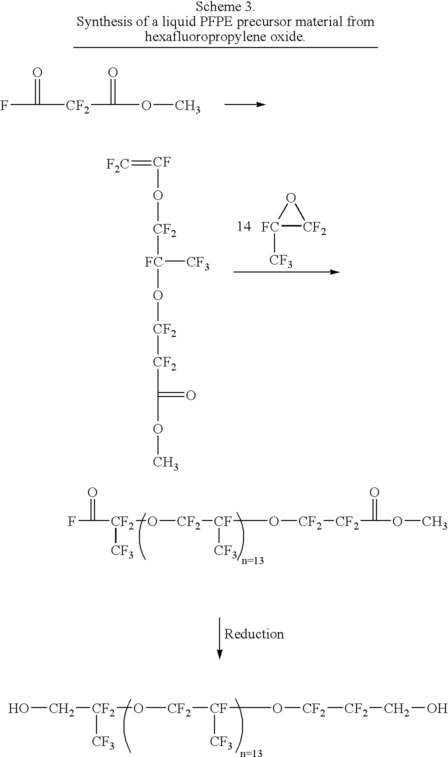

In some embodiments the liquid PFPE precursor comprises a chain extended material such that two or more chains are linked together before adding polymerizablable groups. Accordingly, in some embodiments, a “linker group” joins two chains to one molecule. In some embodiments, as shown in Scheme 4, the linker group joins three or more chains.

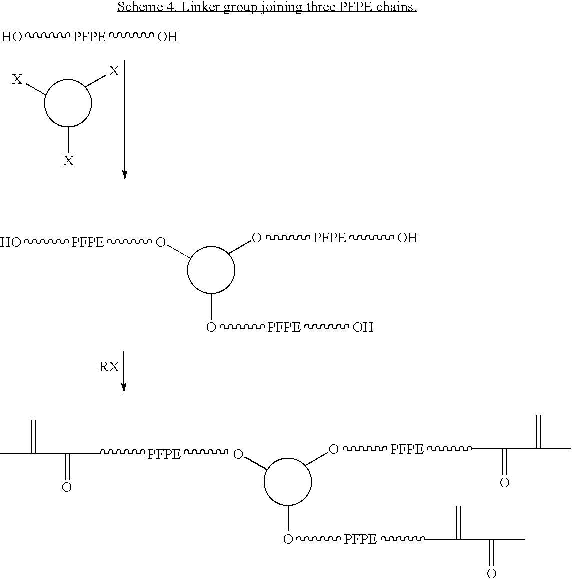

In some embodiments, X is selected from the group consisting of an isocyanate, an acid chloride, an epoxy, and a halogen. In some embodiments, R is selected from the group consisting of an acrylate, a methacrylate, a styrene, an epoxy, a carboxylic, an anhydride, a maleimide, an isocyanate, an olefinic, and an amine. In some embodiments, the circle represents any multifunctional molecule. In some embodiments, the multifunctional molecule comprises a cyclic molecule. PFPE refers to any PFPE material provided hereinabove.

In some embodiments, the liquid PFPE precursor comprises a hyperbranched polymer as provided in Scheme 5, wherein PFPE refers to any PFPE material provided hereinabove.

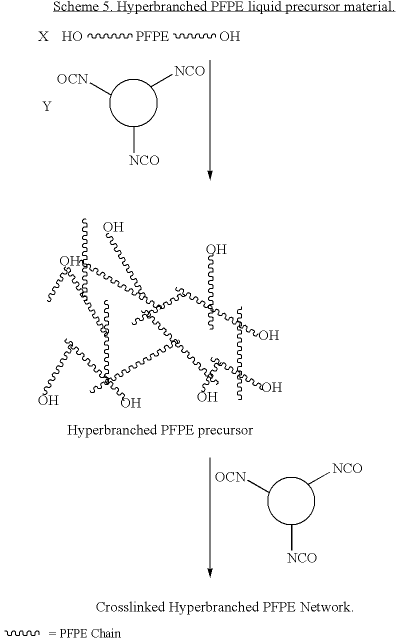

In some embodiments, the liquid PFPE material comprises an end-functionalized material selected from the group consisting of:

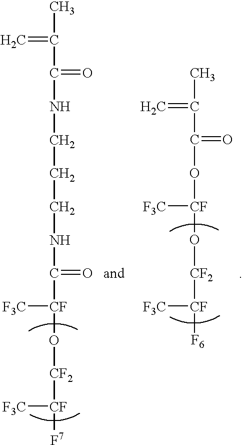

In some embodiments the PFPE liquid precursor is encapped with an epoxy moiety that can be photocured using a photoacid generator. Photoacid generators suitable for use in the presently disclosed subject matter include, but are not limited to: bis(4-tert-butylphenyl)iodonium p-toluenesulfonate, bis(4-tert-butylphenyl)iodonium triflate, (4-bromophenyl)diphenylsulfonium triflate, (tert-butoxycarbonylmethoxynaphthyl)-diphenylsulfonium triflate, (tert-butoxycarbonylmethoxyphenyl)diphenylsulfonium triflate, (4-tert-butylphenyl)diphenylsulfonium triflate, (4-chlorophenyl)diphenylsulfonium triflate, diphenyliodonium-9,10-dimethoxyanthracene-2-sulfonate, diphenyliodonium hexafluorophosphate, diphenyliodonium nitrate, diphenyliodonium perfluoro-1-butanesulfonate, diphenyliodonium p-toluenesulfonate, diphenyliodonium triflate, (4-fluorophenyl)diphenylsulfonium triflate, N-hydroxynaphthalimide triflate, N-hydroxy-5-norbornene-2,3-dicarboximide perfluoro-1-butanesulfonate, N-hydroxyphthalimide triflate, [4-[(2-hydroxytetradecyl)oxy]phenyl]phenyliodonium hexafluoroantimonate, (4-iodophenyl)diphenylsulfonium triflate, (4-methoxyphenyl)diphenylsulfonium triflate, 2-(4-methoxystyryl)-4,6-bis(trichloromethyl)-1,3,5-triazine, (4-methylphenyl)diphenylsulfonium triflate, (4-methylthiophenyl)methyl phenyl sulfonium triflate, 2-naphthyl diphenylsulfonium triflate, (4-phenoxyphenyl)diphenylsulfonium triflate, (4-phenylthiophenyl)diphenylsulfonium triflate, thiobis(triphenyl sulfonium hexafluorophosphate), triarylsulfonium hexafluoroantimonate salts, triarylsulfonium hexafluorophosphate salts, triphenylsulfonium perfluoro-1-butanesulfonate, triphenylsulfonium triflate, tris(4-tert-butylphenyl)sulfonium perfluoro-1-butanesulfonate, and tris(4-tert-butylphenyl)sulfonium triflate.

In some embodiments the liquid PFPE precursor cures into a highly UV and/or highly visible light transparent elastomer. In some embodiments the liquid PFPE precursor cures into an elastomer that is highly permeable to oxygen, carbon dioxide, and nitrogen, a property that can facilitate maintaining the viability of biological fluids/cells disposed therein. In some embodiments, additives are added or layers are created to enhance the barrier properties of the device to molecules, such as oxygen, carbon dioxide, nitrogen, dyes, reagents, and the like.

In some embodiments, the material suitable for use with the presently disclosed subject matter comprises a silicone material comprising a fluoroalkyl functionalized polydimethylsiloxane (PDMS) having the following structure: 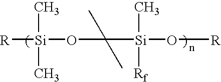
wherein:

R is selected from the group consisting of an acrylate, a methacrylate, and a vinyl group;

R_{f} comprises a fluoroalkyl chain; and

n is an integer from 1 to 100,000.

In some embodiments, the material suitable for use with the presently disclosed subject matter comprises a styrenic material comprising a fluorinated styrene monomer selected from the group consisting of: 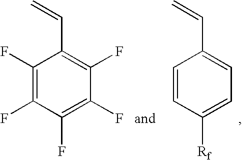
wherein R_{f} comprises a fluoroalkyl chain.

In some embodiments, the material suitable for use with the presently disclosed subject matter comprises an acrylate material comprising a fluorinated acrylate or a fluorinated methacrylate having the following structure: 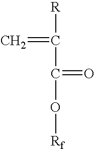
wherein:

R is selected from the group consisting of H, alkyl, substituted alkyl, aryl, and substituted aryl; and

R_{f} comprises a fluoroalkyl chain with a —CH₂— or a —CH₂—CH₂— spacer between a perfluoroalkyl chain and the ester linkage. In some embodiments, the perfluoroalkyl group has hydrogen substituents.

In some embodiments, the material suitable for use with the presently disclosed subject matter comprises a triazine fluoropolymer comprising a fluorinated monomer.

In some embodiments, the fluorinated monomer or fluorinated oligomer that can be polymerized or crosslinked by a metathesis polymerization reaction comprises a functionalized olefin. In some embodiments, the functionalized olefin comprises a functionalized cyclic olefin.

II.C. Fluoroolefin-Based Materials

Further, in some embodiments, the materials used herein are selected from highly fluorinated fluoroelastomers, e.g., fluoroelastomers comprising at least fifty-eight weight percent fluorine, as described in U.S. Pat. No. 6,512,063 to Tang, which is incorporated herein by reference in its entirety. Such fluoroelastomers can be partially fluorinated or perfluorinated and can contain between 25 to 70 weight percent, based on the weight of the fluoroelastomer, of copolymerized units of a first monomer, e.g., vinylidene fluoride (VF₂) or tetrafluoroethylene (TFE). The remaining units of the fluoroelastomers comprise one or more additional copolymerized monomers, which are different from the first monomer, and are selected from the group consisting of fluorine-containing olefins, fluorine containing vinyl ethers, hydrocarbon olefins, and combinations thereof.

These fluoroelastomers include VITON® (DuPont Dow Elastomers, Wilmington, Del., United States of America) and Kel-F type polymers, as described for microfluidic applications in U.S. Pat. No. 6,408,878 to Unger et al. These commercially available polymers, however, have Mooney viscosities ranging from about 40 to 65 (ML 1+10 at 121° C.) giving them a tacky, gum-like viscosity. When cured, they become a stiff, opaque solid. As currently available, VITON® and Kel-F have limited utility for micro-scale molding. Curable species of similar compositions, but having lower viscosity and greater optical clarity, is needed in the art for the applications described herein. A lower viscosity (e.g., 2 to 32 (ML 1+10 at 121° C.)) or more preferably as low as 80 to 2000 cSt at 20° C., composition yields a pourable liquid with a more efficient cure.

More particularly, the fluorine-containing olefins include, but are not limited to, vinylidine fluoride, hexafluoropropylene (HFP), tetrafluoroethylene (TFE), 1,2,3,3,3-pentafluoropropene (1-HPFP), chlorotrifluoroethylene (CTFE) and vinyl fluoride.

The fluorine-containing vinyl ethers include, but are not limited to perfluoro(alkyl vinyl)ethers (PAVEs). More particularly, perfluoro(alkyl vinyl)ethers for use as monomers include perfluoro(alkyl vinyl)ethers of the following formula:
CF₂═CFO(R_{f}O)ₙ(R_{f}O)ₘR_{f}
wherein each R_{f} is independently a linear or branched C₁-C₆ perfluoroalkylene group, and m and n are each independently an integer from 0 to 10.

In some embodiments, the perfluoro(alkyl vinyl)ether comprises a monomer of the following formula:
CF₂═CFO(CF₂CFXO)ₙR_{f}
wherein X is F or CF₃, n is an integer from 0 to 5, and R_{f} is a linear or branched C₁-C₆ perfluoroalkylene group. In some embodiments, n is 0 or 1 and R_{f} comprises 1 to 3 carbon atoms. Representative examples of such perfluoro(alkyl vinyl)ethers include perfluoro(methyl vinyl)ether (PMVE) and perfluoro(propyl vinyl)ether (PPVE).

In some embodiments, the perfluoro(alkyl vinyl)ether comprises a monomer of the following formula:
CF₂═CFO[(CF₂)ₘCF₂CFZO)ₙR_{f}
wherein R_{f} is a perfluoroalkyl group having 1-6 carbon atoms, m is an integer from 0 or 1, n is an integer from 0 to 5, and Z is F or CF₃. In some embodiments, R_{f} is C₃F₇, m is 0, and n is 1.

In some embodiments, the perfluoro(alkyl vinyl)ether monomers include compounds of the formula:
CF₂═CFO[(CF₂CF{CF₃}O)ₙ(CF₂CF₂CF₂O)ₘ(CF2)ₚ]CₓF₂ₓ₊₁
wherein m and n each integers independently from 0 to 10, p is an integer from 0 to 3, and x is an integer from 1 to 5. In some embodiments, n is 0 or 1, m is 0 or 1,and x is 1.

Other examples of useful perfluoro(alkyl vinyl ethers) include:
CF₂═CFOCF₂CF(CF₃)O(CF₂O)ₘCₙF₂ₙ₊₁
wherein n is an integer from 1 to 5, m is an integer from 1 to 3. In some embodiments, n is 1.

In embodiments wherein copolymerized units of a perfluoro(alkyl vinyl)ether (PAVE) are present in the presently described fluoroelastomers, the PAVE content generally ranges from 25 to 75 weight percent, based on the total weight of the fluoroelastomer. If the PAVE is perfluoro(methyl vinyl)ether (PMVE), then the fluoroelastomer contains between 30 and 55 wt. % copolymerized PMVE units.

Hydrocarbon olefins useful in the presently described fluoroelastomers include, but are not limited to ethylene (E) and propylene (P). In embodiments wherein copolymerized units of a hydrocarbon olefin are present in the presently described fluoroelastomers, the hydrocarbon olefin content is generally 4 to 30 weight percent.

Further, the presently described fluoroelastomers can, in some embodiments, comprise units of one or more cure site monomers. Examples of suitable cure site monomers include: i) bromine-containing olefins; ii) iodine-containing olefins; iii) bromine-containing vinyl ethers; iv) iodine-containing vinyl ethers; v) fluorine-containing olefins having a nitrile group; vi) fluorine-containing vinyl ethers having a nitrile group; vii) 1,1,3,3,3-pentafluoropropene (2-HPFP); viii) perfluoro(2-phenoxypropyl vinyl)ether; and ix) non-conjugated dienes.

The brominated cure site monomers can contain other halogens, preferably fluorine. Examples of brominated olefin cure site monomers are CF₂═CFOCF₂CF₂CF₂OCF₂CF₂Br; bromotrifluoroethylene; 4-bromo-3,3,4,4-tetrafluorobutene-1 (BTFB); and others such as vinyl bromide, 1-bromo-2,2-difluoroethylene; perfluoroallyl bromide; 4-bromo-1,1,2-trifluorobutene-1; 4-bromo-1,1,3,3,4,4,-hexafluorobutene; 4-bromo-3-chloro-1,1,3,4,4-pentafluorobutene; 6-bromo-5,5,6,6-tetrafluorohexene; 4-bromoperfluorobutene-1 and 3,3-difluoroallyl bromide. Brominated vinyl ether cure site monomers include 2-bromo-perfluoroethyl perfluorovinyl ether and fluorinated compounds of the class CF₂Br—R_{f}—O—CF═CF₂ (wherein R_{f} is a perfluoroalkylene group), such as CF₂BrCF₂O—CF═CF₂, and fluorovinyl ethers of the class ROCF═CFBr or ROCBr═CF₂ (wherein R is a lower alkyl group or fluoroalkyl group), such as CH₃OCF═CFBr or CF₃CH₂OCF═CFBr.

Suitable iodinated cure site monomers include iodinated olefins of the formula: CHR═CH-Z-CH₂CHR—I, wherein R is —H or —CH₃; Z is a C₁ to C₁₈ (per)fluoroalkylene radical, linear or branched, optionally containing one or more ether oxygen atoms, or a (per)fluoropolyoxyalkylene radical as disclosed in U.S. Pat. No. 5,674,959. Other examples of useful iodinated cure site monomers are unsaturated ethers of the formula: I(CH₂CF₂CF₂)ₙOCF═CF₂ and ICH₂CF₂O[CF(CF₃)CF₂O]ₙCF═CF₂, and the like, wherein n is an integer from 1 to 3, such as disclosed in U.S. Pat. No. 5,717,036. In addition, suitable iodinated cure site monomers including iodoethylene, 4-iodo-3,3,4,4-tetrafluorobutene-1 (ITFB); 3-chloro-4-iodo-3,4,4-trifluorobutene; 2-iodo-1,1,2,2-tetrafluoro-1-(vinyloxy)ethane; 2-iodo-1-(perfluorovinyloxy)-1, I,-2,2-tetrafluoroethylene; 1,1,2,3,3,3-hexafluoro-2-iodo-1-(perfluorovinyloxy)propane; 2-iodoethyl vinyl ether; 3,3,4,5,5,5-hexafluoro-4-iodopentene; and iodotrifluoroethylene are disclosed in U.S. Pat. No. 4,694,045. Allyl iodide and 2-iodo-perfluoroethyl perfluorovinyl ether also are useful cure site monomers.

Useful nitrile-containing cure site monomers include those of the formulas shown below:
CF₂═CF—O(CF₂)ₙ—CN
wherein n is an integer from 2 to 12. In some embodiments, n is an integer from 2 to 6.
CF₂═CF—O[CF₂—CF(CF)—O]ₙ—CF₂—CF(CF₃)—CN
wherein n is an integer from 0 to 4. In some embodiments, n is an integer from 0 to 2.
CF₂═CF—[OCF₂CF(CF₃)]—O—(CF₂)ₙ—CN
wherein x is 1 or 2, and n is an integer from 1 to 4; and
CF₂═CF—O—(CF₂)ₙ—CF(CF₃)—CN
wherein n is an integer from 2 to 4. In some embodiments, the cure site monomers are perfluorinated polyethers having a nitrile group and a trifluorovinyl ether group.

In some embodiments, the cure site monomer is:
CF₂═CFOCF₂CF(CF₃)OCF₂CF₂CN
i.e., perfluoro(8-cyano-5-methyl-3,6-dioxa-1-octene) or 8-CNVE.

Examples of non-conjugated diene cure site monomers include, but are not limited to 1,4-pentadiene; 1,5-hexadiene; 1,7-octadiene; 3,3,4,4-tetrafluoro-1,5-hexadiene; and others, such as those disclosed in Canadian Patent No. 2,067,891 and European Patent No. 0784064A1. A suitable triene is 8-methyl-4-ethylidene-1,7-octadiene.

In embodiments wherein the fluoroelastomer will be cured with peroxide, the cure site monomer is preferably selected from the group consisting of 4-bromo-3,3,4,4-tetrafluorobutene-1 (BTFB); 4-iodo-3,3,4,4-tetrafluorobutene-1 (ITFB); allyl iodide; bromotrifluoroethylene and 8-CNVE. In embodiments wherein the fluoroelastomer will be cured with a polyol, 2-HPFP or perfluoro(2-phenoxypropyl vinyl)ether is the preferred cure site monomer. In embodiments wherein the fluoroelastomer will be cured with a tetraamine, bis(aminophenol) or bis(thioaminophenol), 8-CNVE is the preferred cure site monomer.

Units of cure site monomer, when present in the presently disclosed fluoroelastomers, are typically present at a level of 0.05-10 wt. % (based on the total weight of fluoroelastomer), preferably 0.05-5 wt. % and most preferably between 0.05 and 3 wt. %.

Fluoroelastomers which can be used in the presently disclosed subject matter include, but are not limited to, those having at least 58 wt. % fluorine and comprising copolymerized units of i) vinylidene fluoride and hexafluoropropylene; ii) vinylidene fluoride, hexafluoropropylene and tetrafluoroethylene; iii) vinylidene fluoride, hexafluoropropylene, tetrafluoroethylene and 4-bromo-3,3,4,4-tetrafluorobutene-1; iv) vinylidene fluoride, hexafluoropropylene, tetrafluoroethylene and 4-iodo-3,3,4,4-tetrafluorobutene-1; v) vinylidene fluoride, perfluoro(methyl vinyl)ether, tetrafluoroethylene and 4-bromo-3,3,4,4-tetrafluorobutene-1; vi) vinylidene fluoride, perfluoro(methyl vinyl)ether, tetrafluoroethylene and 4-iodo-3,3,4,4-tetrafluorobutene-1; vii) vinylidene fluoride, perfluoro(methyl vinyl)ether, tetrafluoroethylene and 1,1,3,3,3-pentafluoropropene; viii) tetrafluoroethylene, perfluoro(methyl vinyl)ether and ethylene; ix) tetrafluoroethylene, perfluoro(methyl vinyl)ether, ethylene and 4-bromo-3,3,4,4-tetrafluorobutene-1; x) tetrafluoroethylene, perfluoro(methyl vinyl)ether, ethylene and 4-iodo-3,3,4,4-tetrafluorobutene-1; xi) tetrafluoroethylene, propylene and vinylidene fluoride; xii) tetrafluoroethylene and perfluoro(methyl vinyl)ether; xiii) tetrafluoroethylene, perfluoro(methyl vinyl)ether and perfluoro(8-cyano-5-methyl-3,6-dioxa-1-octene); xiv) tetrafluoroethylene, perfluoro(methyl vinyl)ether and 4-bromo-3,3,4,4-tetrafluorobutene-1; xv) tetrafluoroethylene, perfluoro(methyl vinyl)ether and 4-iodo-3,3,4,4-tetrafluorobutene-1; and xvi) tetrafluoroethylene, perfluoro(methyl vinyl)ether and perfluoro(2-phenoxypropyl vinyl)ether.

Additionally, iodine-containing endgroups, bromine-containing endgroups or combinations thereof can optionally be present at one or both of the fluoroelastomer polymer chain ends as a result of the use of chain transfer or molecular weight regulating agents during preparation of the fluoroelastomers. The amount of chain transfer agent, when employed, is calculated to result in an iodine or bromine level in the fluoroelastomer in the range of 0.005-5 wt. %, preferably 0.05-3 wt. %.

Examples of chain transfer agents include iodine-containing compounds that result in incorporation of bound iodine at one or both ends of the polymer molecules. Methylene iodide; 1,4-diiodoperfluoro-n-butane; and 1,6-diiodo-3,3,4,4-tetrafluorohexane are representative of such agents. Other iodinated chain transfer agents include 1,3-diiodoperfluoropropane; 1,6-diiodoperfluorohexane; 1,3-diiodo-2-chloroperfluoropropane; 1,2-di(iododifluoromethyl)perfluorocyclobutane; monoiodoperfluoroethane; monoiodoperfluorobutane; 2-iodo-1-hydroperfluoroethane, and the like. Also included are the cyano-iodine chain transfer agents disclosed European Patent No. 0868447A1. Particularly preferred are diiodinated chain transfer agents.

Examples of brominated chain transfer agents include 1-bromo-2-iodoperfluoroethane; 1-bromo-3-iodoperfluoropropane; 1-iodo-2-bromo-1,1-difluoroethane and others such as disclosed in U.S. Pat. No. 5,151,492.

Other chain transfer agents suitable for use include those disclosed in U.S. Pat. No. 3,707,529. Examples of such agents include isopropanol, diethylmalonate, ethyl acetate, carbon tetrachloride, acetone and dodecyl mercaptan.

III. Method for Forming a Microfluidic Device Through a Thermal Free Radical Curing Process

In some embodiments, the presently disclosed subject matter provides a method for forming a microfluidic device by which a functional liquid perfluoropolyether (PFPE) precursor material is contacted with a patterned substrate, i.e., a master, and is thermally cured using a free radical initiator. As provided in more detail herein below, in some embodiments, the liquid PFPE precursor material is fully cured to form a fully cured PFPE network, which can then be removed from the patterned substrate and contacted with a second substrate to form a reversible, hermetic seal.

In some embodiments, the liquid PFPE precursor material is partially cured to form a partially cured PFPE network. In some embodiments, the partially cured network is contacted with a second partially cured layer of PFPE material and the curing reaction is taken to completion, thereby forming a permanent bond between the PFPE layers.

Further, the partially cured PFPE network can be contacted with a layer or substrate comprising another polymeric material, such as poly(dimethylsiloxane) or another polymer, and then thermally cured so that the PFPE network adheres to the other polymeric material. Additionally, the partially cured PFPE network can be contacted with a solid substrate, such as glass, quartz, or silicon, and then bonded to the substrate through the use of a silane coupling agent.

III.A. Method of Forming a Patterned Layer of an Elastomeric Material

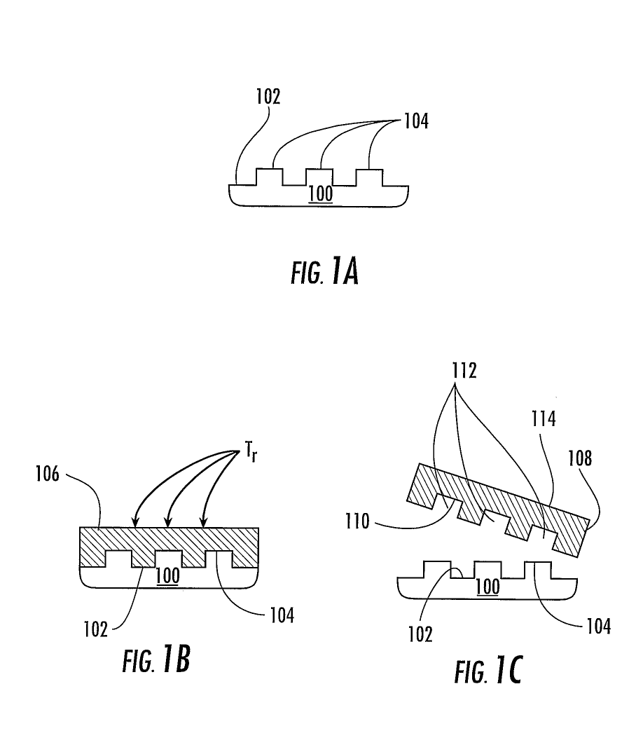

In some embodiments, the presently disclosed subject matter provides a method of forming a patterned layer of an elastomeric material. The presently disclosed method is suitable for use with the perfluoropolyether material described in Sections II.A. and II.B., and the fluoroolefin-based materials described in Section II.C. An advantage of using a higher viscosity PFPE material allows, among other things, for a higher molecular weight between cross links. A higher molecular weight between cross links can improve the elastomeric properties of the material, which can prevent among other things, cracks from forming. Referring now to FIGS. 1A-1C, a schematic representation of an embodiment of the presently disclosed subject matter is shown. A substrate 100 having a patterned surface 102 comprising a raised protrusion 104 is depicted. Accordingly, the patterned surface 102 of the substrate 100 comprises at least one raised protrusion 104, which forms the shape of a pattern. In some embodiments, patterned surface 102 of substrate 100 comprises a plurality of raised protrusions 104 which form a complex pattern.

As best seen in FIG. 1B, a liquid precursor material 106 is disposed on patterned surface 102 of substrate 100. As shown in FIG. 1B, the liquid precursor material 102 is treated with a treating process Tᵣ. Upon the treating of liquid precursor material 106, a patterned layer 108 of an elastomeric material (as shown in FIG. 1 C) is formed.

As shown in FIG. 1C, the patterned layer 108 of the elastomeric material comprises a recess 110 that is formed in the bottom surface of the patterned layer 108. The dimensions of recess 110 correspond to the dimensions of the raised protrusion 104 of patterned surface 102 of substrate 100. In some embodiments, recess 110 comprises at least one channel 112, which in some embodiments of the presently disclosed subject matter comprises a microscale channel. Patterned layer 108 is removed from patterned surface 102 of substrate 100 to yield microfluidic device 114.

In some embodiments, the patterned substrate comprises an etched silicon wafer. In some embodiments, the patterned substrate comprises a photoresist patterned substrate. For the purposes of the presently disclosed subject matter, the patterned substrate can be fabricated by any of the processing methods known in the art, including, but not limited to, photolithography, electron beam lithography, and ion milling.

In some embodiments, the patterned layer of perfluoropolyether is between about 0.1 micrometers and about 100 micrometers thick. In some embodiments, the patterned layer of perfluoropolyether is between about 0.1 millimeters and about 10 millimeters thick. In some embodiments, the patterned layer of perfluoropolyether is between about one micrometer and about 50 micrometers thick. In some embodiments, the patterned layer of perfluoropolyether is about 20 micrometers thick. In some embodiments, the patterned layer of perfluoropolyether is about 5 millimeters thick.

In some embodiments, the patterned layer of perfluoropolyether comprises a plurality of microscale channels. In some embodiments, the channels have a width ranging from about 0.01 μm to about 1000 μm; a width ranging from about 0.05 μm to about 1000 μm; and/or a width ranging from about 1 μm to about 1000 μm. In some embodiments, the channels have a width ranging from about 1 μm to about 500 μm; a width ranging from about 1 μm to about 250 μm; and/or a width ranging from about 10 μm to about 200 μm. Exemplary channel widths include, but are not limited to, 0.1 μm, 1 μm, 2 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, and 250 μm.

In some embodiments, the channels have a depth ranging from about 1 μm to about 1000 μm; and/or a depth ranging from about 1 μm to 100 μm. In some embodiments, the channels have a depth ranging from about 0.01 μm to about 1000 μm; a depth ranging from about 0.05 μm to about 500 μm; a depth ranging from about 0.2 μm to about 250 μm; a depth ranging from about 1 μm to about 100 μm; a depth ranging from about 2 μm to about 20 μm; and/or a depth ranging from about 5 μm to about 10 μm. Exemplary channel depths include, but are not limited to, 0.01 μm, 0.02 μm, 0.05 μm, 0.1 μm, 0.2 μm, 0.5 μm, 1 μm, 2 μm, 3 μm, 4 μm, 5 μm, 7.5 μm, 10 μm, 12.5 μm, 15 μm, 17.5 μm, 20 μm, 22.5 μm, 25 μm, 30 μm, 40 μm, 50 μm, 75 μm, 100 μm, 150 μm, 200 μm, and 250 μm.

In some embodiments, the channels have a width-to-depth ratio ranging from about 0.1:1 to about 100:1. In some embodiments, the channels have a width-to-depth ratio ranging from about 1:1 to about 50:1. In some embodiments, the channels have a width-to-depth ratio ranging from about 2:1 to about 20:1. In some embodiments, the channels have a width-to-depth ratio ranging from about 3:1 to about 15:1. In some embodiments, the channels have a width-to-depth ratio of about 10:1.

One of ordinary skill in the art would recognize that the dimensions of the channels of the presently disclosed subject matter are not limited to the exemplary ranges described hereinabove and can vary in width and depth to affect the magnitude of force required to flow a material through the channel and/or to actuate a valve to control the flow of the material therein. Further, as will be described in more detail herein below, channels of greater width are contemplated for use as a fluid reservoir, a reaction chamber, a mixing channel, a separation region, and the like.

III.B. Method for Forming a Multilayer Patterned Material

In some embodiments, the presently disclosed subject matter describes a method for forming a multilayer patterned material, e.g., a multilayer patterned PFPE material. In some embodiments, the multilayer patterned perfluoropolyether material is used to fabricate a monolithic PFPE-based microfluidic device.

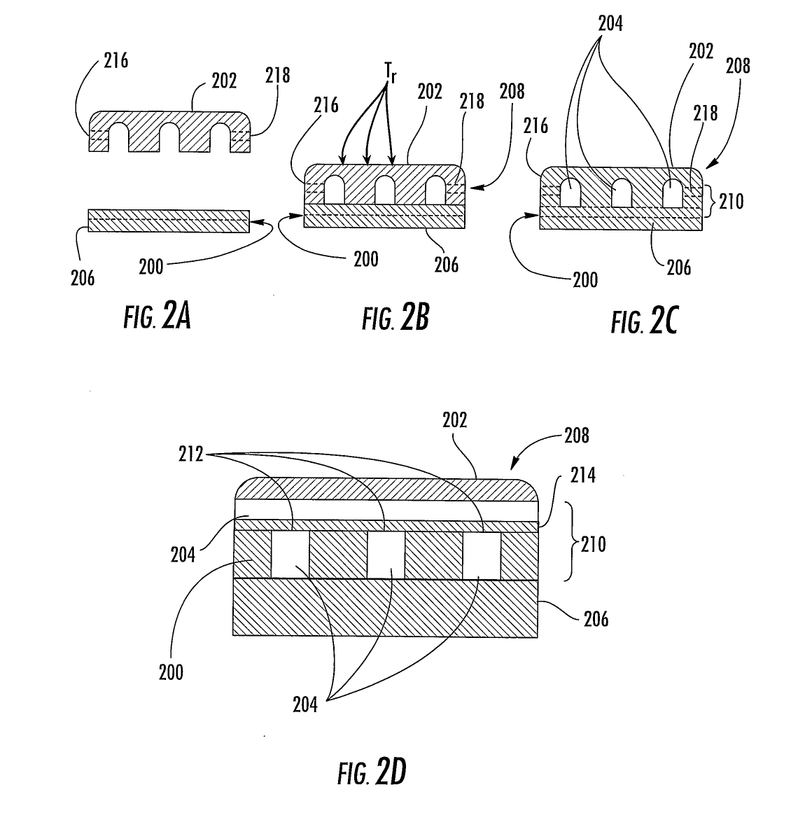

Referring now to FIGS. 2A-2D, a schematic representation of the preparation of an embodiment of the presently disclosed subject matter is shown. Patterned layers 200 and 202 are provided, each of which, in some embodiments, comprise a perfluoropolyether material prepared from a liquid PFPE precursor material having a viscosity greater than about 100 cSt. In this example, each of the patterned layers 200 and 202 comprise a plurality of channels 204. In this embodiment of the presently disclosed subject matter, the plurality of channels 204 comprise microscale channels. In patterned layer 200, the channels are represented by a dashed line, i.e., in shadow, in FIGS. 2A-2C. Patterned layer 202 is overlaid on patterned layer 200 in a predetermined alignment. In this example, the predetermined alignment is such that channels 204 in patterned layer 200 and 202 are substantially perpendicular to each other. In some embodiments, as depicted in FIGS. 2A-2D, patterned layer 200 is overlaid on nonpatterned layer 206. Nonpatterned layer 206 can comprise a perfluoropolyether.

Continuing with reference to FIGS. 2A-2D, patterned layers 200 and 202, and in some embodiments nonpatterned layer 206, are treated by a treating process Tᵣ. As described in more detail herein below, layers 200, 202, and, in some embodiments nonpatterned layer 206, are treated by treating Tᵣ, to promote the adhesion of patterned layers 200 and 202 to each other, and in some embodiments, patterned layer 200 to nonpatterned layer 206, as shown in FIGS. 2C and 2D. The resulting microfluidic device 208 comprises an integrated network 210 of microscale channels 204 which intersect predetermined intersecting points 212, as best seen in the cross-section provided in FIG. 2D. Also shown in FIG. 2D is membrane 214 comprising the top surface of channels 204 of patterned layer 200 which separates channel 204 of patterned layer 202 from channels 204 of patterned layer 200.

Continuing with reference to FIGS. 2A-2C, in some embodiments, patterned layer 202 comprises a plurality of apertures, and the apertures are designated input aperture 216 and output aperture 218. In some embodiments, the holes, e.g., input aperture 216 and output aperture 218 are in fluid communication with channels 204. In some embodiments, the apertures comprise a side-actuated valve structure comprising a thin membrane of PFPE material which can be actuated to restrict the flow in an abutting channel (not shown).

In some embodiments, the first patterned layer of photocured PFPE material is cast at such a thickness to impart a degree of mechanical stability to the PFPE structure. Accordingly, in some embodiments, the first patterned layer of the photocured PFPE material is about 50 μm to several centimeters thick. In some embodiments, the first patterned layer of the photocured PFPE material is between 50 μm and about 10 millimeters thick. In some embodiments, the first patterned layer of the photocured PFPE material is 5 mm thick. In some embodiments, the first patterned layer of PFPE material is about 4 mm thick. Further, in some embodiments, the thickness of the first patterned layer of PFPE material ranges from about 0.1 μm to about 10 cm; from about 1 μm to about 5 cm; from about 10 μm to about 2 cm; and from about 100 μm to about 10 mm.

In some embodiments, the second patterned layer of the photocured PFPE material is between about 1 micrometer and about 100 micrometers thick. In some embodiments, the second patterned layer of the photocured PFPE material is between about 1 micrometer and about 50 micrometers thick. In some embodiments, the second patterned layer of the photocured material is about 20 micrometers thick.

Although FIGS. 2A-2C disclose the formation of a microfluidic device wherein two patterned layers of PFPE material are combined, in some embodiments of the presently disclosed subject matter it is possible to form a microfluidic device comprising one patterned layer and one non-patterned layer of PFPE material. Thus, the first patterned layer can comprise a microscale channel or an integrated network of microscale channels and then the first patterned layer can be overlaid on top of the non-patterned layer and adhered to the non-patterned layer using a photocuring step, such as application of ultraviolet light as disclosed herein, to form a monolithic structure comprising enclosed channels therein.

Accordingly, in some embodiments, a first and a second patterned layer of photocured perfluoropolyether material, or alternatively a first patterned layer of photocured perfluoropolyether material and a nonpatterned layer of photocured perfluoropolyether material, adhere to one another, thereby forming a monolithic PFPE-based microfluidic device.

III.C. Method of Forming a Patterned PFPE Layer Through a Thermal Free Radical Curing Process

In some embodiments, a thermal free radical initiator, including, but not limited to, a peroxide and/or an azo compound, is blended with a liquid perfluoropolyether (PFPE) precursor, which is functionalized with a polymerizable group, including, but not limited to, an acrylate, a methacrylate, and a styrenic unit to form a blend. As shown in FIGS. 1A-1C, the blend is then contacted with a patterned substrate, i.e., a “master,” and heated to cure the PFPE precursor into a network.

In some embodiments, the PFPE precursor is fully cured to form a fully cured PFPE precursor. In some embodiments, the free radical curing reaction is allowed to proceed only partially to form a partially-cured network.

III.D. Method of Adhering a Layer of a PFPE Material to a Substrate Through a Thermal Free Radical Curing Process

In some embodiments the fully cured PFPE precursor is removed, e.g., peeled, from the patterned substrate, i.e., the master, and contacted with a second substrate to form a reversible, hermetic seal.

In some embodiments, the partially cured network is contacted with a second partially cured layer of PFPE material and the curing reaction is taken to completion, thereby forming a permanent bond between the PFPE layers.

In some embodiments, the partial free-radical curing method is used to bond at least one layer of a partially-cured PFPE material to a substrate. In some embodiments, the partial free-radical curing method is used to bond a plurality of layers of a partially-cured PFPE material to a substrate. In some embodiments, the substrate is selected from the group consisting of a glass material, a quartz material, a silicon material, a fused silica material, and a plastic material. In some embodiments, the substrate is treated with a silane coupling agent.

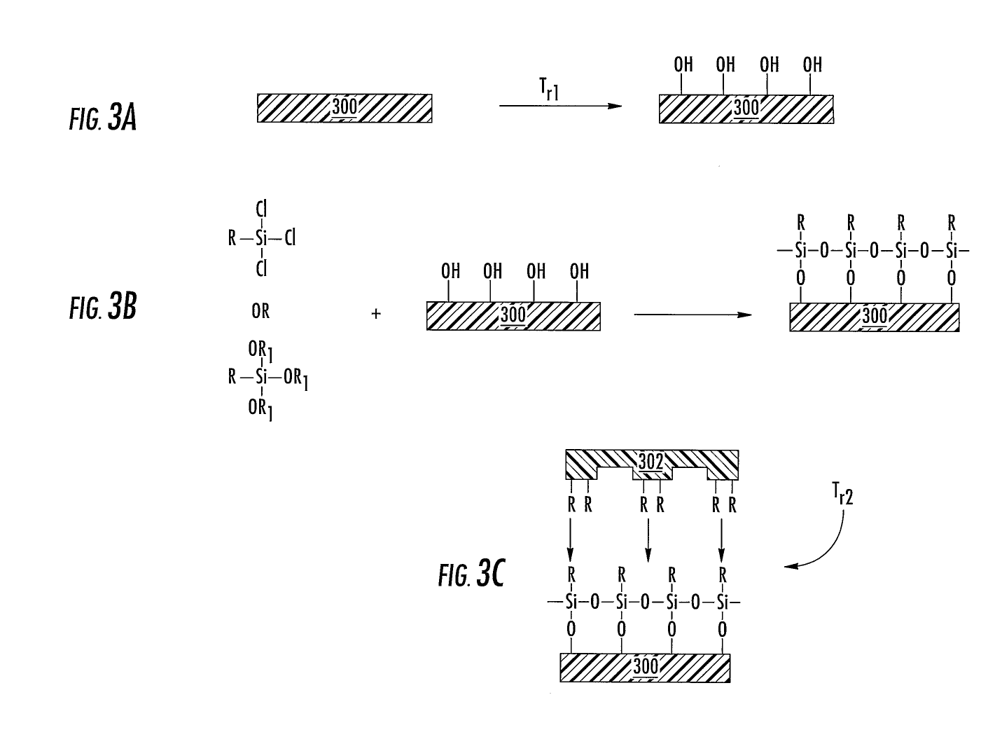

An embodiment of the presently disclosed method for adhering a layer of PFPE material to a substrate is illustrated in FIGS. 3A-3C. Referring now to FIG. 3A, a substrate 300 is provided, wherein, in some embodiments, substrate 300 is selected from the group consisting of a glass material, a quartz material, a silicon material, a fused silica material, and a plastic material. Substrate 300 is treated by treating process Tᵣ₁. In some embodiments, treating process Tᵣ₁ comprises treating the substrate with a base/alcohol mixture, e.g., KOH/isopropanol, to impart a hydroxyl functionality to substrate 300.

Referring now to FIG. 3B, functionalized substrate 300 is reacted with a silane coupling agent, e.g., R-SiCl₃ or R-Si(OR₁)₃, wherein R and R₁ represent a functional group as described herein to form a silanized substrate 300. In some embodiments, the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

Referring now to FIG. 3C, silanized substrate 300 is contacted with a patterned layer of partially cured PFPE material 302 and treated by treating process Tr₂ to form a permanent bond between patterned layer of PFPE material 302 and substrate 300.

In some embodiments, a partial free radical cure is used to adhere a PFPE layer to a second polymeric material, such as a poly(dimethyl siloxane) (PDMS) material, a polyurethane material, a silicone-containing polyurethane material, and a PFPE-PDMS block copolymer material. In some embodiments, the second polymeric material comprises a functionalized polymeric material. In some embodiments, the second polymeric material is encapped with a polymerizable group. In some embodiments, the polymerizable group is selected from the group consisting of an acrylate, a styrene, and a methacrylate. Further, in some embodiments, the second polymeric material is treated with a plasma and a silane coupling agent to introduce the desired functionality to the second polymeric material.

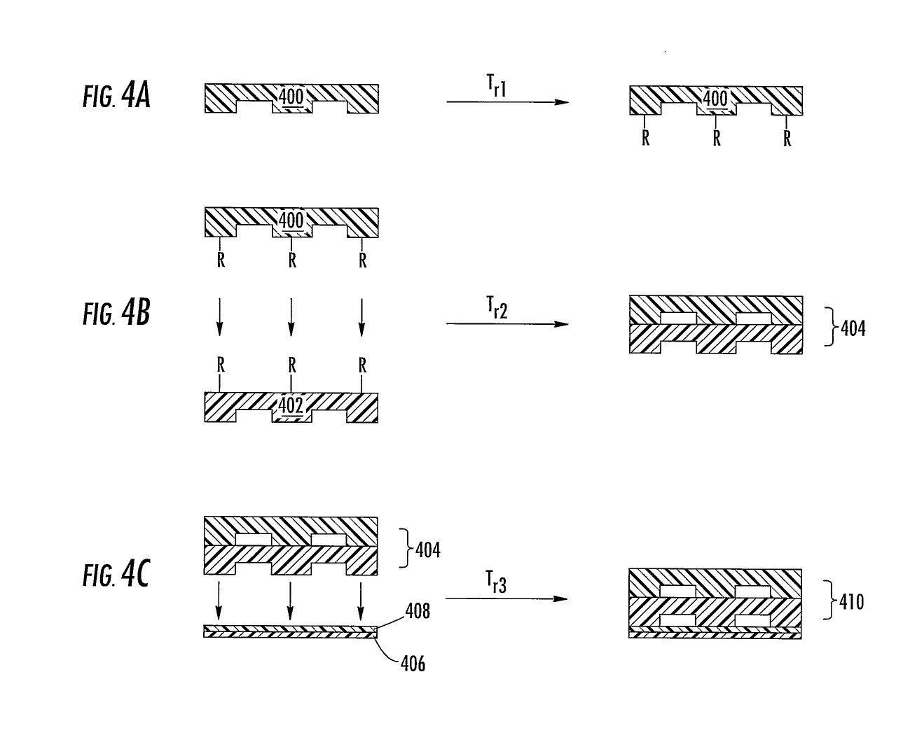

An embodiment of the presently disclosed method for adhering a patterned layer of PFPE material to another patterned layer of polymeric material is illustrated in FIGS. 4A-4C. Referring now to FIG. 4A, a patterned layer of a first polymeric material 400 is provided. In some embodiments, first polymeric material comprises a PFPE material. In some embodiments, first polymeric material comprises a polymeric material selected from the group consisting of a poly(dimethylsiloxane) material, a polyurethane material, a silicone-containing polyurethane material, and a PFPE-PDMS block copolymer material. Patterned layer of first polymeric material 400 is treated by treating process Tᵣ₁. In some embodiments, treating process Tᵣ₁ comprises exposing the patterned layer of first polymeric material 400 to UV light in the presence of O₃ and an R functional group, to add an R functional group to the patterned layer of polymeric material 400.

Referring now to FIG. 4B, the functionalized patterned layer of first polymeric material 400 is contacted with the top surface of a functionalized patterned layer of PFPE material 402 and then treated by treating process Tᵣ₂ to form a two layer hybrid assembly 404. Thus, functionalized patterned layer of first polymeric material 400 is thereby bonded to functionalized patterned layer of PFPE material 402.

Referring now to FIG. 4C, two-layer hybrid assembly 404, in some embodiments, is contacted with substrate 406 to form a multilayer hybrid structure 410. In some embodiments, substrate 406 is coated with a layer of liquid PFPE precursor material 408. Multilayer hybrid structure 410 is treated by treating process Tᵣ₃ to bond two-layer assembly 404 to substrate 406.

IV. Method for Forming a Microfluidic Device Through a Two-Component Curing Process

The presently disclosed subject matter provides a method for forming a microfluidic device by which functional perfluoropolyether (PFPE) precursors are contacted with a patterned surface and then cured through the reaction of two components, such as epoxy/amine, hydroxyl/isocyanate, hydroxyl/acid chloride, and hydroxyl/chlorosilane, to form a fully-cured or a partially-cured PFPE network. In some embodiments, the partially-cured PFPE network is contacted with another substrate, and the curing is then take to completion to adhere the PFPE network to the substrate. This method can be used to adhere multiple layers of a PFPE material to a substrate.

Further, in some embodiments, the substrate comprises a second polymeric material, such as PDMS, or another polymer. In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic material, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, the PFPE layer is adhered to a solid substrate, such as a glass material, a quartz material, a silicon material, and a fused silica material, through use of a silane coupling agent.

IV.A. Method of Forming a Patterned PFPE Layer Through a Two-Component Curing Process

In some embodiments, a PFPE network is formed through the reaction of a two-component functional liquid precursor system. Using the general method for forming a patterned layer of polymeric material as shown in FIGS. 1A-1C, a liquid precursor material comprising a two-component system is contacted with a patterned substrate and a patterned layer of PFPE material is formed. In some embodiments, the two-component liquid precursor system is selected from the group consisting of an epoxy/amine system, a hydroxyl/isocyanate system, an amine/isocyanate system, a hydroxyl/acid chloride system, and a hydroxyl/chlorosilane system. The functional liquid precursors are blended in the appropriate ratios and then contacted with a patterned surface or master. The curing reaction is allowed to take place by using heat, catalysts, and the like, until the network is formed.

In some embodiments, a fully cured PFPE precursor is formed. In some embodiments, the two-component reaction is allowed to proceed only partially, thereby forming a partially cured PFPE network.

IV. B. Method of Adhering a PFPE Layer to a Substrate Through a Two-Component Curing Process

IV.B.1. Full Cure with a Two-Component Curing Process

In some embodiments, the fully cured PFPE two-component precursor is removed, e.g., peeled, from the master and contacted with a substrate to form a reversible, hermetic seal. In some embodiments, the partially cured network is contacted with another partially cured layer of PFPE and the reaction is taken to completion, thereby forming a permanent bond between the layers.

IV.B.2. Partial Cure with a Two-Component System

As shown in FIGS. 3A-3C, in some embodiments, the partial two-component curing method is used to bond at least one layer of a partially-cured PFPE material to a substrate. In some embodiments, the partial two-component curing method is used to bond a plurality of layers of a partially-cured PFPE material to a substrate. In some embodiments, the substrate is selected from the group consisting of a glass material, a quartz material, a silicon material, a fused silica material, and a plastic material. In some embodiments, the substrate is treated with a silane coupling agent.

As shown in FIGS. 4A-4C, in some embodiments, a partial two-component cure is used to adhere the PFPE layer to a second polymeric material, such as a poly(dimethylsiloxane) (PDMS) material. In some embodiments, the PDMS material comprises a functionalized PDMS material. In some embodiments, the PDMS is treated with a plasma and a silane coupling agent to introduce the desired functionality to the PDMS material. In some embodiments, the PDMS material is encapped with a polymerizable group. In some embodiments, the polymerizable group comprises an epoxide. In some embodiments, the polymerizable group comprises an amine.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

IV.B.3. Excess Cure with a Two-Component System

The presently disclosed subject matter provides a method for forming a microfluidic device by which a functional perfluoropolyether (PFPE) precursor is contacted with a patterned substrate and cured through the reaction of two components, such as epoxy/amine, hydroxyl/isocyanate, hydroxyl/acid chloride, and hydroxyl/chlorosilane, to form a layer of cured PFPE material. In this particular method, the layer of cured PFPE material can be adhered to a second substrate by fully curing the layer with an excess of one component and contacting the layer of cured PFPE material with a second substrate comprising an excess of a second component in such a way that the excess groups react to adhere the layers.

Thus, in some embodiments, a two-component system, such as an epoxy/amine system, a hydroxyl/isocyanate system, an amine/isocyanate system, a hydroxyl/acid chloride system, or a hydroxyl/chlorosilane system, is blended. In some embodiments, at least one component of the two-component system is in excess of the other component. The reaction is then taken to completion by heating, using a catalyst, and the like, with the remaining cured network comprising a plurality of functional groups generated by the presence of the excess component.

In some embodiments, two layers of fully cured PFPE materials comprising complimentary excess groups are contacted with one another, wherein the excess groups are allowed to react, thereby forming a permanent bond between the layers.

As shown in FIGS. 3A-3C, in some embodiments, a fully cured PFPE network comprising excess functional groups is contacted with a substrate. In some embodiments, the substrate is selected from the group consisting of a glass material, a quartz material, a silicon material, a fused silica material, and a plastic material. In some embodiments, the substrate is treated with a silane coupling agent such that the functionality on the coupling agent is complimentary to the excess functionality on the fully cured network. Thus, a permanent bond is formed to the substrate.

As shown in FIGS. 4A-4C, in some embodiments, the two-component excess cure is used to bond a PFPE network to a second polymeric material, such as a poly(dimethylsiloxane) PDMS material. In some embodiments, the PDMS material comprises a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable material comprises an epoxide. In some embodiments, the polymerizable material comprises an amine.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

V. Method for Functionalizing a Surface of a Micro- and/or Nano-Scale Device

In some embodiments, the presently disclosed subject matter provides materials and methods for functionalizing the channels in a microfluidic device and/or a microtiter well. In some embodiments, such functionalization includes, but is not limited to, the synthesis and/or attachment of peptides and other natural polymers to the interior surface of a channel in a microfluidic device. Accordingly, the presently disclosed subject matter can be applied to microfluidic devices, such as those described by Rolland, J., et al., JACS 2004, 126, 2322-2323, the disclosure of which is incorporated herein by reference in its entirety.

In some embodiments, the method comprises binding a small molecule to the interior surface of a microfluidic channel or the surface of a microtiter well. In such embodiments, once bound, the small molecule can serve a variety of functions. In some embodiments, the small molecule functions as a cleavable group, which when activated, can change the polarity of the channel and hence the wettability of the channel. In some embodiments, the small molecule functions as a binding site. In some embodiments, the small molecule functions as a binding site for one of a catalyst, a drug, a substrate for a drug, an analyte, and a sensor. In some embodiments, the small molecule functions as a reactive functional group. In some embodiments, the reactive functional group is reacted to yield a Zwitterion. In some embodiments, the Zwitterion provides a polar, ionic channel.

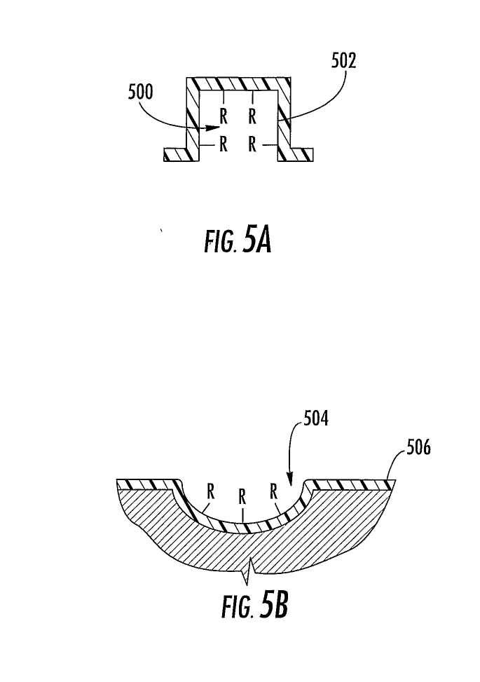

An embodiment of the presently disclosed method for functionalizing the interior surface of a microfluidic channel and/or a microtiter well is illustrated in FIGS. 5A and 5B. Referring now to FIG. 5A, a microfluidic channel 500 is provided. In some embodiments, microfluidic channel 500 is formed from a functional PFPE material comprising an R functional group, as described herein. In some embodiments, microchannel 500 comprises a PFPE network which undergoes a post-curing treating process, whereby functional group R is introduced into the interior surface 502 of microfluidic channel 500.

Referring now to FIG. 5B, a microtiter well 504 is provided. In some embodiments, microtiter well 504 is coated with a layer of functionalized PFPE material 506, which comprises an R functional group, to impart functionality into microtiter well 504.

V.A. Method of Attaching a Functional Group to a PFPE Network

In some embodiments, PFPE networks comprising excess functionality are used to functionalize the interior surface of a microfluidic channel or the surface of a microtiter well. In some embodiments, the interior surface of a microfluidic channel or the surface of a microtiter well is functionalized by attaching a functional moiety selected from the group consisting of a protein, an oligonucleotide, a drug, a ligand, a catalyst, a dye, a sensor, an analyte, and a charged species capable of changing the wettability of the channel.

In some embodiments, latent functionalities are introduced into the fully cured PFPE network. In some embodiments, latent methacrylate groups are present at the surface of the PFPE network that has been free radically cured either photochemically or thermally. Multiple layers of fully cured PFPE are then contacted with the functionalized surface of the PFPE network, forming a seal, and reacted, by heat, for example, to allow the latent functionalities to react and form a permanent bond between the layers.

In some embodiments, the latent functional groups react photochemically with one another at a wavelength different from that used to cured PFPE precursors. In some embodiments, this method is used to adhere fully cured layers to a substrate. In some embodiments, the substrate is selected from the group consisting of a glass material, a quartz material, a silicon material, a fused silica material, and a plastic material. In some embodiments, the substrate is treated with a silane coupling agent complimentary to the latent functional groups.

In some embodiments, such latent functionalities are used to adhere a fully cured PFPE network to a second polymeric material, such as a poly(dimethylsiloxane) PDMS material. In some embodiments, the PDMS material comprises a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable group is selected from the group consisting of an acrylate, a styrene, and a methacrylate.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

V.B. Method of Introducing Functionality in the Generation of a Liquid PFPE Precursor

The presently disclosed subject matter provides a method of forming a microfluidic device by which a photochemically cured PFPE layer is placed in conformal contact with a second substrate thereby forming a seal. The PFPE layer is then heated at elevated temperatures to adhere the layer to the substrate through latent functional groups. In some embodiments, the second substrate also comprises a cured PFPE layer. In some embodiments, the second substrate comprises a second polymeric material, such as a poly(dimethylsiloxane) (PDMS) material.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, the latent groups comprise methacrylate units that are not reacted during the photocuring process. Further, in some embodiments, the latent groups are introduced in the generation of the liquid PFPE precursor. For example, in some embodiments, methacrylate units are added to a PFPE diol through the use of glycidyl methacrylate, the reaction of the hydroxy and the epoxy group generates a secondary alcohol, which can be used as a handle to introduce chemical functionality. In some embodiments, multiple layers of fully cured PFPE are adhered to one another through these latent functional groups. In some embodiments, the latent functionalities are used to adhere a fully cured PFPE layer to a substrate. In some embodiments, the substrate is selected from the group consisting of a glass material, a quartz material, a silicon material, a fused silica material, and a plastic material. In some embodiments, the substrate is treated with a silane coupling agent.

Further, this method can be used to adhere a fully cured PFPE layer to a second polymeric material, such as a poly(dimethylsiloxane) (PDMS) material. In some embodiments, the PDMS material comprises a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable material is selected from the group consisting of an acrylate, a styrene, and a methacrylate.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, PFPE networks containing latent functionality are used to functionalize the interior surface of a microfluidic channel or a microtiter well. Examples include the attachment of proteins, oligonucleotides, drugs, ligands, catalysts, dyes, sensors, analytes, and charged species capable of changing the wettability of the channel.

V.C. Method of Linking Multiple Chains of a PFPE Material with a Functional Linker Group

In some embodiments, the presently disclosed method adds functionality to a microfluidic channel or a microtiter well by adding a chemical “linker” moiety to the elastomer itself. In some embodiments, a functional group is added along the backbone of the precursor material. An example of this method is illustrated in Scheme 6.

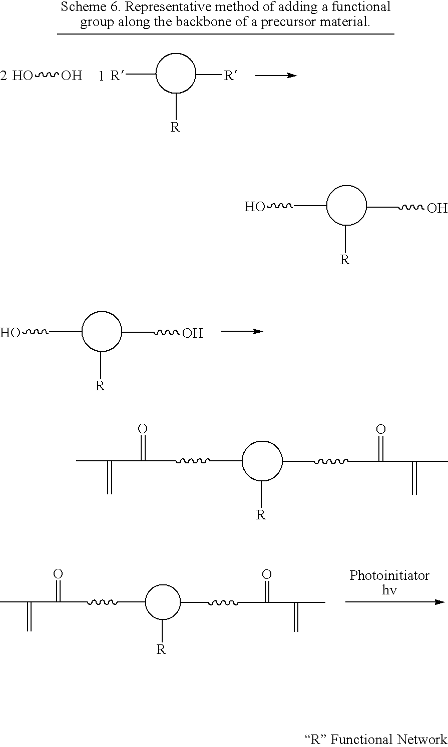

In some embodiments, the precursor material comprises a macromolecule containing hydroxyl functional groups. In some embodiments, as depicted in Scheme 6, the hydroxyl functional groups comprise diol functional groups. In some embodiments, two or more of the diol functional groups are connected through a trifunctional “linker” molecule. In some embodiments, the trifunctional linker molecule has two functional groups, R and R′. In some embodiments, the R′ group reacts with the hydroxyl groups of the macromolecule. In Scheme 6, the circle can represent a linking molecule; and the wavy line can represent a PFPE chain.

In some embodiments, the R group provides the desired functionality to the interior surface of the microfluidic channel or surface of a microtiter well. In some embodiments, the R′ group is selected from the group including, but not limited to, an acid chloride, an isocyanate, a halogen, and an ester moiety. In some embodiments, the R group is selected from one of, but not limited to, a protected amine and a protected alcohol. In some embodiments, the macromolecule diol is functionalized with polymerizable methacrylate groups. In some embodiments, the functionalized macromolecule diol is cured and/or molded by a photochemical process as described by Rolland, J. et al. JACS 2004, 126, 2322-2323, the disclosure of which is incorporated herein by reference in its entirety.

Thus, the presently disclosed subject matter provides a method of incorporating latent functional groups into a photocurable PFPE material through a functional linker group. Thus, in some embodiments, multiple chains of a PFPE material are linked together before encapping the chain with a polymerizable group. In some embodiments, the polymerizable group is selected from the group consisting of a methacrylate, an acrylate, and a styrenic. In some embodiments, latent functionalities are attached chemically to such “linker” molecules in such a way that they will be present in the fully cured network.

In some embodiments, latent functionalities introduced in this manner are used to bond multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material is treated with a plasma and a silane coupling agent to introduce the desired functionality. In some embodiments, the PDMS material is encapped with a polymerizable group. In some embodiments, the polymerizable group is selected from the group consisting of an acrylate, a styrene, and a methacrylate.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, PFPE networks comprising functionality attached to “linker” molecules are used to functionalize the interior surface of a microfluidic channel and/or the surface of a microtiter well. In some embodiments, the inside of a microfluidic channel is functionalized by attaching a functional moiety selected from the group consisting of a protein, an oligonucleotide, a drug, a catalyst, a dye, a sensor, an analyte, and a charged species capable of changing the wettability of the channel.

VI. Method of Adding Functional Monomers to the PFPE Precursor Material

In some embodiments, the method comprises adding a functional monomer to an uncured precursor material. In some embodiments, the functional monomer is selected from the group consisting of functional styrenes, methacrylates, and acrylates. In some embodiments, the precursor material comprises a fluoropolymer. In some embodiments, the functional monomer comprises a highly fluorinated monomer. In some embodiments, the highly fluorinated monomer comprises perfluoro ethyl vinyl ether (EVE). In some embodiments, the precursor material comprises a poly(dimethyl siloxane) (PDMS) elastomer. In some embodiments, the precursor material comprises a polyurethane elastomer. In some embodiments, the method further comprises incorporating the functional monomer into the network by a curing step.

In some embodiments, functional monomers are added directly to the liquid PFPE precursor to be incorporated into the network upon crosslinking. For example, monomers can be introduced into the network that are capable of reacting post-crosslinking to adhere multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material is treated with a plasma and a silane coupling agent to introduce the desired functionality. In some embodiment, the PDMS material is encapped with a polymerizable group. In some embodiments, the polymerizable material is selected from the group consisting of an acrylate, a styrene, and a methacrylate.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, functional monomers are added directly to the liquid PFPE precursor and are used to attach a functional moiety selected from the group consisting of a protein, an oligonucleotide, a drug, a catalyst, a dye, a sensor, an analyte, and a charged species capable of changing the wettability of the channel.

Such monomers include, but are not limited to, tert-butyl methacrylate, tert butyl acrylate, dimethylaminopropyl methacrylate, glycidyl methacrylate, hydroxy ethyl methacrylate, aminopropyl methacrylate, allyl acrylate, cyano acrylates, cyano methacrylates, trimethoxysilane acrylates, trimethoxysilane methacrylates, isocyanato methacrylate, lactone-containing acrylates and methacrylates, sugar-containing acrylates and methacrylates, poly-ethylene glycol methacrylate, nornornane-containing methacrylates and acrylates, polyhedral oligomeric silsesquioxane methacrylate, 2-trimethylsiloxyethyl methacrylate, 1H,1H,2H,2H-fluoroctylmethacrylate, pentafluorostyrene, vinyl pyridine, bromostyrene, chlorostyrene, styrene sulfonic acid, fluorostyrene, styrene acetate, acrylamide, and acrylonitrile.

In some embodiments, monomers which already have the above agents attached are blended directly with the liquid PFPE precursor to be incorporated into the network upon crosslinking. In some embodiments, the monomer comprises a group selected from the group consisting of a polymerizable group, the desired agent, and a fluorinated segment to allow for miscibility with the PFPE liquid precursor. In some embodiments, the monomer does not comprise a polymerizable group, the desired agent, and a fluorinated segment to allow for miscibility with the PFPE liquid precursor.

In some embodiments, monomers are added to adjust the mechanical properties of the fully cured elastomer. Such monomers include, but are not limited to: perfluoro(2,2-dimethyl-1,3-dioxole), hydrogen-bonding monomers which contain hydroxyl, urethane, urea, or other such moieties, monomers containing bulky side group, such as tert-butyl methacrylate.

In some embodiments, functional species such as the above mentioned monomers are introduced and are mechanically entangled, i.e., not covalently bonded, into the network upon curing. For example, in some embodiments, functionalities are introduced to a PFPE chain that does not contain a polymerizable monomer and such a monomer is blended with the curable PFPE species. In some embodiments, such entangled species can be used to adhere multiple layers of cured PFPE together if two species are reactive, such as: epoxy/amine, hydroxy/acid chloride, hydroxy/isocyanate, amine/isocyanate, amine/halide, hydroxy/halide, amine/ester, and amine/carboxylic acid. Upon heating, the functional groups will react and adhere the two layers together.

Additionally, such entangled species can be used to adhere a PFPE layer to a layer of another material, such as glass, silicon, quartz, PDMS, Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, such an entangled species can be used to functionalize the interior of a microfluidic channel for the purposes described hereinabove.

VII. Other Methods of Introducing Functionality to a PFPE Surface

In some embodiments, an Argon plasma is used to introduce functionality along a fully cured PFPE surface using the method for functionalizing a poly(tetrafluoroethylene) surface as described by Chen, Y. and Momose, Y. Surf. Interface. Anal. 1999, 27, 1073-1083, which is incorporated herein by reference in it entirety. More particularly, without being bound to any one particular theory, exposure of a fully cured PFPE material to Argon plasma for a period of time adds functionality along the fluorinated backbone.

Such functionality can be used to adhere multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material comprises a functionalized material. In some embodiments, the PDMS material is treated with a plasma and a silane coupling agent to introduce the desired functionality. Such functionalities also can be used to attach proteins, oligonucleotides, drugs, catalysts, dyes, sensors, analytes, and charged species capable of changing the wettability of the channel.

In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, a fully cured PFPE layer is brought into conformal contact with a solid substrate. In some embodiments, the solid substrate is selected from the group consisting of a glass material, a quartz material, a silicon material, a fused silica material, and a plastic material. In some embodiments, the PFPE material is irradiated with UV light, e.g., a 185 nm UV light, which can strip a fluorine atom off of the back bone and form a chemical bond to the substrate as described by Vurens, G., et al. Langmuir 1992, 8, 1165-1169. Thus, in some embodiments, the PFPE layer is covalently bonded to the solid substrate by radical coupling following abstraction of a fluorine atom.

VIII. Adhesion of a Microscale or a Nanoscale Device to a Substrate Through an Encasing Polymer

In some embodiments, a microscale device, a nanoscale device, or combinations thereof is adhered to a substrate by placing the fully cured device in conformal contact on the substrate and pouring an “encasing polymer” over the entire device. In some embodiments, the encasing polymer is selected from the group consisting of a liquid epoxy precursor and a polyurethane. The encasing polymer is then solidified by curing or other methods. The encasement serves to bind the layers together mechanically and to bind the layers to the substrate.

In some embodiments, the microscale device, the nanoscale device, or combinations thereof comprises one of a perfluoropolyether material as described in Section II.A and Section II.B. hereinabove and a fluoroolefin-based material as described in Section II.C. hereinabove.

In some embodiments, the substrate is selected from the group consisting of a glass material, a quartz material, a silicon material, a fused silica material, and a plastic material. Further, in some embodiments, the substrate comprises a second polymeric material, such as poly(dimethylsiloxane) (PDMS), or another polymer. In some embodiments, the second polymeric material comprises an elastomer other than PDMS, such as Kratons, buna rubber, natural rubber, a fluorelastomer, chloroprene, butyl rubber, nitrile rubber, polyurethane, or a thermoplastic elastomer. In some embodiments, the second polymeric material comprises a rigid thermoplastic material, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone). In some embodiments, the surface of the substrate is functionalized with a silane coupling agent such that it will react with the encasing polymer to form an irreversible bond.

IX. Method for Forming a Microstructure Using Sacrificial Layers

The presently disclosed subject matter provides a method for forming microchannels or a microstructure for use as a microfluidic device by using sacrificial layers comprising a degradable or selectively soluble material. In some embodiments, the method comprises contacting a liquid precursor material with a two-dimensional or a three-dimensional sacrificial structure, treating, e.g., curing, the precursor material, and removing the sacrificial structure to form a microfluidic channel.

Accordingly, in some embodiments, a PFPE liquid precursor is disposed on a multidimensional scaffold, wherein the multidimensional scaffold is fabricated from a material that can be degraded or washed away after curing of the PFPE network. These materials protect the channels from being filled in when another layer of elastomer is cast thereon. Examples of such degradable or selective soluble materials include, but are not limited to waxes, photoresists, polysulfones, polylactones, cellulose fibers, salts, or any solid organic or inorganic compounds. In some embodiments, the sacrificial layer is removed thermally, photochemically, or by washing with solvents. Importantly, the compatibility of the materials and devices disclosed herein with organic solvents provides the capability to use sacrificial polymer structures in microfluidic devices.

The PFPE materials of use in forming a microstructure by using sacrificial layers include those PFPE and fluoroolefin-based materials as described hereinabove in Section II of the presently disclosed subject matter.

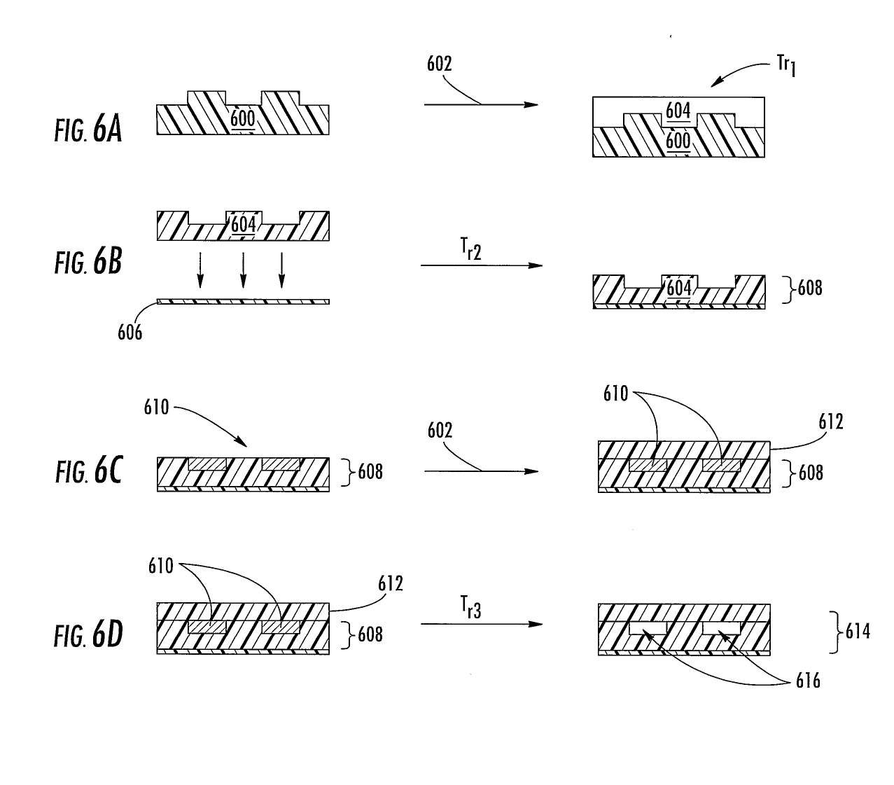

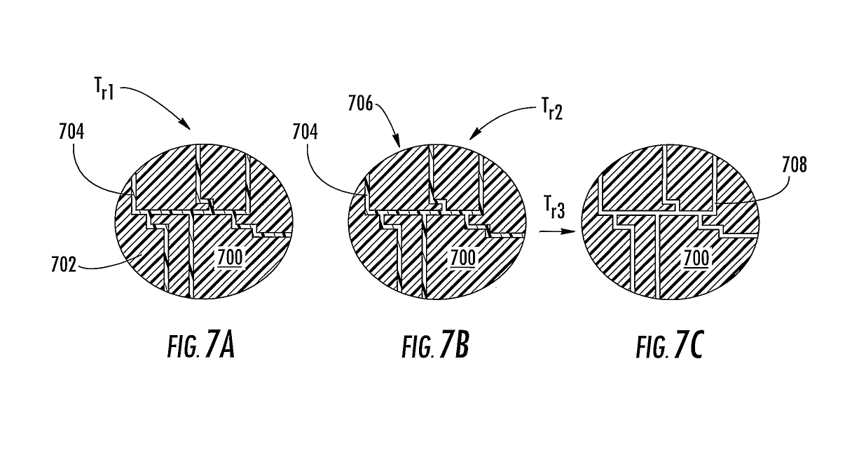

FIGS. 6A-6D and FIGS. 7A-7C show embodiments of the presently disclosed methods for forming a microstructure by using a sacrificial layer of a degradable or selectively soluble material.

Referring now to FIG. 6A, a patterned substrate 600 is provided. Liquid PFPE precursor material 602 is disposed on patterned substrate 600. In some embodiments, liquid PFPE precursor material 602 is disposed on patterned substrate 600 via a spin-coating process. Liquid PFPE precursor material 602 is treated by treating process Tᵣ₁ to form a layer of treated liquid PFPE precursor material 604.

Referring now to FIG. 6B, the layer of treated liquid PFPE precursor material 604 is removed from patterned substrate 600. In some embodiments, the layer of treated liquid PFPE precursor material 604 is contacted with substrate 606. In some embodiments, substrate 606 comprises a planar substrate or a substantially planar substrate. In some embodiments, the layer of treated liquid PFPE precursor material is treated by treating process Tᵣ₂, to form two-layer assembly 608.

Referring now to FIG. 6C, a predetermined volume of degradable or selectively soluble material 610 is disposed on two-layer assembly 608. In some embodiments, the predetermined volume of degradable or selectively soluble material 610 is disposed on two-layer assembly 608 via a spin-coating process. Referring once again to FIG. 6C, liquid precursor material 602 is disposed on two-layer assembly 608 and treated to form a layer of PFPE material 612, which covers the predetermined volume of degradable or selectively soluble material 610.

Referring now to FIG. 6D, the predetermined volume of degradable or selectively soluble material 610 is treated by treating process Tᵣ₃ to remove the predetermined volume of degradable or selectively soluble material 610, thereby forming microstructure 616. In some embodiments, microstructure 616 comprises a microfluidic channel. In some embodiments, treating process Tᵣ₃ is selected from the group consisting of a thermal process, an irradiation process, and a dissolution process.

In some embodiments, patterned substrate 600 comprises an etched silicon wafer. In some embodiments, the patterned substrate comprises a photoresist patterned substrate. For the purposes of the presently disclosed subject matter, the patterned substrate can be fabricated by any of the processing methods known in the art, including, but not limited to, photolithography, electron beam lithography, and ion milling.

In some embodiments, degradable or selectively soluble material 610 is selected from the group consisting of a polyolefin sulfone, a cellulose fiber, a polylactone, and a polyelectrolyte. In some embodiments, the degradable or selectively soluble material 610 is selected from a material that can be degraded or dissolved away. In some embodiments, degradable or selectively soluble material 610 is selected from the group consisting of a salt, a water-soluble polymer, and a solvent-soluble polymer.

In addition to simple channels, the presently disclosed subject matter also provides for the fabrication of multiple complex structures that can be “injection molded” or fabricated ahead of time and embedded into the material and removed as described above.

FIGS. 7A-C illustrate an embodiment of the presently disclosed method for forming a microchannel or a microstructure through the use of a sacrificial layer. Referring now to FIG. 7A, a substrate 700 is provided. In some embodiments, substrate 700 is coated with a liquid PFPE precursor material 702. Sacrificial structure 704 is placed on substrate 700. In some embodiments, liquid PFPE precursor material 702 is treated by treating process Tᵣ₁.

Referring now to FIG. 7B, a second liquid PFPE precursor material 706 is disposed over sacrificial structure 704, in such a way to encase sacrificial structure 704 in second liquid precursor material 706. Second liquid precursor material 706 is then treated by treating process Tᵣ₂. Referring now to FIG. 7C, sacrificial structure 704 is treated by treating process Tᵣ₃, to degrade and/or remove sacrificial structure, thereby forming microstructure 708. In some embodiments, microstructure 708 comprises a microfluidic channel.

In some embodiments, substrate 700 comprises a silicon wafer. In some embodiments, sacrificial structure 704 comprises a degradable or selectively soluble material. In some embodiments, sacrificial structure 704 is selected from the group consisting of a polyolefin sulfone, a cellulose fiber, a polylactone, and a polyelectrolyte. In some embodiments, the sacrificial structure 704 is selected from a material that can be degraded or dissolved away. In some embodiments, sacrificial structure 704 is selected from the group consisting of a salt, a water-soluble polymer, and a solvent-soluble polymer.

X. Microfluidics Unit Operations

Microfluidic control devices are necessary for the development of effective lab-on-a-chip operations. Valve structures and actuation, fluid control, mixing, separation, and detection at microscale levels must be designed to have a large-scale shift to miniaturization. To construct such devices, integration of the individual components on a common platform must be developed so that solvents and solutes can be completely controlled.

Microfluidic flow controllers are traditionally externally pump-based, including hydrodynamic, reciprocating, acoustic, and peristaltic pumps, and can be as simple as a syringe (see U.S. Pat. No. 6,444,106 to Mcbride et al., U.S. Pat. No. 6,811,385 to Blakley, U.S. Published Patent Application No. 20040028566 to Ko et al.). More recently, electroosmosis, a process that does not require moving parts, has experienced success as a fluid flow driver (see U.S. Pat. No. 6,406,605 to Moles, U.S. Pat. No. 6,568,910 to Parse). Other fluid flow devices that do not require moving parts use gravity (see U.S. Pat. No. 6,743,399 to Weigl et al.), centrifugal force (see U.S. Pat. No. 6,632,388 to Sanae et al.), capillary action (see U.S. Pat. No. 6,591,852 to McNeely et al.), or heat (see U.S. Published Patent Application No. 20040257668 to Ito) to drive liquids through the microchannels. Other inventions create liquid flow by the application of an external force, such as a blade (see U.S. Pat. No. 6,068,751 to Neukermans).

Valves also are used in fluid flow control. Valves can be actuated by applying an external force, such as a blade, cantilever, or plug to an elastomeric channel (see U.S. Pat. No. 6,068,751 to Neukermans). Elastic channels also can contain membranes that can be deflected by air pressure and/or liquid pressure, e.g., water pressure, electrostatically, or magnetically (see U.S. Pat. No. 6,408,878 to Unger et al.). Other 2-way valves are actuated by light (see U.S. Published Patent Application No. 20030156991 to Halas et al.), piezoelectric crystals (see Published PCT International Application No. WO 2003/089,138 to Davis et al.), particle deflection (see U.S. Pat. No. 6,802,489 to Marr et al.), or bubbles formed within the channel electrochemically (see Published PCT International Application No. WO 2003/046,256 to Hua et al.). One-way or “check valves” also can be formed in microchannels with balls, flaps, or diaphragms (see U.S. Pat. No. 6,817,373 to Cox et al.; U.S. Pat. No. 6,554,591 to Dai et al.; Published PCT International Application No. WO 2002/053,290 to Jeon et al.). Rotary-type switching valves are used for complex reactions (see Published PCT International Application No. WO 2002/055,188 to Powell et al.).

Microscale mixing and separation components are necessary to facilitate reactions and evaluate products. In microfluidic devices, mixing is most often done by diffusion, in channels of long length scales, curved, with variable widths, or having features that cause turbulence (see U.S. Pat. No. 6,729,352 to O'Conner et al., U.S. Published Patent Application No. 20030096310 to Hansen et al.). Mixing also can be accomplished electroosmotically (see U.S. Pat. No. 6,482,306 to Yager et al.) or ultrasonically (see U.S. Pat. No. 5,639,423 to Northrup et al.). Separations in micro-scale channels typically use three methods: electrophoresis, packed columns or gel within a channel, or functionalization of channel walls. Electrophoresis is commonly done with charged molecules, such as nucleic acids, peptides, proteins, enzymes, and antibodies and the like, and is the simplest technique (see U.S. Pat. No. 5,958,202 to Regnier et al., U.S. Pat. No. 6,274,089 to Chow et al.). Channel columns can be packed with porous or stationary-phase coated beads or a gel to facilitate separations (see Published PCT International Application No. WO 2003/068,402 to Koehler et al., U.S. Published Patent Application No. 20020164816 to Quake et al., U.S. Pat. No. 6,814,859 to Koehler et al.). Possible packing materials include silicates, talc, Fuller's earth, glass wool, charcoal, activated charcoal, celite, silica gel, alumina, paper, cellulose, starch, magnesium silicate, calcium sulfate, silicic acid, florisil, magnesium oxide, polystyrene, p-aminobenzyl cellulose, polytetrafluoroethylene resin, polystyrene resin, SEPHADEX™ (Amersham Biosciences, Corp., Piscataway, N.J., United States of America), SEPHAROSE™ (Amersham Biosciences, Corp., Piscataway, N.J., United States of America), controlled pore glass beads, agarose, other solid resins known to one skilled in the art and combinations of two or more of any of the foregoing. Magnetizable material, such as ferric oxide, nickel oxide, barium ferrite or ferrous oxide, also can be imbedded, encapsulated of otherwise incorporated into a solid-phase packing material.

The walls of microfluidic chambers also can be functionalized with a variety of ligands that can interact or bind to an analyte or to a contaminant in an analyte solution. Such ligands include: hydrophilic or hydrophobic small molecules, steroids, hormones, fatty acids, polymers, RNA, DNA, PNA, amino acids, peptides, proteins (including antibody binding proteins such as protein G), antibodies or antibody fragments (FABs, etc), antigens, enzymes, carbohydrates (including glycoproteins or glycolipids), lectins, cell surface receptors (or portions thereof), species containing a positive or a negative charge, and the like (see U.S. Published Patent Application No. 20040053237 to Liu et al., Published PCT International Application No. WO 2004/007,582 to Augustine et al., U.S. Published Patent Application No. 20030190608 to Blackburn).

Thus, in some embodiments, the presently disclosed subject matter describes a method of flowing a material and/or mixing two or more materials in a PFPE-based microfluidic device. In some embodiments, the presently disclosed subject matter describes a method of conducting a chemical reaction, including but not limited to synthesizing a biopolymer, such as DNA. In some embodiments, the presently disclosed subject matter describes a method of screening a sample for a characteristic. In some embodiments, the presently disclosed subject matter describes a method of dispensing a material. In some embodiments, the presently disclosed subject matter describes a method of separating a material.

X.A. Method of Flowing a Material and/or Mixing Two Materials in a PFPE-Based Microfluidic Device

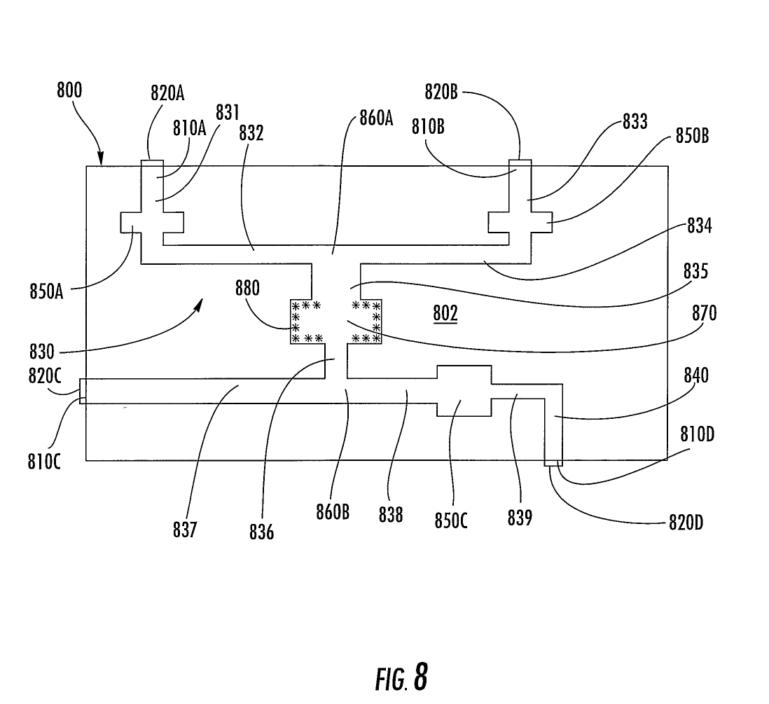

Referring now to FIG. 8, a schematic plan view of a microfluidic device of the presently disclosed subject matter is shown. The microfluidic device is referred to generally at 800. Microfluidic device 800 comprises a patterned layer 802, and a plurality of holes 810A, 810B, 810C, and 810D. These holes can be further described as inlet aperture 810A, inlet aperture 810B, and inlet aperture 810C, and outlet aperture 810D. Each of apertures 810A, 810B, 810C, and 810D are covered by seals 820A, 820B, 820C, and 820D, which are preferably reversible seals. Seals 820A, 820B, 820C, and 820D are provided so that materials, including but not limited to, solvents, chemical reagents, components of a biochemical system, samples, inks, and reaction products and/or mixtures of solvents, chemical reagents, components of a biochemical system, samples, inks, reaction products and combinations thereof, can be stored, shipped, or otherwise maintained in microfluidic device 800 if desired. Seals 820A, 820B, 820C, and 820D can be reversible, that is, removable, so that microfluidic device 800 can be implemented in a chemical reaction or other use and then can be resealed if desired.

Continuing with reference to FIG. 8, in some embodiments, apertures 810A, 810B, and 810C, further comprise pressure-actuated valves (comprising intersecting, overlaid flow channels not shown) which can be actuated to seal the microfluidic channel associated with the aperture.

Continuing with reference to FIG. 8, patterned layer 802 of microfluidic device 800 comprises an integrated network 830 of microscale channels. Optionally, pattern layer 802 comprises a functionalized surface, such as that shown in FIG. 5A. Integrated network 830 can comprise a series of fluidly connected microscale channels designated by the following reference characters: 831, 832, 833, 834, 835, 836, 837, 838, 839, and 840. Thus, inlet aperture 810A is in fluid communication with microscale channel 831 that extends away from aperture 810A and is in fluid communication with microscale channel 832 via a bend. In integrated network 830 depicted in FIG. 8, a series of 90° bends are shown for convenience. It is noted, however, that the paths and bends provided in the channels of integrated network 830, can encompass any desired configuration, angle, or other characteristic (such as but not limited to a serpentine section). Indeed, fluid reservoirs 850A and 850B can be provided along microscale channels 831, 832, 833, and 834, respectively, if desired. As shown in FIG. 8, fluid reservoirs 850A and 850B comprise at least one dimension that is greater than a dimension of the channels that are immediately adjacent to them.

Continuing, then, with reference to FIG. 8, microscale channels 832 and 834 intersect at intersecting point 860A and proceed into a single microscale channel 835. Microscale channel 835 proceeds to a chamber 870, which in the embodiment shown in FIG. 8, is dimensioned to be wider than microscale channel 835. In some embodiments, chamber 870 comprises a reaction chamber. In some embodiments, chamber 870 comprises a mixing region. In some embodiments, chamber 870 comprises a separation region. In some embodiments, the separation region comprises a given dimension, e.g., length, of a channel, wherein the material is separated by charge, or mass, or combinations thereof, or any other physical characteristic wherein a separation can occur over a given dimension. In some embodiments, the separation region comprises an active material 880. As would be understood by one of ordinary skill in the art, the term “active material” is used herein for convenience and does not imply that the material must be activated to be used for its intended purpose. In some embodiments, the active material comprises a chromatographic material. In some embodiments, the active material comprises a target material.

Continuing with FIG. 8, it is noted that chamber 870 does not necessarily need to be of a wider dimension than an adjacent microscale channel. Indeed chamber 870 can simply comprise a given segment of a microscale channel wherein at least two materials are separated, mixed, and/or reacted. Extending from chamber 870 substantially opposite from microscale channel 835 is microscale channel 836. Microscale channel 836 forms a T-junction with microscale channel 837, which extends away from and is in fluid communication with aperture 810C. Thus, the junction of microscale channels 836 and 837 form intersecting point 860B. Microscale channel 838 extends from intersecting point 860B in a direction substantially opposite microscale channel 837 and to fluid reservoir 850C. Fluid reservoir 850C is dimensioned to be wider than microscale channel 838 for a predetermined length. As noted above, however, a given section of a microscale channel can act as a fluid reservoir without the need to necessarily change a dimension of the section of microscale channel. Moreover, microscale channel 838 could act as a reaction chamber in that a reagent flowing from microscale channel 837 to intersection point 860B could react with a reagent moving from microscale channel 836 to intersection point 860B and into microscale channel 838.

Continuing with reference to FIG. 8, microscale channel 839 extends from fluid reservoir 850C substantially opposite microfluidic channel 838 and travels through a bend into microscale channel 840. Microscale channel 840 is fluidly connected to outlet aperture 810D. Outlet aperture 810D can optionally be reversibly sealed via seal 820D, as discussed above. Again, the reversible sealing of outlet aperture 810D can be desirable in the case of an embodiment where a reaction product is formed in microfluidic device 800 and is desired to be transported to another location in microfluidic device 800.

The flow of a material can be directed through the integrated network 830 of microscale channels, including channels, fluid reservoirs, and reaction chambers through the use of pressure-actuated valves and the like known in the art, for example those described in U.S. Pat. No. 6,408,878 to Unger et al., which is incorporated herein by reference in its entirety. The presently disclosed subject matter thus provides a method of flowing a material through a PFPE-based microfluidic device. In some embodiments, the method comprises providing a microfluidic device comprising (i) a perfluoropolyether (PFPE) material having a characteristic selected from the group consisting of: a viscosity greater than about 100 centistokes (cSt); a viscosity less than about 100 cSt, provided that the liquid PFPE precursor material having a viscosity less than 100 cSt is not a free-radically photocurable PFPE material; (ii) a functionalized PFPE material; (iii) a fluoroolefin-based elastomer; and (iv) combinations thereof, and wherein the microfluidic device comprises one or more microscale channels; and flowing a material in the microscale channel.

Also provided is a method of mixing two or more materials. In some embodiments, the method comprises providing a microscale device comprising (i) a perfluoropolyether (PFPE) material having a characteristic selected from the group consisting of: a viscosity greater than about 100 centistokes (cSt); a viscosity less than about 100 cSt, provided that the liquid PFPE precursor material having a viscosity less than 100 cSt is not a free-radically photocurable PFPE material; (ii) a functionalized PFPE material; (iii) a fluoroolefin-based elastomer; and (iv) combinations thereof; and contacting a first material and a second material in the device to mix the first and second materials. Optionally, the microscale device is selected from the group consisting of a microfluidics device and a microtiter plate.

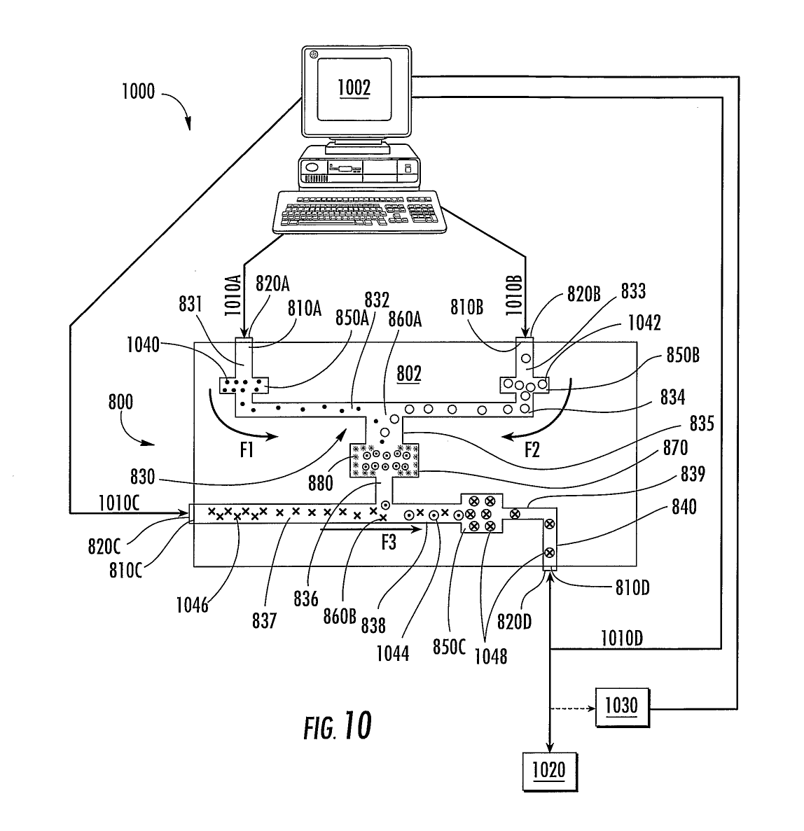

In some embodiments, the method comprises disposing a material in the microfluidic device. In some embodiments, as is best shown in FIG. 10 and as discussed in more detail herein below, the method comprises applying a driving force to move the material along the microscale channel.

In some embodiments, the layer of PFPE material covers a surface of at least one of the one or more microscale channels. Optionally, the layer of PFPE material comprises a functionalized surface. In some embodiments, the microfluidic device comprises one or more patterned layers of PFPE material, and wherein the one or more patterned layers of the PFPE material defines the one or more microscale channels. In this case the patterned layer of PFPE can comprise a functionalized surface. In some embodiments, the microfluidic device can further comprise a patterned layer of a second polymeric material, wherein the patterned layer of the second polymeric material is in operative communication with the at least one of the one or more patterned layers of PFPE material. See FIG. 2.

In some embodiments, the method comprises at least one valve. In some embodiments the valve is a pressure-actuated valve, wherein the pressure-actuated valve is defined by one of: (a) a microscale channel; and (b) at least one of the plurality of holes. In some embodiments, the pressure-actuated valve is actuated by introducing a pressurized fluid into one of: (a) a microscale channel; and (b) at least one of the plurality of holes.

In some embodiments, the pressurized fluid has a pressure between about 10 psi and about 40 psi. In some embodiments, the pressure is about 25 psi. In some embodiments, the material comprises a fluid. In some embodiments, the fluid comprises a solvent. In some embodiments, the solvent comprises an organic solvent. In some embodiments, the material flows in a predetermined direction along the microscale channel.

In the case of mixing two materials, which in some embodiments can comprise mixing two reactants to provide a chemical reaction, the contacting of the first material and the second material is performed in a mixing region defined in the one or more microscale channels. The mixing region can comprise a geometry selected from the group consisting of a T-junction, a serpentine, an elongated channel, a microscale chamber, and a constriction. Optionally, the first material and the second material are disposed in separate channels of the microfluidic device. Also, the contacting of the first material and the second material can be performed in a mixing region defined by an intersection of the channels.

Continuing with a method of mixing, the method can comprise flowing the first material and the second material in a predetermined direction in the microfluidic device, and can comprise flowing the mixed materials in a predetermined direction in the microfluidic device. In some embodiments, the mixed material can be contacted with a third material to form a second mixed material. In some embodiments the mixed material comprises a reaction product and the reaction product can be subsequently reacted with a third reagent. One of ordinary skill in the art upon review of the presently disclosed subject matter would recognize that the description of the method of mixing provided immediately hereinabove is for the purposes of illustration and not limitation. Accordingly, the presently disclosed method of mixing materials can be used to mix a plurality of materials and form a plurality of mixed materials and/or a plurality of reaction products. The mixed materials, including but not limited to reaction products, can be flowed to an outlet aperture of the microfluidic device. A driving force can be applied to move the materials through the microfluidic device. See FIG. 10. In some embodiments the mixed materials are recovered.

In an embodiment employing a microtiter plate, the microtiter plate can comprise one or more wells. In some embodiments, the layer of PFPE material covers a surface of at least one of the one or more wells. The layer of PFPE material can comprise a functionalized surface. See FIG. 5B.

X.B. Method of Synthesizing a Biopolymer in a PFPE-Based Microfluidic Device

In some embodiments, the presently disclosed PFPE-based microfluidic device can be used in biopolymer synthesis, for example, in synthesizing oligonucleotides, proteins, peptides, DNA, and the like. In some embodiments, such biopolymer synthesis systems comprise an integrated system comprising an array of reservoirs, fluidic logic for selecting flow from a particular reservoir, an array of channels, reservoirs, and reaction chambers in which synthesis is performed, and fluidic logic for determining into which channels the selected reagent flows.

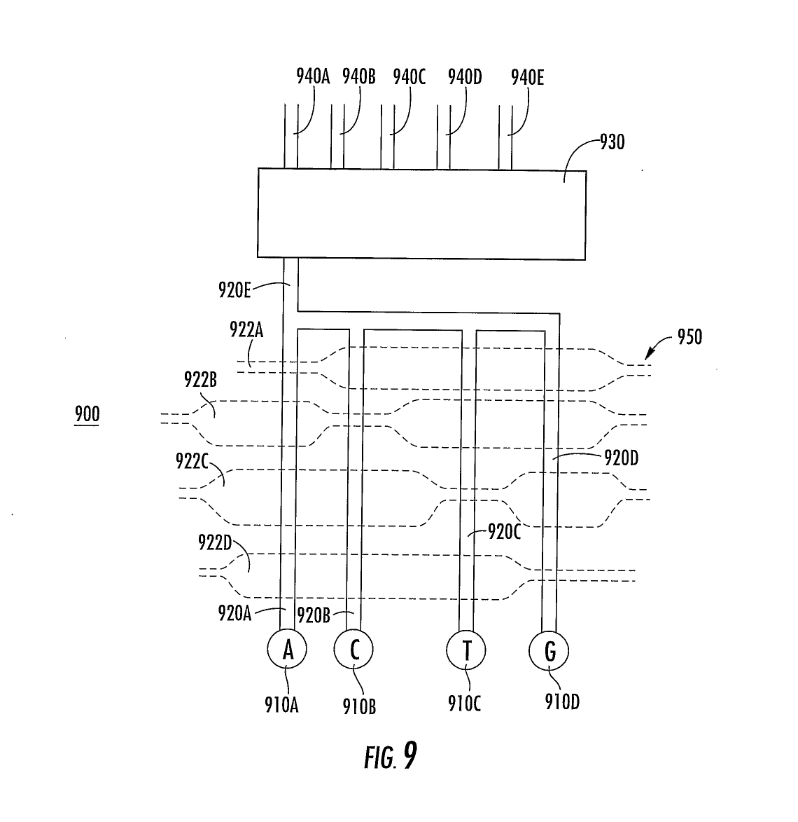

Referring now to FIG. 9, a plurality of reservoirs, e.g., reservoirs 910A, 910B, 910C, and 910D, have bases A, C, T, and G respectively disposed therein, as shown. Four flow channels 920A, 920B, 920C, and 920D are connected to reservoirs 910A, 910B, 910C, and 910D. Four control channels 922A, 922B, 922C, and 922D (shown in phantom) are disposed thereacross with control channel 922A permitting flow only through flow channel 920A (i.e., sealing flow channels 920B, 920C, and 920D), when control channel 922A is pressurized. Similarly, control channel 922B permits flow only through flow channel 920B when pressurized. As such, the selective pressurization of control channels 922A, 922B, 922C, and 922D sequentially selects a desired base A, C, T, and G from a desired reservoir 910A, 910B, 910C, or 910D. The fluid then passes through flow channel 920E into a multiplexed channel flow controller 930, (including, for example, any system as shown in FIG. 8) which in turn directs fluid flow into one or more of a plurality of synthesis channels or reaction chambers 940A, 940B, 940C, 940D, or 940E in which solid phase synthesis can be carried out.

In some embodiments, instead of starting from the desired base A, C, T, and G, a reagent selected from one of a nucleotide and a polynucleotide is disposed in at least one of reservoir 910A, 910B, 910C, and 910D. In some embodiments, the reaction product comprises a polynucleotide. In some embodiments, the polynucleotide is DNA.

Accordingly, after a review of the present disclosure, one of ordinary skill in the art would recognize that the presently disclosed PFPE-based microfluidic device can be used to synthesize biopolymers, as described in U.S. Pat. Nos. 6,408,878 to Unger et al. and 6,729,352 to O'Conner et al., and/or in a combinatorial synthesis system as described in U.S. Pat. No. 6,508,988 to van Dam et al., each of which is incorporated herein by reference in its entirety.

X.C. Method of Incorporating a PFPE-Based Microfluidic Device into an Integrated Fluid Flow System.

In some embodiments, the method of performing a chemical reaction or flowing a material within a PFPE-based microfluidic device comprises incorporating the microfluidic device into an integrated fluid flow system. Referring now to FIG. 10, a system for carrying out a method of flowing a material in a microfluidic device and/or a method of performing a chemical reaction in accordance with the presently disclosed subject matter is schematically depicted. The system itself is generally referred to at 1000. System 1000 can comprise a central processing unit 1002, one or more driving force actuators 1010A, 1010B, 1010C, and 1010D, a collector 1020, and a detector 1030. In some embodiments, detector 1030 is in fluid communication with the microfluidic device (shown in shadow). System microfluidic device 1000 of FIG. 8, and these reference numerals of FIG. 8 are employed in FIG. 10. Central processing unit (CPU) 1002 can be, for example, a general purpose personal computer with a related monitor, keyboard or other desired user interface. Driving force actuators 1010A, 1010B, 1010C, and 1010D can be any suitable driving force actuator as would be apparent to one of ordinary skill in the art upon review of the presently disclosed subject matter. For example, driving force actuators 1010A, 1010B, 1010C, and 1010D can be pumps, electrodes, injectors, syringes, or other such devices that can be used to force a material through a microfluidic device. Representative driving forces themselves thus include capillary action, pump driven fluid flow, electrophoresis based fluid flow, pH gradient driven fluid flow, or other gradient driven fluid flow.

In the schematic of FIG. 10 driving force actuator 1010D is shown as connected at outlet aperture 810D, as will be described below, to demonstrate that at least a portion of the driving force can be provided at the end point of the desired flow of solution, reagent, and the like. Collector 1020 also is provided to show that a reaction product 1048, as discussed below, can be collected at the end point of system flow. In some embodiments, collector 1020 comprises a fluid reservoir. In some embodiments, collector 1020 comprises a substrate. In some embodiments, collector 1020 comprises a detector. In some embodiments, collector 1020 comprises a subject in need of therapeutic treatment. For convenience, system flow is generally represented in FIG. 10 by directional arrows F1, F2, and F3.

Continuing with reference to FIG. 10, in some embodiments a chemical reaction is performed in integrated flow system 1000. In some embodiments, material 1040, e.g, a chemical reagent, is introduced to microfluidic device 1000 through aperture 810A, while a second material 1042, e.g., a second chemical reagent, is introduced to microfluidic device 1000, via inlet aperture 810B. Optionally, microfluidics device 1000 comprises a functionalized surface (see FIG. 5A). Driving force actuators 1010A and 1010B propel chemical reagents 1040 and 1042 to microfluidic channels 831 and 833, respectively. Flow of chemical reagents 1040 and 1042 continues to fluid reservoirs 850A and 850B, where a reserve of reagents 1040 and 1042 is collected. Flow of chemical reagents 1040 and 1042 continues into microfluidic channels 832 and 834 to intersection point 860A wherein initial contact between chemical reagents 1040 and 1042 occurs. Flow of chemical reagents 1040 and 1042 then continues to reaction chamber 870 where a chemical reaction between chemical reagents 1040 and 1042 proceeds.

Continuing with reference to FIG. 10, reaction product 1044 flows to microscale channel 836 and to intersection point 860B. Chemical reagent 1046 then reacts with reaction product 1044 beginning at intersection point 860B through reaction chamber 838 and to fluid reservoir 850C. A second reaction product 1048 is formed. Flow of the second reaction product 1048 continues through microscale channel 840 to aperture 810D and finally into collector 1020. Thus, it is noted that CPU 1002 actuates driving force actuator 1010C such that chemical reagent 1046 is released at an appropriate time to contact reaction product 1044 at intersection point 860B.

X.D. Representative Applications of a Microfluidic Device

In some embodiments, the presently disclosed subject matter discloses a method of screening a sample for a characteristic. In some embodiments, the presently disclosed subject matter discloses a method of dispensing a material. In some embodiments, the presently disclosed subject matter discloses a method of separating a material. Accordingly, one of ordinary skill in the art would recognize that a microfluidic device described herein can be applied to many applications, including, but not limited to, genome mapping, rapid separations, sensors, nanoscale reactions, ink-jet printing, drug delivery, Lab-on-a-Chip, in vitro diagnostics, injection nozzles, biological studies, high-throughput screening technologies, such as for use in drug discovery and materials science, diagnostic and therapeutic tools, research tools, and the biochemical monitoring of food and natural resources, such as soil, water, and/or air samples collected with portable or stationary monitoring equipment.

X.D.1. Method of Screening a Sample for a Characteristic

In some embodiments, the presently disclosed subject matter discloses a method of screening a sample for a characteristic. In some embodiments, the method comprises:
(a) providing a microscale device comprising:
   (i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
   (ii) a functionalized PFPE material;
   (iii) a fluoroolefin-based elastomer; and
   (iv) combinations thereof;
(b) providing a target material;
(c) disposing the sample in the microscale device;
(d) contacting the sample with the target material; and
(e) detecting an interaction between the sample and the target,
   wherein the presence or the absence of the interaction is indicative of the characteristic of the sample.

Referring once again to FIG. 10, at least one of materials 1040 and 1042 comprises a sample. In some embodiments, at least one of materials 1040 and 1042 comprises a target material. Thus, a “sample” generally refers to any material about which information relating to a characteristic is desired. Also, a “target material” can refer to any material that can be used to provide information relating to a characteristic of a sample based on an interaction between the target material and the sample. In some embodiments, for example, when sample 1040 contacts target material 1042 an interaction occurs. In some embodiments, the interaction produces a reaction product 1044. In some embodiments, the interaction comprises a binding event. In some embodiments, the binding event comprises the interaction between, for example, an antibody and an antigen, an enzyme and a substrate, or more particularly, a receptor and a ligand, or a catalyst and one or more chemical reagents. In some embodiments, the reaction product is detected by detector 1030.

In some embodiments, the method comprises disposing the target material in at least one of the plurality of channels. Referring once again to FIG. 10, in some embodiments, the target material comprises active material 880. In some embodiments, the target material, the sample, or both the target and the sample are bound to a functionalized surface. In some embodiments, the target material comprises a substrate, for example a non-patterned layer. In some embodiments, the substrate comprises a semiconductor material. In some embodiments, at least one of the plurality of channels of the microfluidic device is in fluid communication with the substrate, e.g., a non-patterned layer. In some embodiments, the target material is disposed on a substrate, e.g., a non-patterned layer. In some embodiments, at least one of the one or more channels of the microfluidic device is in fluid communication with the target material disposed on the substrate.

In some embodiments, the method comprises disposing a plurality of samples in at least one of the plurality of channels. In some embodiments, the sample is selected from the group consisting of a therapeutic agent, a diagnostic agent, a research reagent, a catalyst, a metal ligand, a non-biological organic material, an inorganic material, a foodstuff, soil, water, and air. In some embodiments, the sample comprises one or more members of one or more libraries of chemical or biological compounds or components. In some embodiments, the sample comprises one or more of a nucleic acid template, a sequencing reagent, a primer, a primer extension product, a restriction enzyme, a PCR reagent, a PCR reaction product, or a combination thereof. In some embodiments, the sample comprises one or more of an antibody, a cell receptor, an antigen, a receptor ligand, an enzyme, a substrate, an immunochemical, an immunoglobulin, a virus, a virus binding component, a protein, a cellular factor, a growth factor, an inhibitor, or a combination thereof.

In some embodiments, the target material comprises one or more of an antigen, an antibody, an enzyme, a restriction enzyme, a dye, a fluorescent dye, a sequencing reagent, a PCR reagent, a primer, a receptor, a ligand, a chemical reagent, or a combination thereof.

In some embodiments, the interaction comprises a binding event. In some embodiments, the detecting of the interaction is performed by at least one or more of a spectrophotometer, a fluorometer, a photodiode, a photomultiplier tube, a microscope, a scintillation counter, a camera, a CCD camera, film, an optical detection system, a temperature sensor, a conductivity meter, a potentiometer, an amperometric meter, a pH meter, or a combination thereof.

Accordingly, after a review of the present disclosure, one of ordinary skill in the art would recognize that the presently disclosed PFPE-based microfluidic device can be used in various screening techniques, such as those described in U.S. Pat. Nos. 6,749,814 to Bergh et al., 6,737,026 to Bergh et al., 6,630,353 to Parce et al., 6,620,625 to Wolk et al., 6,558,944 to Parce et al., 6,547,941 to Kopf-Sill et al., 6,529,835 to Wada et al., 6,495,369 to Kercso et al., and 6,150,180 to Parce et al., each of which is incorporated by reference in its entirety. Further, after a review of the present disclosure, one of ordinary skill in the art would recognize that the presently disclosed PFPE-based microfluidic device can be used, for example, to detect DNA, proteins, or other molecules associated with a particular biochemical system, as described in U.S. Pat. No. 6,767,706 to Quake et al., which is incorporated herein by reference in its entirety.

X.D.2. Method of Dispensing a Material

Additionally, the presently disclosed subject matter describes a method of dispensing a material. In some embodiments, the method comprises:
(a) providing a microfluidic device comprising:
   (i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
   (ii) a functionalized PFPE material;
   (iii) a fluoroolefin-based elastomer; and
   (iv) combinations thereof; and wherein the microfluidics device comprises one or more microscale channels, and wherein at least one of the one or more microscale channels comprises an outlet aperture;
(b) providing at least one material;
(c) disposing at least one material in at least one of the one or more microscale channels; and
(d) dispensing at least one material through the outlet aperture.

In some embodiments, the layer of PFPE material covers a surface of at least one of the one or more microscale channels.

Referring once again to FIG. 10, in some embodiments, a material, e.g., material 1040, second material 1042, chemical reagent 1046, reaction product 1044, and/or reaction product 1048 flow through outlet aperture 810D and are dispensed in or on collector 1020. In some embodiments, the target material, the sample, or both the target and the sample are bound to a functionalized surface.

In some embodiments, the material comprises a drug. In some embodiments, the method comprises metering a predetermined dosage of the drug. In some embodiments, the method comprises dispensing the predetermined dosage of the drug.

In some embodiments, the material comprises an ink composition. In some embodiments, the method comprises dispensing the ink composition on a substrate. In some embodiments, the dispensing of the ink composition on a substrate forms a printed image.

Accordingly, after a review of the present disclosure, one of ordinary skill in the art would recognize that the presently disclosed PFPE-based microfluidic device can be used for microfluidic printing as described in U.S. Pat. Nos. 6,334,676 to Kaszczuk et al., 6,128,022 to DeBoer et al., and 6,091,433 to Wen, each of which is incorporated herein by reference in its entirety.

X.D.3 Method of Separating a Material

In some embodiments, the presently disclosed subject matter describes a method of separating a material, the method comprising:
(a) providing a microfluidic device comprising:
   (i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
   (ii) a functionalized PFPE material;
   (iii) a fluoroolefin-based elastomer; and
   (iv) combinations thereof; and wherein the microfluidics device comprises one or more microscale channels, and wherein at least one of the one or more microscale channels comprises a separation region;
(b) disposing a mixture comprising at least a first material and a second material in the microfluidic device;
(c) flowing the mixture through the separation region; and
(d) separating the first material from the second material in the separation region to form at least one separated material.

Referring once again to FIG. 10, in some embodiments, at least one of material 1040 and second material 1042 comprise a mixture. For example, material 1040, e.g., a mixture, flows through the microfluidic system to chamber 870, which in some embodiments comprises a separation region. In some embodiments, the separation region comprises active material 880, e.g., a chromatographic material. Material 1040, e.g., a mixture, is separated in chamber 870, e.g., a separation chamber, to form a third material 1044, e.g., a separated material. In some embodiments, separated material 1044 is detected by detector 1030.

In some embodiments, the separation region comprises a chromatographic material. In some embodiments, the chromatographic material is selected from the group consisting of a size-separation matrix, an affinity-separation matrix, and a gel-exclusion matrix, or a combination thereof.

In some embodiments, the first or second material comprises one or more members of one or more libraries of chemical or biological compounds or components. In some embodiments, the first or second material comprises one or more of a nucleic acid template, a sequencing reagent, a primer, a primer extension product, a restriction enzyme, a PCR reagent, a PCR reaction product, or a combination thereof. In some embodiments, the first or second material comprises one or more of an antibody, a cell receptor, an antigen, a receptor ligand, an enzyme, a substrate, an immunochemical, an immunoglobulin, a virus, a virus binding component, a protein, a cellular factor, a growth factor, an inhibitor, or a combination thereof.

In some embodiments, the method comprises detecting the separated material. In some embodiments, the detecting of the separated material is performed by at least one or more of a spectrophotometer, a fluorometer, a photodiode, a photomultiplier tube, a microscope, a scintillation counter, a camera, a CCD camera, film, an optical detection system, a temperature sensor, a conductivity meter, a potentiometer, an amperometric meter, a pH meter, or a combination thereof.

Accordingly, after a review of the present disclosure, one of ordinary skill in the art would recognize that the presently disclosed PFPE-based microfluidic device can be used to separate materials, as described in U.S. Pat. Nos. 6,752,922 to Huang et al., 6,274,089 to Chow et al., and 6,444,461 to Knapp et al., each of which is incorporated herein by reference in its entirety.

XI. Applications for Functionalized Microfluidic Devices

Fluidic microchip technologies are increasingly being used as replacements for traditional chemical and biological laboratory functions. Microchips that perform complex chemical reactions, separations, and detection on a single device have been fabricated. These “lab-on-a-chip” applications facilitate fluid and analyte transport with the advantages of reduced time and chemical consumption and ease of automation.

A variety of biochemical analysis, reactions, and separations have been performed within microchannel systems. High throughput screening assays of synthesized molecules and natural products are of great interest. Microfluidic devices for screening a wide variety of molecules based on their ability to inhibit the interactions of enzymes and fluorescently labeled substrates have been described (U.S. Pat. No. 6,046,056, to Parse et al.). As described by Parse et al., such devices allow for screening natural or synthetic libraries of potential drugs through their antagonist or agonist properties. The types of molecules that can be screened include, but are not limited to, small organic or inorganic molecules, polysaccharides, peptides, proteins, nucleic acids or extracts of biological materials such as bacteria, fungi, yeast, plants and animal cells. The analyte compounds can be free in solution or attached to a solid support, such as agarose, cellulose, dextran, polystyrene, carboxymethyl cellulose, polyethylene glycol (PEG), filter paper, nitrocellulose, ion exchange resins, plastic films, glass beads, polyaminemethylvinylether maleic acid copolymer, amino acid copolymer, ethylene-maleic acid copolymer, nylon, silk, and the like. Compounds can be tested as pure compounds or in pools. For example, U.S. Pat. No. 6,007,690 to Nelson et al. relates to a microfluidic molecular diagnostic that purifies DNA from whole blood samples. The device uses an enrichment channel that cleans up or concentrates the analyte sample. For example, the enrichment channel could hold antibody coated beads to remove various cell parts via their antigenic components or could hold chromatographic components, such as ion exchange resin or a hydrophobic or hydrophilic membrane. The device also can comprise a reactor chamber, wherein various reactions can be performed on the analyte, such as a labeling reaction or in the case of a protein analyte, a digestion reaction. Further, U.S. Published Patent Application No. 20040256570 to Beebe et al. describes a device where antibody interaction with an antigenic analyte material coated on the outside of a liposome is detected when that interaction causes the lysis of the liposome and its release of a detectable molecule. U.S. Published Patent Application No. 20040132166 to Miller et al. provides a microfluidic device that can sense environmental factors, such as pH, humidity, and O₂ levels critical for the growth of cells. The reaction chambers in these devices can function as bioreactors capable of growing cells, allowing for their use to transfect cells with DNA and produce proteins, or to test for the possible bioavailability of drug substances by measuring their absorbance across CACO-2 cell layers.

In addition of growing cells, microfluidic devices also have been used to sort cells. U.S. Pat. No. 6,592,821 to Wada et al. describes hydrodynamic focusing to sort cells and subcellular components, including individual molecules, such as nucleic acids, polypeptides or other organic molecules, or larger cell components like organelles. The method can sort for cell viability or other cellular expression functions.

Amplification, separation, sequencing, and identification of nucleic acids and proteins are common microfluidic device applications. For example, U.S. Pat. No. 5,939,291 to Loewy et al. illustrate a microfluidic device that uses electrostatic techniques to perform isothermal nucleic acid amplification. The device can be used in conjunction with a number of common amplification reaction strategies, including PCR (polymerase chain reaction), LCR (ligase chain reaction), SDA (strand displacement amplification), NASBA (nucleic acid sequence-based amplification), and TMA (transcription-mediated amplification). U.S. Pat. No. 5,993,611 to Moroney et al. describes a device that uses capacitive charging to analyze, amplify or otherwise manipulate nucleic acids. Devices have been designed that sort DNA by size, analyzing restriction fragment length polymorphism (see U.S. Pat. No. 6,833,242 to Quake et al.). The devices also can have particular use in forensic applications, such as DNA fingerprinting. U.S. Pat. No. 6,447,724 to Jensen et al. describes microfluidics that identify components of a mixture based on the different fluorescent lifetimes of the labels attached to members of the mixture. Such a device could be used to analyze sequencing reactions of nucleic acids, proteins or oligosaccharides or to inspect or interrogate members of a combinatorial library of organic molecules.

Other microfluidic devices directed toward specific protein applications include a device that promotes protein crystal growth in microfluidic channels (see U.S. Pat. No. 6,409,832, to Weigl et al.). In the device, protein sample and solvent are directed to a channel with laminar flow characteristics that form diffusion zones, which provide well-defined crystallization. U.S. Published Patent Application No. 2004/0121449 to Pugia et al. illustrates a device that can separate red blood cells from plasma using minimal centrifugal force on sample sizes as small as 5 microliters. The device could be particularly useful in clinical diagnostics and also could be used to separate any particulate matter from a liquid.

As partly described hereinabove, microfluidic devices have been utilized as microreactors for a variety of chemical and biological applications. Chambers in these devices can be used for sequencing, restriction enzyme digests, restriction fragment length polymorphism (RFLP) analysis, nucleic acid amplification, or gel electrophoresis (see U.S. Pat. No. 6,130,098, to Handique et al.). A multitude of chemical titration reactions can be run in the devices (see U.S. Published Patent Application No. 20040258571, to Lee et al.), including acid-based titrations or titrations based on precipitation (for example, Ag(I) with Cl⁻, Br⁻, I⁻, or SCN⁻), complex formation (for example, Ag(I) with CN⁻), or redox reactions (such as Fe(II)/Fe(III) with Ce(III)/Ce(IV)). Further, a sensor for potentiometry, amperometry, spectrophotometry, turbidometry, fluorimetry or calorimetry can be attached to the device. Fractionation of proteins (see U.S. Published Patent Application No. 20040245102, to Gilbert et al.) based physical or biological properties is of use in protein expression analysis (finding molecular markers, determining a molecular basis or profile for a disease state or interpreting protein structure/function relationships). A variety of electrophoresis techniques (including capillary isoelectric focusing, capillary zone electrophoresis, and capillary gel electrophoresis) have been employed in microfluidic devices for fractionating proteins (see U.S. Pat. No. 6,818,112, to Schneider et al.). The different electrophoretic techniques can be used in series, with or without a labeling step to help with quantitation, and in conjunction with a variety of elution techniques (such as hydrodynamic salt mobilization, pH mobilization, or electroosmotic flow) to further separate proteins. A variety of other materials have been used to aid in separation processes in microfluidic devices. Such materials may be attached to channel walls in a device or be present as a separate matrix inside a channel (see U.S. Pat. No. 6,581,441 to Paul; U.S. Pat. No. 6,613,581, to Wada et al.). Parallel separation channels can exist to separate many samples at the same time. The solid separation media can be present as a discrete particle or as a porous monolithic solid. Possible materials include silica gel, agarose-based gels, polyacrylamide gels, a colloidal solution, such as a gelatin, starches, non-ionic macroreticular and macroporous resins (such as AMBERCHROM™ (Rohm and Haas Co, Philadelphia, Pa., United States of America), AMBERLITE™ (Rohm and Haas Co, Philadelphia, Pa., United States of America), DOWEX™ (The Dow Chemical Company, Midland, Mich., United States of America), DUOLITE® (Rohm and Haas Co, Philadelphia, Pa., United States of America), and the like), or material present as beads (glass, metal, silica, acrylic, SEPHAROSE™, cellulose, ceramic, polymer, and the like). These materials also can have present on their surfaces various biologically based molecules to aid in separation (for example, lectins bind to carbohydrates and antibodies can bind to antigenic groups on different proteins). Membranes within microchannels have been used for electroosmotic separation (see U.S. Pat. No. 6,406,605, to Moles). Suitable membranes can be comprised of materials, such as track etched polycarbonate or polyimide.

Temperature, concentration and flow gradients also have been employed to aid in separation in microfluidic devices. U.S. Published Patent Application No. 20040142411 to Kirk et al. discloses the use of chemotaxis (the movement of cells induced by a concentration gradient of a soluble chemotactic stimulus), hapatotaxis (the movement of cells in response to a concentration gradient of a substrate-bound stimulus) and chemoinvasion (the movement of cells into and/or through a barrier or gel matrix in response to a stimulus). Chemotatic stimuli include chemorepellants and chemoattractants. A chemoattractant is any substance that attracts cells. Examples include, but are not limited to, hormones such as epinephrine and vasopressin; immunological agents such as interleukein-2; growth factors, chemokines, cytokines, and various peptides, small molecules and cells. Chemorepellants include irritants such as benzalkonium chloride, propylene glycol, methanol, acetone, sodium dodecyl sulfate, hydrogen peroxide, 1-butanol, ethanol and dimethylsulfoxide; toxins, such as cyanide, carbonylcyanide chlorophenylhydrozone; endotoxins and bacterial lipopolysaccharides; viruses; pathogens; and pyrogens. Non-limiting examples of cells that can be manipulated by these techniques include lymphocytes, monocytes, leukocytes, macrophages, mast cells, T-cells, B-cells, neutrophils, basophils, fibroblasts, tumor cells and many others.

Microfluidic devices as sensors have garnered attention in the last few years. Such microfluidic sensors can include dye-based detection systems, affinity-based detections systems, microfabricated gravimetric analyzers, CCD cameras, optical detectors, optical microscopy systems, electrical systems, thermocouples, thermoresistors, and pressure sensors. Such devices have been used to detect biomolecules (see Published PCT International Application No. WO 2004/094,986 to Althaus et al.), including polynucleotides, proteins and viruses through their interaction with probe molecules capable of providing an electrochemical signal. For example, intercalation of a nucleic acid sample with a probe molecule, such as doxorubicin can reduce the amount of free doxorubicin in contact with an electrode; and a change in electrical signal results. Devices have been described that contain sensors for detecting and controlling environmental factors inside device reaction chambers such as humidity, pH, dissolved O₂ and dissolved CO₂ (see Published PCT International Application No. WO 2004/069,983 to Rodgers et al.). Such devices have particular use in growing and maintaining cells. The carbon content of samples can be measured in a device (see U.S. Pat. No. 6,444,474 to Thomas et al.) wherein UV irradiation oxidizes organics to CO₂, which is then quantitated by conductivity measurements or infrared methods. Capacitance sensors used in microfluidic devices (see Published PCT International Application No. WO 2004/085,063 to Xie et al.) can be used to measure pressure, flow, fluid levels, and ion concentrations.

Another application for microfluidic systems includes the high throughput injection of cells (see Published PCT International Application No. WO 00/20554 to Garman et al.) In such a device, cells are impelled to a needle where they can be injected with a wide variety of materials including molecules and macromolecules, genes, chromosomes, or organelles. The device also can be used to extract material from cells and would be of use in a variety of fields, such as gene therapy, pharmaceutical or agrochemical research, and diagnostics. Microfluidic devices also have been used as a means of delivering ink in ink-jet printing (see U.S. Pat. No. 6,575,562 to Anderson et al.), and to direct sample solutions onto an electrospray ionization tip for mass spectrometry (see U.S. Pat. No. 6,803,568 to Bousse et al.). Systems for transdermal drug delivery also have been reported (see Published PCT International Application No. WO 2002/094,368 to Cormier et al.), as well as devices containing light altering elements for use in spectroscopy applications (see U.S. Pat. No. 6,498,353 to Nagle et al.).

X11. Applications for Functionalized Microtiter Plates

The presently disclosed materials and methods also can be applied to the design and manufacture of devices to be used in the manner of microtiter plates. Microtiter plates have a variety of uses in the fields of high throughput screening for proteomics, genomics and drug discovery, environmental chemistry assays, parallel synthesis, cell culture, molecular biology and immunoassays. Common base materials used for microtiter plates include hydrophobic materials, such as polystyrene and polypropylene, and hydrophilic materials, such as glass. Silicon, metal, polyester, polyolefin and polytetrafluoroethylene surfaces also have been used for microtiter plates.

Surfaces can be selected for a particular application based on their solvent and temperature compatibilities and for their ability (or lack of ability) to interact with the molecules or biomolecules being assayed or otherwise manipulated. Chemical modification of the base material is often useful in tailoring the microtiter plate to its desired function either by modifying the surface characteristics or by providing a site for the covalent attachment of a molecule or biomolecule. The functionalizable nature of the presently disclosed materials is well suited for these purposes.

Some applications call for surfaces with low binding characteristics. Proteins and many other biomolecules (such as eukaryotic and microbial cells) can passively adsorb to polystyrene through hydrophobic or ionic interactions. Some surface-modified base materials have been developed to address this problem. Corning® Ultra Low Attachment (Corning Incorporated—Life Sciences, Acton, Massachusetts, United States of America) is a hydrogel-coated polystyrene. The hydrogel coating renders the surface neutral and hydrophilic, preventing the attachment of almost all cells. Vessels made from the surface have uses in preventing serum protein absorption, in preventing anchorage-dependent cells (MDCK, VERO, C6, and the like) from dividing, in selectively culturing tumor or virally transformed cells as unattached colonies, in preventing stem cells from attachment-mediated differentiation, and in studying the activation and inactivation mechanisms of macrophages. NUNC MINISORP™ (Nalgene Nunc International, Naperville, Ill., United States of America) is polyethylene-based product with low protein affinity and has uses for DNA probe and serum-based assays where non-specific binding is a problem.

For other applications base, materials have been modified to enhance their ability to adhere to cells and other biomolecules. NUNCLON Δ™ (Nalgene Nunc International) is a polystyrene surface treated by corona or plasma discharge to add surface carboxyl groups, rendering the material hydrophilic and negatively charged. The material has been used in the cell culture of a variety of cells. Polyolefin and polyester materials also have been treated to enhance their hydrophilicity and thereby become good surfaces for the adhesion and growth of cells (for example PERMANOX™ and THERMANOX™, also from Nalgene Nunc International). Base materials can be coated with poly-D-lysine, collagen or fibronectin to create a positively charged surface, which also can enhance cell attachment, growth and differentiation.

Further, other molecules can be absorbed to a microtiter-like plate. Nunc MAXISORP™ (Nalgene Nunc) is a modified polystyrene base that has a high affinity for polar molecules and is recommended for surfaces where antibodies need to be absorbed to the surface, as in the case of many ELISA assays. Surfaces also can be modified to interact with analytes in a more specific manner. Examples of such functional modifications include nickel-chelate modified surfaces for the quantification and detection of histidine-tagged fusion proteins and glutathione-modified surfaces for the capture of GST-tagged fusion proteins. Streptavidin-coated surfaces can be used when working with biotinylated proteins.

Some modified surfaces provide sites for the covalent attachment of various molecules or biomolecules. COVALINK™ NH Secondary Amine surface (Nalgene Nunc International) is a polystyrene surface covered with secondary amines which can bind proteins and peptides through their carboxyl groups via carbodimide chemistry or bind DNA through the formation of a 5′ phosphoramidiate bond (again using carbodimide chemistry). Other molecules, carbohydrates, hormones, small molecules and the like, containing or modified to contain carboxylate groups also can be bound to the surface. Epoxide is another useful moiety for covalently linking groups to surfaces. Epoxide modified surfaces have been used to create DNA chips via the reaction of amino-modified oligonucleotides with surfaces. Surfaces with immobilized oligonucleotides can be of use in high throughput DNA and RNA detection systems and in automated DNA amplification applications.

Other uses for microtiter plates are directed toward modifying the surface to make it more hydrophobic, rendering it more compatible with organic solvents or to reduce the absorption of drugs, usually small organic molecules. For example, Total Drug Analysis assays generally rely on using acetonitrile to precipitate proteins and salts from a plasma or serum sample. The drug being assayed must remain in solution for subsequent quantification. Organic solvent-compatible microtiter plates also have uses as high performance liquid chromatography (HPLC) or liquid chromatography/mass spectrometry/mass spectrometry (LC/MS/MS) prep devices and as combinatorial chemistry or parallel synthesis reaction vessels (either for solution-based or solid phase chemistries). Examples of surfaces for these types of uses include MULTICHEM™ microplates (Whatman, Inc., Florham Park, New Jersey, United States of America) and MULTISCREEN® Solvinert (Millipore, Billerica, Massachusetts, United States of America).

XIII. Method for Using a Functionalized Perfluoropolyether Network as a Gas Separation Membrane

The presently disclosed subject matter provides for the use of a functionalized perfluoropolyether (PFPE) network as a gas separation membrane. In some embodiments, the functionalized PFPE network is used as a gas separation membrane to separate gases selected from the group consisting of CO₂, methane, hydrogen, CO, CFCs, CFC alternatives, organics, nitrogen, methane, H₂S, amines, fluorocarbons, fluoroolefins, and O₂. In some embodiments, the functionalized PFPE network is used to separate gases in a water purification process. In some embodiments, the gas separation membrane comprises a stand-alone film. In some embodiments, the gas separation membrane comprises a composite film.

In some embodiments, the gas separation membrane comprises a co-monomer. In some embodiments, the co-monomer regulates the permeability properties of the gas separation membrane. Further, the mechanical strength and durability of such membranes can be finely tuned by adding composite fillers, such as silica particles and others, to the membrane. Accordingly, in some embodiments, the membrane further comprises a composite filler. In some embodiments, the composite filler comprises silica particles.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### General Considerations

A PFPE microfluidic device has been previously reported by Rolland, J. et al. JACS 2004, 126, 2322-2323, which is incorporated herein by reference in its entirety. The specific PFPE material disclosed in Rolland, J., et al., was not chain extended and therefore did not possess the multiple hydrogen bonds that are present when PFPEs are chain extended with a diisocyanate linker. Nor did the material posses the higher molecular weights between crosslinks that are needed to improve mechanical properties such as modulus and tear strength which are critical to a variety of microfluidics applications. Furthermore, this material was not functionalized to incorporate various moieties, such as a charged species, a biopolymer, or a catalyst.

Herein is described a variety of methods to address these issues. Included in these improvements are methods which describe chain extension, improved adhesion to multiple PFPE layers and to other substrates such as glass, silicon, quartz, and other polymers as well as the ability to incorporate functional monomers capable of changing wetting properties or of attaching catalysts, biomolecules or other species. Also described are improved methods of curing PFPE elastomers which involve thermal free radical cures, two-component curing chemistries, and photocuring using photoacid generators.

### Example 1

A liquid PFPE precursor having the structure shown below (where n=2) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately, a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. Thirdly, a smooth, flat PFPE layer is generated by drawing a doctor's blade across a small drop of the liquid PFPE precursor across a glass slide. The Slide is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on the fully cured PFPE smooth layer on the glass slide and allowed to heat at 120° C. for 15 hours. Small needles can then be placed in the inlets to introduce fluids and to actuate membrane valves as reported by Unger, M. et al. Science. 2000, 288, 113-6.

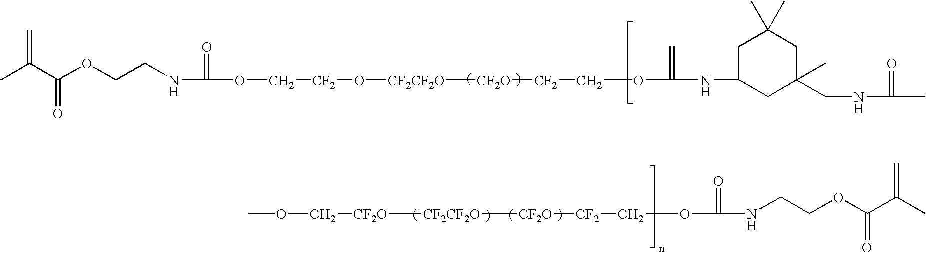

### Example 2

### Thermal Free Radical Glass

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 20 hours under nitrogen purge. The cured layer is then removed, trimmed, and inlet holes are punched through it using a luer stub. The layer is then placed on top of a clean glass slide and fluids are introduced through the inlet holes.

### Example 3

### Thermal Free Radical—Partial Cure Layer to Layer Adhesion

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of about 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 4

### Thermal Free Radical—Partial Cure Adhesion to Polyurethane

A photocurable liquid polyurethane precursor containing methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 5

### Thermal Free Radical—Partial Cure Adhesion to Silicone-Containing Polyurethane

A photocurable liquid polyurethane precursor containing PDMS blocks and methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 6

### Thermal Free Radical—Partial Cure Adhesion to PFPE-PDMS Block Copolymer

A liquid precursor containing both PFPE and PDMS blocks encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 7

### Thermal Free Radical—Partial Cure Glass Adhesion

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 2-3 hours under nitrogen purge. The partially cured layer is removed from the wafer and inlet holes are punched using a luer stub. The layer is then placed on top of a glass slide treated with a silane coupling agent, trimethoxysilyl propyl methacrylate. The layer is then placed in an oven and allowed to heat at 65° C. for 20 hours, permanently bonding the PFPE layer to the glass slide. Small needles can then be placed in the inlets to introduce fluids.

### Example 8

### Thermal Free Radical—Partial Cure PDMS Adhesion

A liquid poly(dimethylsiloxane) precursor poured over a microfluidics master containing 100-μm features in the shape of channels. The wafer is then placed in an oven at 80° C. for 3 hours. Separately, a second master containing 100-μm features in the shape of channels is spin coated with a small drop of liquid PFPE precursor encapped with methacrylate units at 3700 rpm for 1 minute to a thickness of about 20 μm. The wafer is then placed in an oven at 65° C. for 2-3 hours under nitrogen purge. The PDMS layer is then removed, trimmed, and inlet holes are punched through it using a luer stub. The layer is then treated with an oxygen plasma for 20 minutes followed by treatment with a silane coupling agent, trimethoxysilyl propyl methacrylate. The treated PDMS layer is then placed on top of the partially cured PFPE thin layer and heated at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 9

### Thermal Free Radical PDMS Adhesion Using SYLGARD 184® and Functional PDMS

A liquid poly(dimethylsiloxane) precursor is designed such that it can be part of the base or curing component of SYLGARD 184®. The precursor contains latent functionalities such as epoxy, methacrylate, or amines and is mixed with the standard curing agents and poured over a microfluidics master containing 100-μm features in the shape of channels. The wafer is then placed in an oven at 80° C. for 3 hours. Separately, a second master containing 100-μm features in the shape of channels is spin coated with a small drop of liquid PFPE precursor encapped with methacrylate units at 3700 rpm for 1 minute to a thickness of approximately 20 μm. The wafer is then placed in an oven at 65° C. for 2-3 hours under nitrogen purge. The PDMS layer is then removed, trimmed, and inlet holes are punched through it using a luer stub. The PDMS layer is then placed on top of the partially cured PFPE thin layer and heated at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 10

### Epoxy/Amine

A two-component liquid PFPE precursor system such as shown below containing a PFPE diepoxy and a PFPE diamine are blended together in a stoichiometric ratio and poured over a microfluidics master containing 100-μm features in the shape of channels. A PDMS mold is used to contain the liquid in the desired area to a thickness of about 3 mm. The wafer is then placed in an oven at 65° C. for 5 hours. The cured layer is then removed, trimmed, and inlet holes are punched through it using a luer stub. The layer is then placed on top of a clean glass slide and fluids are introduced through the inlet holes.

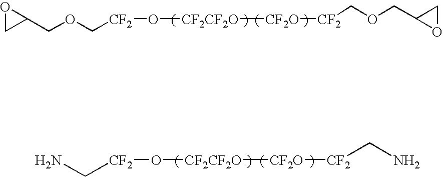

### Example 11

### Epoxy/Amine—Excess Adhesion to Glass

A two-component liquid PFPE precursor system such as shown below containing a PFPE diepoxy and a PFPE diamine are blended together in a 4:1 epoxy:amine ratio such that there is an excess of epoxy and poured over a microfluidics master containing 100-μm features in the shape of channels. A PDMS mold is used to contain the liquid in the desired area to a thickness of about 3 mm. The wafer is then placed in an oven at 65° C. for 5 hours. The cured layer is then removed, trimmed, and inlet holes are punched through it using a luer stub. The layer is then placed on top of a clean glass slide that has been treated with a silane coupling agent, aminopropyltriethoxy silane. The slide is then heated at 65° C. for 5 hours to permanently bond the device to the glass slide. Fluids are then introduced through the inlet holes.

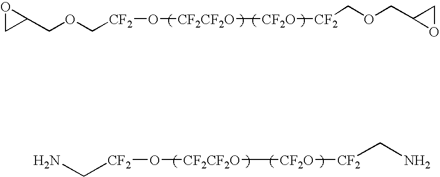

### Example 12

### Epoxy/Amine—Excess Adhesion to PFPE Layers

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger, M. et al. Science. 2000, 288, 113-6.

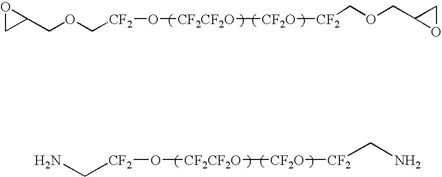

### Example 13

### Epoxy/Amine—Excess Adhesion to PDMS Layers

A liquid poly(dimethylsiloxane) precursor is poured over a microfluidics master containing 100-μm features in the shape of channels. The wafer is then placed in an oven at 80° C. for 3 hours. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The PDMS layer is then removed, trimmed, and inlet holes are punched through it using a luer stub. The layer is then treated with an oxygen plasma for 20 minutes followed by treatment with a silane coupling agent, aminopropyltriethoxy silane. The treated PDMS layer is then placed on top of the PFPE thin layer and heated at 65° C. for 10 hours to adhere the two layers. The thin layer is then trimmed and the adhered layers are lifted from the master. Fluid inlet holes and outlet holes are punched using a luer stub. The bonded layers are then placed on a glass slide treated with aminopropyltriethoxy silane and allowed to heat at 65° C. for 10 hours. Small needles can then be placed in the inlets to introduce fluids and to actuate membrane valves as reported by Unger, M. et al. Science. 2000, 288, 113-6.

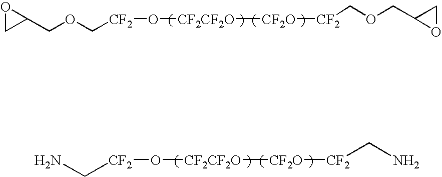

### Example 14

### Epoxy/Amine—Excess Adhesion to PFPE Layers, Attachment of a Biomolecule

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger, M. et al. Science. 2000, 288, 113-6. An aqueous solution containing a protein functionalized with a free amine is then flowed through the channel which is lined with unreacted epoxy moieties, in such a way that the channel is then functionalized with the protein.

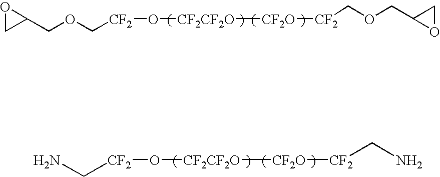

### Example 15

### Epoxy/Amine—Excess Adhesion to PFPE Layers, Attachment of a Charged Species

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger, M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

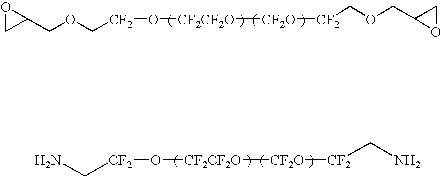

### Example 16

### Epoxy/Amine—Partial Cure Adhesion to Glass

A two-component liquid PFPE precursor system such as shown below containing a PFPE diepoxy and a PFPE diamine are blended together in a stoichiometric ratio and poured over a microfluidics master containing 100-μm features in the shape of channels. A PDMS mold is used to contain the liquid in the desired area to a thickness of about 3 mm. The wafer is then placed in an oven at 65° C. for 0.5 hours such that it is partially cured. The partially cured layer is then removed, trimmed, and inlet holes are punched through it using a luer stub. The layer is then placed on a glass slide treated with a silane coupling agent, aminopropyltriethoxy silane, and allowed to heat at 65° C. for 5 hours such that it is adhered to the glass slide. Small needles can then be placed in the inlets to introduce fluids.

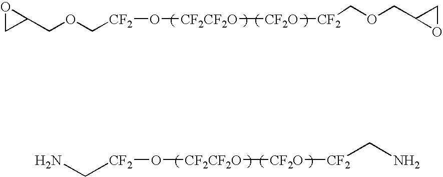

### Example 17

### Epoxy/Amine—Partial Cure Layer to Layer Adhesion

A two-component liquid PFPE precursor system such as shown below containing a PFPE diepoxy and a PFPE diamine are blended together in a stoichiometric ratio and poured over a microfluidics master containing 100-μm features in the shape of channels. A PDMS mold is used to contain the liquid in the desired area to a thickness of about 3 mm. The wafer is then placed in an oven at 65° C. for 0.5 hours such that it is partially cured. The partially cured layer is then removed, trimmed, and inlet holes are punched through it using a luer stub. Separately, a second master containing 100-μm features in the shape of channels is spin coated with a small drop of the liquid PFPE precursors over top of it at 3700 rpm for 1 minute to a thickness of about 20 μm. The wafer is then placed in an oven at 65° C. for 0.5 hours such that it is partially cured. The thick layer is then placed on top of the 20-μm thick layer and aligned in the desired area to form a seal. The layers are then placed in an oven and allowed to heat at 65° C. for 1 hour to adhere the two layers. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 10 hours. Small needles can then be placed in the inlets to introduce fluids and to actuate membrane valves as reported by Unger, M. et al. Science. 2000, 288, 113-6.

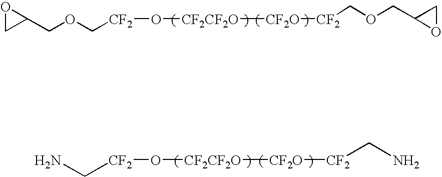

### Example 18

### Epoxy/Amine—Partial Cure PDMS Adhesion

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately, a second master containing 100-μm features in the shape of channels is spin coated with a small drop of liquid PFPE precursors mixed in a stoichiometric ratio at 3700 rpm for 1 minute to a thickness of about 20 μm. The wafer is then placed in an oven at 65° C. for 0.5 hours. The treated PDMS layer is then placed on top of the partially cured PFPE thin layer and heated at 65° C. for 1 hour. The thin layer is then trimmed and the adhered layers are lifted from the master. Fluid inlet holes and outlet holes are punched using a luer stub. The bonded layers are then placed on a glass slide treated with aminopropyltriethoxy silane and allowed to heat at 65° C. for 10 hours. Small needles can then be placed in the inlets to introduce fluids and to actuate membrane valves as reported by Unger, M. et al. Science. 2000, 288, 113-6.

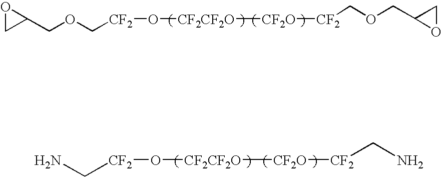

### Example 19

### Photocuring with Latent Functional Groups Available Post Cure Adhesion to Glass

A liquid PFPE precursor having the structure shown below (where R is an epoxy group, the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. The device is placed on a glass slide treated with a silane coupling agent, aminopropyltriethoxy silane, and allowed to heat at 65° C. for 15 hours permanently bonding the device to the glass slide. Small needles can then be placed in the inlets to introduce fluids.

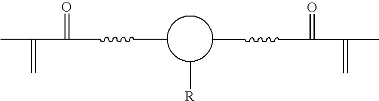

### Example 20

### Photocuring with Latent Functional Groups Available Post Cure Adhesion to PFPE

A liquid PFPE precursor having the structure shown below (where R is an epoxy group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an amine group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6.

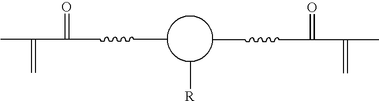

### Example 21

### Photocuring W/Latent Functional Groups Available Post Cure Adhesion to PDMS

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately a second master containing 100-μm features in the shape of channels is spin coated with a small drop of the liquid PFPE precursor (where R is an epoxy) over top of it at 3700 rpm for 1 minute to a thickness of about 20 μm. The wafer is then placed in a UV chamber and exposed to UV light (λ=365) for 10 minutes under a nitrogen purge. The thicker PDMS layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6.

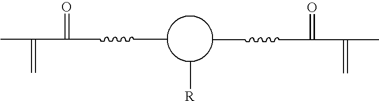

### Example 22

### Photocuring with Latent Functional Groups Available Post Cure Attachment of Biomolecule

A liquid PFPE precursor having the structure shown below (where R is an amine group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a protein functionalized with a free amine is then flowed through the channel which is lined with unreacted epoxy moieties, in such a way that the channel is then functionalized with the protein.

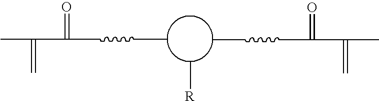

### Example 23

### Photocuring with Latent Functional Groups Available Post Cure Attachment of Charged Species

A liquid PFPE precursor having the structure shown below (where R is an amine group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

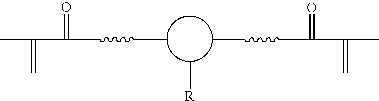

### Example 24

### Photocuring with Functional Monomers Available Post Cure Adhesion to Glass

A liquid PFPE dimethacrylate precursor or a monomethacrylate PFPE macromonomer is blended with a monomer having the structure shown below (where R is an epoxy group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. The device is placed on a glass slide treated with a silane coupling agent, aminopropyltriethoxy silane, and allowed to heat at 65° C. for 15 hours permanently bonding the device to the glass slide. Small needles can then be placed in the inlets to introduce fluids.

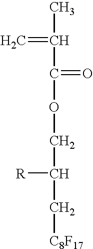

### Example 25

### Photocuring with Functional Monomers Available Post Cure Adhesion to PFPE

A liquid PFPE dimethacrylate precursor is blended with a monomer having the structure shown below (where R is an epoxy group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an amine group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6.

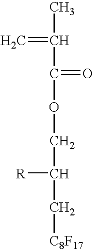

### Example 26

### Photocuring with Functional Monomers Available Post Cure Adhesion to PDMS

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately a second master containing 100-μm features in the shape of channels is spin coated with a small drop of a liquid PFPE dimethacrylate precursor plus functional monomer (where R is an epoxy) plus a photoinitiator over top of it at 3700 rpm for 1 minute to a thickness of about 20 μm. The wafer is then placed in a UV chamber and exposed to UV light (λ=365) for 10 minutes under a nitrogen purge. The thicker PDMS layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6.

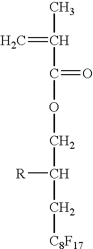

### Example 27

### Photocuring with Functional Monomers Available Post Cure Attachment of a Biomolecule

A liquid PFPE dimethacrylate precursor is blended with a monomer having the structure shown below (where R is an amine group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a protein functionalized with a free amine is then flowed through the channel which is lined with unreacted epoxy moieties, in such a way that the channel is then functionalized with the protein.

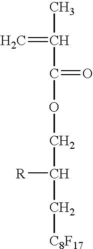

### Example 28

### Photocuring with Latent Functional Groups Available Post Cure Attachment of Charged Species

A liquid PFPE dimethacrylate precursor is blended with a monomer having the structure shown below (where R is an amine group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (A=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

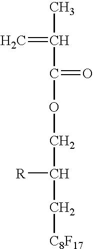

### Example 29

### Fabrication of a PFPE Microfluidic Device Using Sacrificial Channels

A smooth, flat PFPE layer is generated by drawing a doctor's blade across a small drop of the liquid PFPE dimethacrylate precursor across a glass slide. The Slide is then placed in a UV chamber and exposed to UV light (λ=365) for 10 minutes under a nitrogen purge. A scaffold composed of poly(lactic acid) in the shape of channels is laid on top of the flat, smooth layer of PFPE. A liquid PFPE dimethacrylate precursor is with 1 wt % of a free radical photoinitiator and poured over the scaffold. A PDMS mold is used to contain the liquid in the desired area to a thickness of about 3 mm. The apparatus is then placed in a UV chamber and exposed to UV light (λ=365) for 10 minutes under a nitrogen purge. The device is then heated at 150° C. for 24 hours to degrade the poly(lactic acid) thus revealing voids left in the shape of channels.

### Example 30

### Adhesion of a PFPE Device to Glass Using 185-nm Light

A liquid PFPE dimethacrylate precursor is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on a clean, glass slide in such a way that it forms as seal. The apparatus is exposed to 185 nm UV light for 20 minutes, forming a permanent bond between the device and the glass. Small needles can then be placed in the inlets to introduce fluids and to actuate membrane valves as reported by Unger, M. et al. Science. 2000, 288, 113-6.

### Example 31

### “Epoxy Casing” Method to Encapsulate Devices

A liquid PFPE dimethacrylate precursor is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on a clean, glass slide in such a way that it forms as seal. Small needles can then be placed in the inlets to introduce fluids and to actuate membrane valves as reported by Unger, M. et al. Science. 2000, 288, 113-6. The entire apparatus is then encased in a liquid epoxy precursor which is poured over the device allowed to cure. The casing serves to mechanically bind the device the substrate.

### Example 32

### Fabrication of a PFPE Device from a Three-Armed PFPE Precursor

A liquid PFPE precursor having the structure shown below (where the circle represents a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. Thirdly a smooth, flat PFPE layer is generated by drawing a doctor's blade across a small drop of the liquid PFPE precursor across a glass slide. The Slide is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on the fully cured PFPE smooth layer on the glass slide and allowed to heat at 120° C. for 15 hours. Small needles can then be placed in the inlets to introduce fluids and to actuate membrane valves as reported by Unger, M. et al. Science. 2000, 288, 113-6.

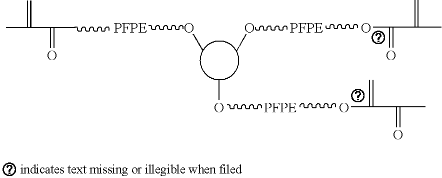

### Example 33

### Photocured PFPE/PDMS Hybrid

A master containing 100-μm features in the shape of channels is spin coated with a small drop of the liquid PFPE dimethacrylate precursor containing photoinitiator over top of it at 3700 rpm for 1 minute to a thickness of about 20 μm. A PDMS dimethacrylate containing photoinitiator is then poured over top of the thin PFPE layer to a thickness of 3 mm. The wafer is then placed in a UV chamber and exposed to UV light (λ=365) for 10 minutes under a nitrogen purge. The layer is then removed, trimmed, and inlet holes are punched through it using a luer stub. The hybrid device is then placed on a glass slide and a seal is formed. Small needles can then be placed in the inlets to introduce fluids.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A microfluidic device comprising a perfluoropolyether (PFPE) material, wherein the PFPE material is prepared from a liquid PFPE precursor material having a characteristic selected from the group consisting of (i) a viscosity greater than about 100 centistokes (cSt), (ii) a viscosity less than about 100 cSt, provided that the liquid PFPE precursor material having a viscosity less than 100 cSt is not a free-radically photocurable PFPE material, and (iii) combinations thereof.

2. The microfluidic device of claim 1, wherein the liquid PFPE precursor is end-capped with a polymerizable group.

3. The microfluidic device of claim 2, wherein the polymerizable group is selected from the group consisting of an acrylate, a methacrylate, an epoxy, an amino, a carboxylic, an anhydride, a maleimide, an isocyanato, an olefinic, and a styrenic group.

4. The microfluidic device of claim 1, wherein the liquid PFPE precursor material comprises a backbone structure, wherein the backbone structure is selected from the group consisting of: 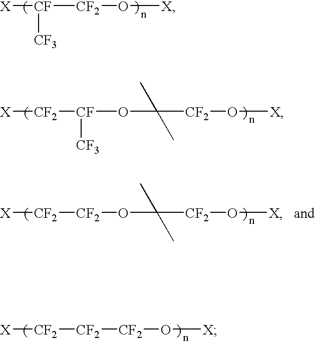
and wherein:
X is present or absent, and when present comprises an endcapping group, and
n is an integer from 1 to 100.

5. The microfluidic device of claim 1, wherein the liquid PFPE precursor material comprises the following structure: 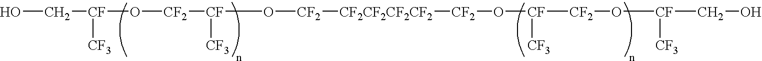
wherein n is an integer from 1 to 100.

6. The microfluidic device of claim 1, wherein the liquid PFPE precursor material comprises the following structure: 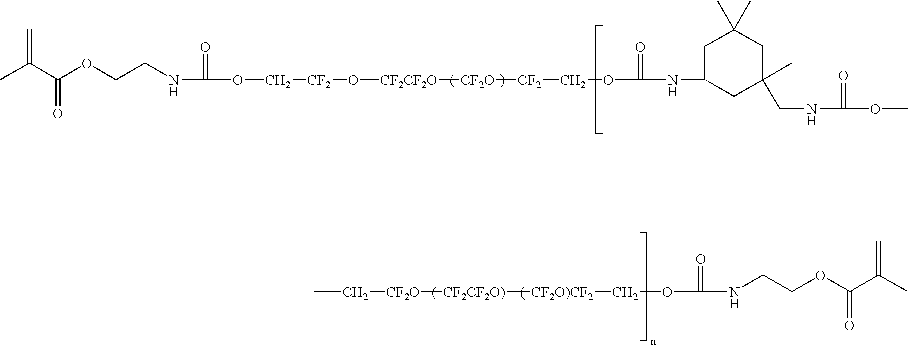
wherein n is an integer from 1 to 100.

7. The microfluidic device of claim 1, wherein the liquid PFPE precursor material comprises a compound comprising the following structure: 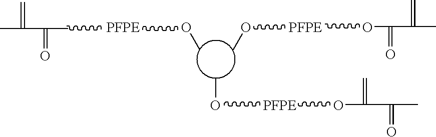
wherein:
the circle comprises a multifunctional linking molecule; and
PFPE comprises a perfluoropolyether chain.

8. The microfluidic device of claim 1, wherein the liquid PFPE precursor material comprises a hyperbranched PFPE liquid precursor material.

9. The microfluidic device of claim 1, wherein the liquid PFPE material comprises an end-functionalized material selected from the group consisting of:

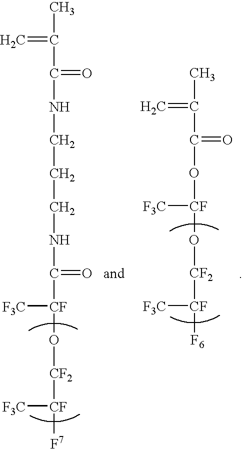

10. The microfluidic device of claim 1, wherein the liquid PFPE material comprises a functional monomer.

11. The microfluidic device of claim 10, wherein the functional monomer is selected from the group consisting of a styrene, a methacrylate, an acrylate, acrylamide, acrylonitrile, and vinyl pyridine.

12. The microfluidic device of claim 11, wherein the styrene is selected from the group consisting of pentafluorostyrene, bromostyrene, chlorostyrene, styrene sulfonic acid, fluorostyrene, and styrene acetate.

13. The microfluidic device of claim 11, wherein the methacrylate is selected from the group consisting of tert-butyl methacrylate, dimethylaminopropyl methacrylate, glycidyl methacrylate, hydroxy ethyl methacrylate, aminopropyl methacrylate, a cyano methacrylate, a trimethoxysilane methacrylate, isocyanato methacrylate, a lactone-containing methacrylate, a sugar-containing methacrylate, polyethylene glycol methacrylate, a nornornane-containing methacrylate, polyhedral oligomeric silsesquioxane methacrylate, 2-trimethylsiloxyethyl methacrylate, and 1H,1H,2H,2H-fluoroctylmethacrylate.

14. The microfluidic device of claim 11, wherein the acrylate is selected from the group consisting of tert-butyl acrylate, allyl acrylate, a cyano acrylate, a trimethoxysilane acrylate, a lactone-containing acrylate, a sugar-containing acrylate, poly-ethylene glycol methacrylate, and a nornornane-containing acrylate.

15. The microfluidic device of claim 1, wherein the liquid PFPE precursor material comprises a two-component liquid PFPE precursor system comprising a mixture of two functionalized PFPE components blended in a stoichiometric ratio.

16. The microfluidic device of claim 15, wherein the two-component PFPE precursor system comprises a mixture of components selected from the group consisting of: an epoxy/amine mixture, a hydroxyl/isocyanate mixture, a hydroxyl/acid chloride mixture, and a hydroxyl/chlorosilane mixture.

17. The microfluidic device of claim 16, wherein epoxy/amine mixture comprises a PFPE diepoxy compound comprising the following structure: 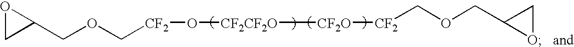
a PFPE diamine compound comprising the following structure:

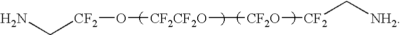

18. The microfluidic device of claim 16, wherein the epoxy/amine mixture comprises a stoichiometric ratio ranging from about 4:1 epoxy:amine to about 1:4 epoxy:amine.

19. The microfluidic device of claim 1, wherein the liquid PFPE precursor material is blended with a functional species, wherein the functional species is mechanically entangled into a PFPE network upon curing.

20. The microfluidic device of claim 1, wherein the perfluoropolyether (PFPE) material comprises a thermally-cured liquid PFPE precursor material.

21. The microfluidic device of claim 1, wherein the perfluoropolyether (PFPE) material comprises a chemically-cured liquid PFPE precursor material.

22. The microfluidic device of claim 1, wherein the perfluoropolyether (PFPE) material comprises a photoacid-cured liquid PFPE precursor material.

23. The microfluidic device of claim 1, wherein the PFPE material is transparent to one of UV light, visible light, and combinations thereof.

24. A microfluidic device comprising a fluoroolefin-based elastomer, wherein the fluoroolefin-based elastomer comprises a first monomer and at least one additional monomer, wherein the first monomer and the at least one additional monomer are different, and wherein:
(a) the first monomer is selected from the group consisting of vinylidene fluoride and tetrafluoroethylene; and
(b) the at least one additional monomer is selected from the group consisting of a fluorine-containing olefin, a fluorine containing vinyl ether, a hydrocarbon olefin; and combinations thereof.

25. The microfluidic device of claim 24, wherein the fluorine-containing olefin is selected from the group consisting of vinylidine fluoride, hexafluoropropylene (HFP), tetrafluoroethylene (TFE), 1,2,3,3,3-pentafluoropropene (1-HPFP), chlorotrifluoroethylene (CTFE), and vinyl fluoride.

26. The microfluidic device of claim 24, wherein the fluorine-containing vinyl ether comprises a perfluoro(alkyl vinyl)ether.

27. The microfluidic device of claim 24, wherein the hydrocarbon olefin is selected from the group consisting of ethylene and propylene.

28. The microfluidic device of claim 24, wherein the fluoroolefin-based elastomer comprises copolymerized units of:
vinylidene fluoride and hexafluoropropylene;
vinylidene fluoride, hexafluoropropylene and tetrafluoroethylene;
vinylidene fluoride, hexafluoropropylene, tetrafluoroethylene and 4-bromo-3,3,4,4-tetrafluorobutene-1;
vinylidene fluoride, hexafluoropropylene, tetrafluoroethylene and 4-iodo-3,3,4,4-tetrafluorobutene-1;
vinylidene fluoride, perfluoro(methyl vinyl)ether, tetrafluoroethylene and 4-bromo-3,3,4,4-tetrafluorobutene-1;
vinylidene fluoride, perfluoro(methyl vinyl)ether, tetrafluoroethylene and 4-iodo-3,3,4,4-tetrafluorobutene-1;
vinylidene fluoride, perfluoro(methyl vinyl)ether, tetrafluoroethylene and 1,1,3,3,3-pentafluoropropene;
tetrafluoroethylene, perfluoro(methyl vinyl)ether and ethylene;
tetrafluoroethylene, perfluoro(methyl vinyl)ether, ethylene and 4-bromo-3,3,4,4-tetrafluorobutene-1;
tetrafluoroethylene, perfluoro(methyl vinyl)ether, ethylene and 4-iodo-3,3,4,4-tetrafluorobutene-1;
tetrafluoroethylene, propylene and vinylidene fluoride;
tetrafluoroethylene and perfluoro(methyl vinyl)ether;
tetrafluoroethylene, perfluoro(methyl vinyl)ether and perfluoro(8-cyano-5-methyl-3,6-dioxa-1-octene);
tetrafluoroethylene, perfluoro(methyl vinyl)ether and 4-bromo-3,3,4,4-tetrafluorobutene-1;
tetrafluoroethylene, perfluoro(methyl vinyl)ether and 4-iodo-3,3,4,4-tetrafluorobutene-1; and
tetrafluoroethylene, perfluoro(methyl vinyl)ether and perfluoro(2 phenoxypropyl vinyl)ether.

29. The microfluidic device of claim 24, wherein the fluoroolefin-based elastomer comprises at least one cure site monomer.

30. The method of claim 29, wherein the cure site monomer is selected from the group consisting of a bromine-containing olefin, an iodine-containing olefin, a bromine-containing vinyl ether, an iodine-containing vinyl ether, a fluorine-containing olefin comprising a nitrile group, a fluorine-containing vinyl ether comprising a nitrile group, 1,1,3,3,3-pentafluoropropene (2-HPFP), perfluoro(2-phenoxypropyl vinyl)ether, and a non-conjugated diene.

31. The microfluidic device of claim 24, wherein the fluoroolefin-based elastomer is transparent to one of UV light, visible light, and combinations thereof.

32. The microfluidic device of claim 24 wherein the fluoroolefin-based elastomer has a Mooney viscosity less than about 40 (ML 1+10 at 121° C.).

33. The microfluidic device of claim 24, wherein the fluoroolefin-based elastomer is permeable to oxygen, carbon dioxide, and nitrogen.

34. A method for functionalizing the surface of a microscale device, the method comprising forming a layer of a functionalized material, wherein the functionalized material is selected from the group consisting of a liquid PFPE precursor material and a liquid fluoroolefin-based precursor material.

35. The method of claim 34, wherein the layer of functionalized material comprises a latent functional group that is not reacted during a curing process.

36. The method of claim 35, wherein the latent functional group comprises a methacrylate group.

37. The method of claim 34, wherein the layer of functionalized material comprises a latent functional group that is introduced in the generation of the liquid precursor material.

38. The method of claim 37, wherein the latent functional group comprises a methacrylate group.

39. The method of claim 34, wherein the layer of functionalized material comprises a two-component liquid PFPE precursor material, wherein the two-component liquid PFPE precursor material comprises a mixture of two functionalized PFPE components blended in a stoichiometric ratio.

40. The method of claim 34, wherein the layer of functionalized material comprises a chemical linker group.

41. The method of claim 40, wherein the chemical linker group comprises the following structure: 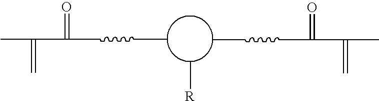
wherein:
R comprises an epoxy group;
the circle comprises a linking molecule; and
the wavy line comprises a PFPE chain.

42. The method of claim 34, wherein the layer of functionalized material comprises a functional monomer.

43. The method of claim 42, wherein the functional monomer is selected from the group consisting of tert-butyl methacrylate, tert butyl acrylate, dimethylaminopropyl methacrylate, glycidyl methacrylate, hydroxy ethyl methacrylate, aminopropyl methacrylate, allyl acrylate, a cyano acrylate, a cyano methacrylate, a trimethoxysilane acrylate, a trimethoxysilane methacrylate, isocyanato methacrylate, a lactone-containing acrylate, a lactone-containing methacrylate, a sugar-containing acrylate, a sugar-containing methacrylate, polyethylene glycol methacrylate, a nornornane-containing methacrylate, a nornornane-containing acrylate, polyhedral oligomeric silsesquioxane methacrylate, 2-trimethylsiloxyethyl methacrylate, 1H,1H,2H,2H-fluoroctylmethacrylate, pentafluorostyrene, vinyl pyridine, bromostyrene, chlorostyrene, styrene sulfonic acid, fluorostyrene, styrene acetate, acrylamide, and acrylonitrile.

44. The method of claim 34, wherein the layer of functionalized material is functionalized by exposure to a plasma.

45. The method of claim 44, wherein the plasma is selected from the group consisting of an Argon plasma and an oxygen plasma.

46. The method of claim 34, wherein the layer of functionalized material is functionalized by exposure to UV radiation.

47. The method of claim 34, comprising attaching a functional moiety to the layer of functionalized material.

48. The method of claim 47, wherein the functional moiety is selected from the group consisting of a protein, an oligonucleotide, a drug, a catalyst, a dye, a sensor, an analyte, and a charged species capable of changing the wettability of the channel.

49. The method of claim 34, wherein the layer of functionalized material comprises a microfluidic channel.

50. The method of claim 34, comprising adhering the layer of functionalized material to a substrate.

51. The method of claim 50, wherein the substrate comprises a microtiter well.

52. A layer of functionalized material prepared by the method of claim 34.

53. A method for forming a multilayer device, the method comprising:
(a) providing a first layer of material, wherein the first layer of material comprises a material selected from the group consisting of a liquid perfluoropolyether (PFPE) precursor, a poly(dimethylsiloxane) (PDMS) precursor, a polyurethane precursor, a polyurethane precursor comprising PDMS blocks, a precursor comprising PFPE and PDMS blocks, and a fluoroolefin-based precursor; and
(b) contacting the first layer of material with:
(i) a substrate;
(ii) a second layer of material, wherein the second layer of material comprises a material selected from the group consisting of a perfluoropolyether (PFPE) precursor, a poly(dimethylsiloxane) (PDMS) precursor, a polyurethane precursor, a polyurethane precursor comprising PDMS blocks, a precursor comprising PFPE and PDMS blocks, and fluoroolefin-based precursor; and wherein the second layer of material can be the same as or different than the first layer of material; and
(iii) combinations thereof;
to form a multilayer device.

54. The method of claim 53, wherein the first layer of material comprises a fully-cured material.

55. The method of claim 53, wherein the contacting of the first layer of material with the substrate forms a reversible seal.

56. The method of claim 53, wherein the first layer of material comprises a partially-cured material.

57. The method of claim 56, wherein the partially-cured material comprises a partially-cured PFPE precursor material encapped with a methacrylate group.

58. The method of claim 53, comprising treating the substrate with a silane coupling agent to form a treated substrate.

59. The method of claim 58, wherein the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

60. The method of claim 56, comprising:
(a) contacting of the first layer of partially-cured material with the treated substrate; and
(b) treating the first layer of partially cured material to form a bond between the first layer of partially-cured material and the treated substrate.

61. The method of claim 53, wherein:
(a) the first layer of material comprises a first partially-cured material; and
(b) the second layer of material comprises a second partially-cured material, wherein the first partially-cured material and the second partially-cured material can be the same or different.

62. The method of claim 61, comprising:
(a) contacting the first layer of partially-cured material with the second layer of partially-cured material to form a partially-cured multilayer device; and
(b) treating the partially-cured multilayer device to form a fully-cured multilayer device.

63. The method of claim 62, wherein the treating comprises a process selected from the group consisting of a thermal curing process, a chemical curing process, a photoacid curing process, and a catalytic curing process.

64. The method of claim 62, wherein the first layer of partially-cured material and second layer of partially-cured material each comprise a thermally-curable PFPE precursor material.

65. The method of claim 62, wherein the first layer of partially-cured material comprises a polyurethane precursor material and the second layer of partially-cured material comprises a PFPE precursor material.

66. The method of claim 62, wherein the first layer of partially-cured material comprises a polyurethane precursor comprising poly(dimethylsiloxane) blocks and the second layer of partially-cured material comprises a PFPE precursor material.

67. The method of claim 62, wherein the first layer of partially-cured material comprises a precursor material comprising a PFPE block and a PDMS block and the second layer of partially-cured material comprises a PFPE precursor material.

68. The method of claim 62, wherein the first layer of partially-cured material comprises a PDMS precursor and the second layer of partially-cured material comprises a PFPE precursor material.

69. The method of claim 68, wherein the PFPE precursor material is encapped with a methacrylate group.

70. The method of claim 68, comprising treating the PDMS precursor with a plasma treatment followed by treatment with a silane coupling agent.

71. The method of claim 70, wherein the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

72. The method of claim 62, comprising:
(a) contacting the partially-cured multilayer structure with a substrate, wherein the substrate is coated with a partially-cured precursor material to form a second partially-cured multilayer device; and
(b) treating the second partially-cured multilayer device to form a second fully-cured multilayer device.

73. The method of claim 72, wherein the treating comprises a process selected from the group consisting of a thermal curing process, a chemical curing process, a photoacid curing process, and a catalytic curing process.

74. The method of claim 53, wherein at least one of the first layer of material and the second layer of material comprises a material formed from a two-component PFPE precursor material, wherein the two-component PFPE precursor material comprises a mixture of two functionalized PFPE components blended in a stoichiometric ratio.

75. The method of claim 74, wherein the two-component PFPE precursor system comprises a mixture of components selected from the group consisting of an epoxy/amine mixture, a hydroxyl/isocyanate mixture, a hydroxyl/acid chloride mixture, and a hydroxyl/chlorosilane mixture.

76. The method of claim 75, wherein epoxy/amine mixture comprises a PFPE diepoxy compound comprising the following structure: 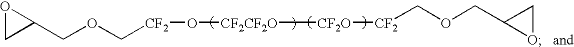
a PFPE diamine compound comprising the following structure:

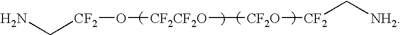

77. The method of claim 75, wherein the epoxy/amine mixture comprises a stoichiometric ratio ranging from about 4:1 epoxy:amine to about 1:4 epoxy:amine.

78. The method of claim 77, wherein the stoichiometric ratio is about 4:1 epoxy:amine.

79. The method of claim 78, comprising:
(a) providing a substrate, wherein the substrate is treated with a silane coupling agent;
(b) contacting the first layer of material formed from a two-component PFPE precursor material comprising a stoichiometric ratio of about 4:1 epoxy:amine with the substrate; and
(b) treating first layer of material and the substrate to form a multilayer device.

80. The method of claim 79, wherein the silane coupling agent comprises aminopropyltriethoxy silane.

81. The method of claim 77, wherein the stoichiometric ratio is about 1:4 epoxy:amine.

82. The method of claim 81, comprising:
(i) providing a first layer of material comprising a stoichiometric ratio of about 1:4 epoxy:amine;
(ii) contacting the first layer of material comprising a stoichiometric ratio of about 1:4 epoxy:amine with a second layer of material comprising a stoichiometric ratio of about 4:1 epoxy:amine; and
(iii) treating the two layers of material to form a multilayer device.

83. The method of claim 78, comprising:
(i) providing a first layer of PDMS material;
(ii) treating the first layer of PDMS material with plasma treatment followed by treatment with a silane coupling agent to form a treated layer of PDMS material;
(iii) contacting the treated layer of PDMS material with a second layer of material comprising a stoichiometric ratio of about 4:1 epoxy:amine; and
(iv) treating the two layers of material to form a multilayer device.

84. The method of claim 83, wherein the silane coupling agent comprises aminopropyltriethoxy silane.

85. The method of claim 74, comprising:
(a) providing a first layer of material formed from a two-component PFPE precursor material, wherein the two-component PFPE precursor material comprises a mixture of two functionalized PFPE components blended in a stoichiometric ratio;
(b) treating the first layer of material to form a first layer of partially-cured material;
(c) contacting the first layer of partially-cured material with one of:
(i) a substrate;
(ii) a second layer of material; and
(iii) combinations thereof; and
(d) treating the first layer of partially-cured material to adhere the partially-cured material to one of the substrate, a second layer of material, and combinations thereof.

86. The method of claim 85, wherein the substrate is selected from the group consisting of a glass material, a quartz material, a silicon material, and a fused silica material.

87. The method of claim 86, comprising treating the substrate with a silane coupling agent.

88. The method of claim 87, wherein the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

89. The method of claim 85, wherein the second layer of material comprises a PFPE precursor material.

90. The method of claim 85, wherein the second layer of material comprises a poly(dimethylsiloxane) material, wherein the poly(dimethylsiloxane) material is treated with an oxygen plasma followed by treatment with a silane coupling agent.

91. The method of claim 53, wherein the PFPE precursor material comprises the following structure: 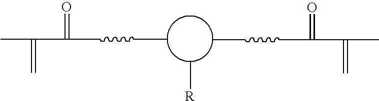
wherein:
R comprises an epoxy group;
the circle comprises a linking molecule; and
the wavy line comprises a PFPE chain.

92. The method of claim 91, comprising photocuring the PFPE precursor material to form a layer of fully-cured PFPE material.

93. The method of claim 92, comprising:
(a) contacting the layer of fully-cured PFPE material with one of:
(i) a substrate;
(ii) a second layer of material; and
(iii) combinations thereof; and
(b) treating the fully-cured material to bond it to one of the substrate, the second layer of material, and combinations thereof.

94. The method of claim 93, wherein the substrate is selected from the group consisting of a glass material, a quartz material, a silicon material, and a fused silica material.

95. The method of claim 94, comprising treating the substrate with a silane coupling agent.

96. The method of claim 95, wherein the silane coupling agent comprises aminopropyltriethoxy silane.

97. The method of claim 93, wherein the second layer of material comprises a PFPE material.

98. The method of claim 93, wherein the second layer of material comprises a treated PDMS material, and wherein the treated PDMS material is treated with an oxygen plasma followed by treatment with a silane coupling agent.

99. The method of claim 98, wherein the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

100. The method of claim 53, comprising blending the PFPE precursor with a functional monomer to form a PFPE precursor blend.

101. The method of claim 100, wherein the functional monomer comprises the following structure:

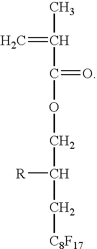

102. The method of claim 100, comprising photocuring the PFPE precursor blend to form a layer of fully-cured PFPE material.

103. The method of claim 102, comprising:
(a) contacting the layer of fully-cured PFPE material with one of:
(i) a substrate;
(ii) a second layer of material; and
(iii) combinations thereof; and
(b) treating the layer of fully-cured material to bond it to one of the substrate, the second layer of material, and combinations thereof.

104. The method of claim 103, wherein the substrate is selected from the group consisting of a glass material, a quartz material, a silicon material, and a fused silica material.

105. The method of claim 104, comprising treating the substrate with a silane coupling agent.

106. The method of claim 105, wherein the silane coupling agent is selected from the group consisting of a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

107. The method of claim 103, wherein the second layer of material comprises a PFPE material.

108. The method of claim 103, wherein the second layer of material comprises a treated PDMS material, and wherein the treated PDMS material is treated with an oxygen plasma followed by treatment with a silane coupling agent.

109. The method of claim 108, wherein the silane coupling agent comprises aminopropyltriethoxy silane.

110. The method of claim 53, wherein the substrate is selected from the group consisting of a glass material, a quartz material, a silicon material, a fused silica material, an elastomeric material, and a rigid thermoplastic material.

111. The method of claim 110, wherein the elastomeric material is selected from the group consisting of poly(dimethylsiloxane) (PDMS), Kratons, buna rubber, natural rubber, a fluorelastomer, chloroprene, butyl rubber, nitrile rubber, polyurethane, or a thermoplastic elastomer.

112. The method of claim 110, wherein the rigid thermoplastic material is selected from the group consisting of polystyrene, poly(methyl methacrylate), a polyester, a polycarbonate, a polyimide, a polyamide, a polyvinylchloride, a polyolefin, a poly(ketone), a poly(ether ether ketone), and a poly(ether sulfone).

113. The method of claim 110, comprising treating the substrate with a silane coupling agent.

114. The method of claim 113, wherein the silane coupling agent is selected from the group consisting of trimethylsilyl propyl methacrylate and aminopropyltriethoxy silane.

115. The method of claim 53, wherein the substrate comprises a microtiter plate.

116. The method of claim 53, wherein the first layer of material comprises at least one microscale channel.

117. The method of claim 53, wherein the first layer of material comprises at least one nanoscale channel.

118. A multilayer device formed by the method of claim 53.

119. The multilayer device of claim 118, wherein the multilayer device comprises a microfluidic device.

120. A method of adhering one of a microscale device, a nanoscale device, and combinations thereof to a substrate, the method comprising:
(a) providing one of a microscale device, a nanoscale device, and combinations thereof, wherein the device comprises a material selected from the group consisting of a perfluoropolyether material and a fluoroolefin-based material;
(b) contacting the device with a substrate;
(c) coating the device and the substrate with a liquid precursor encasing material;
(d) solidifying the liquid precursor encasing material to mechanically bind the device to the substrate.

121. The method of claim 120, wherein the substrate is selected from the group consisting of a glass material, a quartz material, a silicon material, a fused silica material, an elastomeric material, and a rigid thermoplastic material.

122. The method of claim 121, wherein the elastomeric material is selected from the group consisting of poly(dimethylsiloxane) (PDMS), Kratons, buna rubber, natural rubber, a fluorelastomer, chloroprene, butyl rubber, nitrile rubber, polyurethane, or a thermoplastic elastomer.

123. The method of claim 121, wherein the rigid thermoplastic material is selected from the group consisting of polystyrene, poly(methyl methacrylate), a polyester, a polycarbonate, a polyimide, a polyamide, a polyvinylchloride, a polyolefin, a poly(ketone), a poly(ether ether ketone), and a poly(ether sulfone).

124. The method of claim 120, wherein the substrate is treated with a silane coupling agent.

125. The method of claim 124, wherein the silane coupling agent is selected from the group consisting of trimethylsilyl propyl methacrylate and aminopropyltriethoxy silane.

126. The method of claim 120, wherein the solidifying of the liquid precursor encasing material comprises a curing process.

127. The method of claim 120, wherein the liquid precursor encasing material is selected from the group consisting of a liquid epoxy precursor and a polyurethane.

128. A method for forming one of a microstructure, a nanostructure, and combinations thereof, the method comprising:
(a) disposing a first PFPE precursor material on a substrate to form a first layer of liquid PFPE precursor material on the substrate;
(b) treating the first layer of PFPE precursor material to form a first layer of treated PPFE material on the substrate;
(c) placing a multidimensional structure on the first layer of treated PFPE material, wherein the multidimensional structure has a characteristic selected from the group consisting of (i) degradability; (ii) selectively soluble; and (iii) combinations thereof;
(d) encasing the multidimensional structure with a second layer of liquid PFPE precursor material;
(e) treating the second layer of PFPE precursor material to form a second layer of treated PFPE material; and
(f) removing the degradable or selectively soluble material from the second layer of treated PFPE material to form one of a microstructure, a nanostructure, and combinations thereof.

129. The method of claim 128, wherein the degradable or selectively soluble material is selected from the group consisting of a wax, a photoresist, a poly(lactic acid), a polylactone, a polysulfone, a polyelectrolyte, a cellulose fiber, a water soluble polymer, a solvent soluble polymer, a salt, a solid organic compound, and a solid inorganic compound.

130. The method of claim 128, wherein the removing of the degradable or selectively soluble material comprises a process selected from the group consisting of a thermal process, a photochemical process, and a dissolution process.

131. The method of claim 128, comprising mixing at least one of the first PFPE precursor material and the second PFPE precursor material with one of a thermal free radical initiator and a photoinitiator.

132. The method of claim 128, wherein the treating of at least one of the first layer of PFPE precursor material and the second layer of PFPE precursor material comprises a curing process.

133. The method of claim 132, wherein the curing process is selected from the group consisting of a thermal curing process and a photochemical curing process.

134. The method of claim 128, wherein the encasing of the multidimensional structure with a second layer of liquid PFPE precursor material comprises a spin-coating process.

135. A microstructure prepared by the method of claim 128.

136. The microstructure of claim 135, wherein the microstructure comprises a microfluidic channel.

137. A nanostructure prepared by the method of claim 128.

138. The nanostructure of claim 137, wherein the nanostructure comprises a nanoscale channel.

139. A method of forming one of a microstructure, a nanostructure, and combinations thereof, the method comprising:
(a) providing a patterned layer of perfluorinated perfluoropolyether (PFPE) material, wherein the patterned layer of PFPE material comprises a patterned surface;
(b) disposing a predetermined volume of degradable or selectively soluble material on the patterned surface of the patterned layer of PFPE material;
(c) encasing the predetermined volume of degradable or selectively soluble material on the patterned surface of the patterned layer of PFPE material; and
(d) removing the predetermined volume of degradable or selectively soluble material from the patterned surface of the layer of PFPE material to form one of a microscale structure, a nanoscale structure, and combinations thereof.

140. The method of claim 139, wherein the degradable or selectively soluble material is selected from the group consisting of a wax, a photoresist, a poly(lactic acid), a polylactone, a polysulfone, a polyelectrolyte, a cellulose fiber, a water soluble polymer, a solvent soluble polymer, a salt, a solid organic compound, and a solid inorganic compound.

141. The method of claim 140, wherein the removing of the predetermined volume of degradable or selectively soluble material comprises a process selected from the group consisting of a thermal process, a photochemical process, and a dissolution process.

142. A microstructure prepared by the method of claim 139.

143. The microstructure of claim 142, wherein the microstructure comprises a microfluidic channel.

144. A nanostructure prepared by the method of claim 139.

145. The nanostructure of claim 144, wherein the nanostructure comprises a nanoscale channel.

146. A method of flowing a material in a microfluidic device, the method comprising:
(a) providing a microfluidic device comprising at least one layer of
(i) a perfluoropolyether (PFPE) material having a characteristic selected from the group consisting of a viscosity greater than about 100 centistokes (cSt) and a viscosity less than about 100 cSt, provided that the liquid PFPE precursor material having a viscosity less than 100 cSt is not a free-radically photocurable PFPE material;
(ii) a functionalized PFPE material;
(iii) a fluoroolefin-based elastomer; and
(iv) combinations thereof; and
(b) flowing a material in the microscale channel.

147. The method of claim 146, wherein the at least one layer of material covers a surface of at least one of the one or more microscale channels.

148. The method of claim 147, wherein the at least one layer of material comprises a functionalized surface.

149. The method of claim 146, wherein the one or more microscale channels comprises an integrated network of microscale channels.

150. The method of claim 149, wherein the microscale channels of the integrated network intersect predetermined points.

151. The method of claim 146, wherein the microfluidic device comprises one or more patterned layers of a first polymeric material, and wherein the one or more patterned layers of the first polymeric material defines the one or more microscale channels.

152. The method of claim 151, wherein the microfluidic device further comprises a patterned layer of a second polymeric material, wherein the patterned layer of the second polymeric material is in operative communication with the at least one of the one or more patterned layers of the first polymeric material.

153. The method of claim 151, wherein the patterned at least one layer of material comprises a functionalized surface.

154. The method of claim 151, wherein the one or more microscale channels comprises an integrated network of microscale channels.

155. The method of claim 154, wherein the microscale channels of the integrated network intersect predetermined points.

156. The method of claim 151, wherein the patterned layer of the first polymeric material comprises a plurality of holes.

157. The method of claim 156, wherein at least one of the plurality of holes comprises an inlet aperture.

158. The method of claim 156, wherein at least one of the plurality of holes comprises an outlet aperture.

159. The method of claim 156, wherein the microfluidic device comprises one or more valves.

160. The method of claim 146, wherein the material is selected from the group consisting of a fluid, an organic solvent, an aqueous solution, an aqueous solution dispersed in a substantially non-aqueous solvent, a surfactant mixture, and a reaction mixture.

161. The method of claim 146, wherein the material flows in a predetermined direction along the microscale channel.

162. The method of claim 146, comprising applying a driving force to move the material along the microscale channel.

163. A method of mixing two or more materials, the method comprising:
(a) providing a microscale device comprising at least one layer of:
(i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
(ii) a functionalized PFPE material;
(iii) a fluoroolefin-based elastomer; and
(iv) combinations thereof; and
(b) contacting a first material and a second material in the device to mix the first and second materials.

164. The method of claim 163, wherein the microscale device is selected from the group consisting of a microfluidics device and a microtiter plate.

165. The method of claim 164, wherein the microfluidics device comprises one or more microscale channels.

166. The method of claim 165, wherein the at least one layer of material covers a surface of at least one of the one or more microscale channels.

167. The method of claim 166, wherein the at least one layer of material comprises a functionalized surface.

168. The method of claim 165, wherein the microfluidic device comprises at least one patterned layer of a first polymeric material, and wherein the patterned layer of the first polymeric material defines the one or microscale channels.

169. The method of claim 168, wherein the microfluidic device further comprises a patterned layer of a second polymeric material, wherein the patterned layer of the second polymeric material is in operative communication with the at least one of the one or more patterned layers of first polymeric material.

170. The method of claim 168, wherein the patterned layer of first polymeric material comprises a functionalized surface.

171. The method of claim 165, wherein the one or more microscale channels comprises an integrated network of microscale channels.

172. The method of claim 171, wherein the microscale channels of the integrated network intersect at predetermined points.

173. The method of claim 165, wherein the contacting of the first material and the second material is performed in a mixing region defined in the one or more microscale channels.

174. The method of claim 173, wherein the mixing region comprises a geometry selected from the group consisting of a T-junction, a serpentine, an elongated channel, a microscale chamber, and a constriction.

175. The method of claim 165, wherein the first material and the second material are disposed in separate channels of the microfluidic device.

176. The method of claim 175, wherein the contacting of the first material and the second material is performed in a mixing region defined by an intersection of the channels.

177. The method of claim 176, wherein the mixing region comprises a geometry selected from the group consisting of a T-junction, a serpentine, an elongated channel, a microscale chamber, and a constriction.

178. The method of claim 164, comprising flowing the first material and the second material in a predetermined direction in the microfluidic device.

179. The method of claim 164, comprising flowing the mixed materials in a predetermined direction in the microfluidic device.

180. The method of claim 164, comprising contacting the mixed material with a third material to form a second mixed material.

181. The method of claim 164, comprising flowing the mixed materials to an outlet aperture of the microfluidic device.

182. The method of claim 164, comprising applying a driving force to move the materials through the microfluidic device.

183. The method of claim 164, wherein the microtiter plate comprises one or more wells.

184. The method of claim 183, wherein the at least one layer of material covers a surface of at least one of the one or more wells.

185. The method of claim 184, wherein the at least one layer of material comprises a functionalized surface.

186. The method of claim 163, comprising recovering the mixed materials.

187. A method of screening a sample for a characteristic, the method comprising:
(a) providing a microscale device comprising at least one layer of
(i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
(ii) a functionalized PFPE material;
(iii) a fluoroolefin-based elastomer; and
(iv) combinations thereof;
(b) providing a target material;
(c) disposing the sample in the microscale device;
(d) contacting the sample with the target material; and
(e) detecting an interaction between the sample and the target material,
wherein the presence or the absence of the interaction is indicative of the characteristic of the sample.

188. The method of claim 187, wherein the microscale device is selected from the group consisting of a microfluidic device and a microtiter plate.

189. The method of claim 188, wherein the microfluidic device comprises one or more microscale channels.

190. The method of claim 189, wherein the at least one layer of material covers a surface of at least one of the one or more microscale channels.

191. The method of claim 189, wherein the microfluidic device comprises at least one patterned layer of first polymeric material, and wherein the patterned layer of the first polymeric material defines the one or microscale channels.

192. The method of claim 191, wherein the microfluidic device further comprises a patterned layer of a second polymeric material, wherein the patterned layer of the second polymeric material is in operative communication with the at least one of the one or more patterned layers of the first polymeric material.

193. The method of claim 191, wherein the one or more microscale channels comprises an integrated network of microscale channels.

194. The method of claim 193, wherein the microscale channels of the integrated network intersect at predetermined points.

195. The method of claim 188, wherein the microtiter plate comprises one or more wells.

196. The method of claim 195, wherein the at least one layer of material covers a surface of at least one of the one or more wells.

197. The method of claim 187, comprising disposing the target material in the microscale device.

198. The method of claim 197, wherein the target material is bound to the functionalized surface.

199. The method of claim 187, wherein the target material comprises one or more of an antigen, an antibody, an enzyme, a restriction enzyme, a dye, a fluorescent dye, a sequencing reagent, a PCR reagent, a primer, a receptor, a ligand, a chemical reagent, or a combination thereof.

200. The method of claim 187, wherein the sample is bound to the functionalized surface.

201. The method of claim 187, wherein the sample is selected from the group consisting of a therapeutic agent, a diagnostic agent, a research reagent, a catalyst, a metal ligand, a non-biological organic material, an inorganic material, a foodstuff, soil, water, and air.

202. The method of claim 187, wherein the sample comprises one or more members of one or more libraries of chemical or biological compounds or components.

203. The method of claim 187, wherein the sample comprises one or more of a nucleic acid template, a sequencing reagent, a primer, a primer extension product, a restriction enzyme, a PCR reagent, a PCR reaction product, or a combination thereof.

204. The method of claim 187, wherein the sample comprises one or more of an antibody, a cell receptor, an antigen, a receptor ligand, an enzyme, a substrate for an enzyme, an immunochemical, an immunoglobulin, a virus, a virus binding component, a protein, a cellular factor, a growth factor, an inhibitor, or a combination thereof.

205. The method of claim 187, comprising disposing a plurality of samples in the microscale device.

206. The method of claim 187, wherein the interaction comprises a binding event.

207. The method of claim 187, wherein the detecting of the interaction is performed by at least one or more of a spectrophotometer, a fluorometer, a photodiode, a photomultiplier tube, a microscope, a scintillation counter, a camera, a CCD camera, film, an optical detection system, a temperature sensor, a conductivity meter, a potentiometer, an amperometric meter, a pH meter, or a combination thereof.

208. A method of separating a material, the method comprising:
(a) providing a microfluidic device comprising at least one layer of
(i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
(ii) a functionalized PFPE material;
(iii) a fluoroolefin-based elastomer; and
(iv) combinations thereof; and wherein the microfluidics device comprises one or more microscale channels, and wherein at least one of the one or more microscale channels comprises a separation region;
(b) disposing a mixture comprising at least a first material and a second material in the microfluidic device;
(c) flowing the mixture through the separation region; and
(d) separating the first material from the second material in the separation region to form at least one separated material.

209. The method of claim 208, wherein the at least one layer of material covers a surface of at least one of the one or more microscale channels.

210. The method of claim 208, wherein the one or more microscale channels comprises an integrated network of microscale channels.

211. The method of claim 209, wherein the microscale channels of the integrated network intersect predetermined points.

212. The method of claim 208, wherein the microfluidic device comprises one or more patterned layers of a first polymeric material, and wherein the one or more patterned layers of the first polymeric material defines the one or more microscale channels.

213. The method of claim 212, wherein the microfluidic device further comprises a patterned layer of a second polymeric material, wherein the patterned layer of the second polymeric material is in operative communication with the at least one of the one or more patterned layers of the first polymeric material.

214. The method of claim 212, wherein the one or more microscale channels comprises an integrated network of microscale channels.

215. The method of claim 214, wherein the microscale channels of the integrated network intersect predetermined points.

216. The method of claim 208, wherein the separation region comprises a functionalized surface.

217. The method of claim 208, wherein the separation region comprises a chromatographic material.

218. The method of claim 217, wherein the chromatographic material is selected from the group consisting of a size-separation matrix, an affinity-separation matrix; and a gel-exclusion matrix, or a combination thereof.

219. The method of claim 208, wherein the first or second material comprises one or more members of one or more libraries of chemical or biological compounds or components.

220. The method of claim 208, wherein the first or second material comprises one or more of a nucleic acid template, a sequencing reagent, a primer, a primer extension product, a restriction enzyme, a PCR reagent, a PCR reaction product, or a combination thereof.

221. The method of claim 208, wherein the first or second material comprises one or more of an antibody, a cell receptor, an antigen, a receptor ligand, an enzyme, a substrate for an enzyme, an immunochemical, an immunoglobulin, a virus, a virus binding component, a protein, a cellular factor, a growth factor, an inhibitor, or a combination thereof.

222. The method of claim 208, comprising detecting the separated material.

223. The method of claim 222, wherein the detecting of the separated material is performed by at least one or more of a spectrophotometer, a fluorometer, a photodiode, a photomultiplier tube, a microscope, a scintillation counter, a camera, a CCD camera, film, an optical detection system, a temperature sensor, a conductivity meter, a potentiometer, an amperometric meter, a pH meter, or a combination thereof.

224. A method of dispensing a material, the method comprising:
(a) providing a microfluidic device comprising at least one layer of:
(i) a perfluoropolyether (PFPE) material having a characteristic selected from the group consisting of: a viscosity greater than about 100 centistokes (cSt) and a viscosity less than about 100 cSt, provided that the liquid PFPE precursor material having a viscosity less than 100 cSt is not a free-radically photocurable PFPE material;
(ii) a functionalized PFPE material;
(iii) a fluoroolefin-based elastomer; and
(iv) combinations thereof; and wherein the microfluidics device comprises one or more microscale channels, and wherein at least one of the one or more microscale channels comprises an outlet aperture;
(b) providing at least one material;
(c) disposing at least one material in at least one of the one or more microscale channels; and
(d) dispensing at least one material through the outlet aperture.

225. The method of claim 224, wherein the at least one layer of material covers a surface of at least one of the one or more microscale channels.

226. The method of claim 225, wherein the one or more microscale channels comprises an integrated network of microscale channels.

227. The method of claim 226, wherein the microscale channels of the integrated network intersect predetermined points.

228. The method of claim 224, wherein the microfluidic device comprises one or more patterned layers of a first polymeric material, and wherein the one or more patterned layers of the first polymeric material defines the one or more microscale channels.

229. The method of claim 228, wherein the microfluidic device further comprises a patterned layer of a second polymeric material, wherein the patterned layer of the second polymeric material is in operative communication with the at least one of the one or more patterned layers of the first polymeric material.

230. The method of claim 228, wherein the patterned at least one layer of material comprises a functionalized surface.

231. The method of claim 228, wherein the one or more microscale channels comprises an integrated network of microscale channels.

232. The method of claim 231, wherein the microscale channels of the integrated network intersect predetermined points.

233. The method of claim 224, wherein the material comprises a drug.

234. The method of claim 233, comprising metering a predetermined dosage of the drug.

235. The method of claim 234, comprising dispensing the predetermined dosage of the drug.

236. The method of claim 224, wherein the material comprises an ink composition.

237. The method of claim 236, comprising dispensing the ink composition on a substrate.

238. The method of claim 237, wherein the dispensing of the ink composition on a substrate forms a printed image.
